(19) United States
(12) Patent Application Publication
(10) Pub. No.: US 2024/0271119 A1
(43) Pub. Date: Aug. 15, 2024

(54) METHODS OF PERIPLASMIC PHAGE-ASSISTED CONTINUOUS EVOLUTION

(71) Applicants: The Broad Institute, Inc., Cambridge, MA (US); President and Fellows of Harvard College, Cambridge, MA (US)

(72) Inventors: David R. Liu, Cambridge, MA (US); Tina Wang, Cambridge, MA (US); Mary S. Morrison, Cambridge, MA (US)

(73) Assignees: The Broad Institute, Inc., Cambridge, MA (US); President and Fellows of Harvard College, Cambridge, MA (US)

(21) Appl. No.: 18/292,421

(22) PCT Filed: Jul. 27, 2022
(86) PCT No.: PCT/US2022/074208

§ 371 Date: Jan. 26, 2024

Related U.S. Application Data
(60) Provisional application No. 63/226,689, filed on Jul. 28, 2021.

(51) Int. Cl. C12N 15/10; C12M 1/00; C12M 1/34; C12N 1/20; C12N 7/00; C12N 15/70; C12R 1/19

(52) U.S. Cl. CPC C12N 15/1024; C12M 23/58; C12M 29/18; C12M 41/36; C12N 1/205; C12N 7/00; C12N 15/1058; C12N 15/70; C12R 2001/19

(57) ABSTRACT

Aspects of the disclosure relate to compositions, systems, and methods for evolving nucleic acids and proteins utilizing continuous directed evolution in the periplasm of a host cell. In some embodiments, the methods comprise passing a nucleic acid from cell-to-cell in a desired, function dependent manner. The linkage of the desired function and passage of the nucleic acid from cell-to-cell allows for continuous selection and mutation of the nucleic acid.

### RELATED APPLICATIONS

This application claims the benefit under 35 U.S.C § 119(e) of the filing date of U.S. Provisional Application No. 63/226,689, entitled “METHODS OF PERIPLASMIC PHAGE-ASSISTED CONTINUOUS EVOLUTION”, filed Jul. 28, 2021, the entire contents of which are incorporated herein by reference.

### FEDERALLY SPONSORED RESEARCH

This invention was made with government support under Grant Numbers AI142756, EB031172, GM118062, and EB027793, awarded by the National Institutes of Health. The government has certain rights in the invention.

### REFERENCE TO AN ELECTRONIC SEQUENCE LISTING

The contents of the electronic sequence listing (B119570141WO00-SEQ-CBD.xml; Size: 51,735 bytes; and Date of Creation: Jul. 26, 2022) is herein incorporated by reference in its entirety.

### BACKGROUND

Proteins and nucleic acids employ only a small fraction of the available functionality. There is considerable current interest in modifying proteins and nucleic acids to diversify their functionality. Molecular evolution efforts include in vitro diversification of a starting molecule into related variants from which desired molecules are chosen. Methods used to generate diversity in nucleic acid and protein libraries include whole genome mutagenesis (Hart et al., Amer. Chem. Soc. (1999), 121:9887-9888), random cassette mutagenesis (Reidhaar-Olson et al., Meth. Enzymol. (1991), 208:564-86), error-prone PCR (Caldwell, et al. (1992), PCR Methods Applic. (1992), 2: 28-33), DNA shuffling using homologous recombination (Stemmer (1994) Nature (1994), 370:389-391), and phage-assisted continuous evolution (PACE).

### SUMMARY

Phage-assisted continuous evolution (PACE) is a rapid directed evolution system capable of evolving proteins over days or weeks, with minimal human intervention required during evolution process. In PACE, an evolving protein of interest is encoded in place of gene III (gIII) in the genome of a bacteriophage (e.g., M13). An accessory plasmid (AP) within a host E. coli cell expresses gIII under the control of a transcriptional circuit that is activated in response to the desired function of the evolving protein. As phage depend on pIII, the protein product of gIII, to efficiently infect host cells, PACE links the desired property of an evolving protein with the ability of the phage that encodes it to replicate.

Continuous in vivo evolution platforms, including PACE, generally have been limited to evolving proteins in the cytoplasm of the host cell, which is a chemically reducing environment. This limitation inhibits the formation of disulfide linkages between cysteine residues, which linkages are crucial for the stability and proper folding for many proteins, including antibodies and antibody fragments. The loss of a single disulfide bond can dramatically reduce protein stability and abrogate protein function. Loss of stabilizing disulfide bonds often leads to aggregation during cytoplasmic expression, making disulfide-enriched proteins a challenging class of proteins to evolve by currently available continuous directed evolution techniques. While the activity of the target protein may be evolved and observed using this methodology, as the environment does not accurately reflect the conditions the target protein may encounter in clinical or other uses, its measured and observed activity and efficacy also may differ in clinical and other applications.

There have been efforts to address this issue previously. For example, while disulfide bond formation can be supported in the cytosol through expression of a thiol oxidase and a disulfide isomerase in the cytoplasmic space, introducing non-native oxidative chemistry into the bacterial cytoplasm increases cellular stress and can lead to membrane impairment and aggregation. Alternatively, directed evolution can be applied to an evolving protein to compensate for loss of disulfides and render a protein biologically active in the reducing cytoplasm, but this process adds complexity and steps which are not ultimately necessary to proteins intended for use outside the cell. Compensatory stabilizing mutations may also result in trade-off costs to target affinity or other biological functions, limiting the scope and relevance of the resulting proteins for use outside of cells. Finally, binding affinity evolutions in the reducing cytoplasm are limited to interactions in which the target protein being bound does not itself rely on disulfides to fold, excluding disulfide-containing extracellular antigens of therapeutic interest.

Aspects of the disclosure relate to improved methods of continuous evolution which allow for the expression of di-sulfide-containing evolved proteins, and other evolved proteins that require a non-reducing environment to fold and/or function properly. As described further below, the bacterial periplasm, which is an oxidizing environment, supports the formation of disulfides in proteins, such as antibodies and their derivatives. Expression of evolving proteins in the periplasm permits disulfide bond formation while retaining the evolving protein within the bacterial host cell. Linking a protein's desired activity in an oxidizing environment, such as the periplasm to phage propagation enables the continuous evolution of proteins that require a non-reducing environment to function and/or fold properly.

Accordingly, in some aspects, the disclosure provides methods of continuous evolution comprising: (a) contacting a population of bacterial host cells in a culture medium with a population of selection phage comprising a gene of interest to be evolved and lacking a functional pIII gene required for the generation of infectious phage particles; wherein (1) the phage allow for expression of the gene of interest in the host cells; (2) the host cells are suitable host cells for phage infection, replication, and packaging, wherein the phage comprises all phage genes required for the generation of phage particles, except a full-length pIII gene; and (3) the host cells comprise: (i) a first expression construct encoding a fusion protein comprising a DNA binding protein connected to a periplasmic capture agent; and (ii) a second expression construct encoding a pIII protein under the control of a conditional promoter, wherein activation of the conditional promoter is dependent on binding of a first gene product of the gene of interest to the periplasmic capture agent; and (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that are not infected by phage, wherein the binding of the first gene product to the periplasmic capture agent is a desired function, wherein phage expressing gene products having a desired function induce production of pIII and release progeny into the culture medium capable of infecting new host cells, and wherein phage expressing gene products having an undesired function do not produce pIII and release only non-infectious progeny into the culture medium.

In some embodiments, a population of bacterial host cells comprises E. coli cells.

In some embodiments, a population of selection phage comprises filamentous phage. In some embodiments, a population of selection phage comprises M13 phage.

In some embodiments, a gene of interest to be evolved encodes a protein. In some embodiments, the protein to be evolved comprises one or more disulfide bonds. In some embodiments, disulfide bonds are important in the global stability of a protein, for example proteins which have extracellular functions in a tissue of origin, such as receptors and proteases. In some embodiments, the protein is an antibody, antibody fragment, or single-chain variable region (scFv), single-domain antibody, extracellular receptor (e.g., mammalian extracellular receptor), extracellular protease, monobody, adnectin, or nanobody.

In some embodiments, a protein further comprises a capture tag. In some embodiments, a capture tag comprises a peptide. In some embodiments, a capture tag comprises a SH2 domain or a GCN4 leucine zipper domain.

In some embodiments, a DNA binding protein is a bacterial DNA binding protein. In some embodiments, the bacterial DNA binding protein is an E. coli DNA binding protein, such as a CadC protein. In some embodiments, a bacterial DNA binding protein comprises a CadC protein (SEQ ID NO: 33) or a fragment thereof. In some embodiments, a DNA binding protein lacks a periplasmic sensor domain. In some embodiments, a DNA binding protein is encoded by the nucleic acid sequence set forth in SEQ ID NO: 11. In some embodiments, a DNA binding protein comprises the amino acid sequence set forth as

(SEQ ID NO: 42)
MQQPVVRVGEWLVTPSINQISRNGRQLTLEPRLIDLLVFFAQHSGEVLS
RDELIDNVWKRSIVTNHVVTQSISELRKSLKDNDEDSPVYIATVPKRGY
KLMVPVIWYSEEEGEEIMLSSPPPIPEAVPATDSPSHSLNIQNTATPPE
QSPVKSKRGGPGLLLLLLLLLLLLLLLLGPGG.

In some embodiments, a periplasmic capture agent comprises a cognate binding partner of the first gene product. In some embodiments, a periplasmic capture agent comprises an antigen bound by a first gene product. In some embodiments, a periplasmic capture agent comprises an antibody or fragment thereof that binds to a first gene product.

In some embodiments, a periplasmic capture agent comprises a monobody that binds to the first gene product. In some embodiments, a monobody comprises an HA4 monobody.

In some embodiments, a first expression construct further comprises a nucleic acid sequence encoding a portion of a split-intein. In some embodiments, a portion of a split-intein is connected to a portion of a periplasmic signal peptide sequence. In some embodiments, a portion of a periplasmic signal peptide sequence encodes amino acids 1-8 of SEQ ID NO: 32. In some embodiments, a split-intein comprises a Nostoc punctiforme (Npu) trans-splicing DnaE intein N-terminal portion or C-terminal portion. In some embodiments, a split-intein is encoded by the nucleic acid sequence set forth in SEQ ID NO: 19.

In some embodiments, a selection phage further comprises a nucleic acid sequence encoding a portion of a split-intein connected to the gene of interest to be evolved. In some embodiments, a portion of a split-intein is connected to a portion of a periplasmic signal peptide sequence. In some embodiments, a portion of a periplasmic signal peptide sequence encodes amino acids 9-20 of SEQ ID NO: 32. In some embodiments, a split-intein comprises a Nostoc punctiforme (Npu) trans-splicing DnaE intein N-terminal portion or C-terminal portion. In some embodiments, a split-intein is encoded by the nucleic acid sequence set forth in SEQ ID NO: 20.

In some embodiments, a conditional promoter comprises two or more DNA binding protein binding sites. In some embodiments, the two or more binding sites comprise a Cad1 binding site, and a Cad2 binding site. In some embodiments, a conditional promoter comprises a P_{cadBA} promoter. In some embodiments, the conditional promoter comprises the sequence set forth in SEQ ID NO: 10.

In some embodiments, host cells further comprise a mutagenesis plasmid.

In some embodiments, a first expression construct and a second expression construct are situated on the same vector. In some embodiments, a first expression construct and a second expression construct are situated on different vectors. In some embodiments, each vector is a bacterial plasmid.

In some embodiments, methods described herein further comprise isolating the first gene product from the population of host cells.

In some aspects, the disclosure provides a protein evolved by a method as described herein.

In some embodiments, the disclosure provides an isolated nucleic acid comprising a sequence, or encoding a protein having the sequence, as set forth in any one of SEQ ID NO: 1-33.

In some aspects, the disclosure provides an apparatus for continuous evolution of a gene of interest, the apparatus comprising a lagoon comprising a cell culture vessel comprising population of bacterial host cells in a culture medium with a population of selection phage comprising a gene of interest to be evolved and lacking a functional pIll gene required for the generation of infectious phage particles; wherein the phage allow for expression of the gene of interest in the host cells; the host cells are suitable host cells for phage infection, replication, and packaging, wherein the phage comprises all phage genes required for the generation of phage particles, except a full-length pIII gene; and the host cells comprise: a first expression construct encoding a fusion protein comprising a DNA binding protein connected to a periplasmic capture agent; and a second expression construct encoding a pIII protein under the control of a conditional promoter, wherein activation of the conditional promoter is dependent on binding of a first gene product of the gene of interest to the periplasmic capture agent; an inflow connected to a turbidostat; optionally an inflow, connected to a vessel comprising a mutagen; optionally an inflow, connected to a vessel comprising an inducer; an outflow; a controller controlling inflow and outflow rates; a turbidostat comprising a cell culture vessel comprising a population of fresh bacterial host cells; an outflow connected to the inflow of the lagoon; an inflow connected to a vessel comprising liquid media; a turbidity meter measuring the turbidity of the culture of fresh bacterial host cells in the turbidostat; a controller controlling the inflow of sterile liquid media and the outflow into the waste vessel based on the turbidity of the culture liquid; optionally, a vessel comprising mutagen; and optionally, a vessel comprising an inducer.

In some embodiments, phages are M13 phages. In some embodiments, phages do not comprise a full-length pIII gene.

In some embodiments, bacterial host cells are amenable to phage infection, replication, and production.

In some embodiments, bacterial host cells are E. coli cells.

In some embodiments, fresh host cells are not infected by the phage.

In some embodiments, the population of host cells is in suspension culture in liquid media.

In some embodiments, the rate of inflow of fresh host cells and the rate of outflow are substantially the same.

In some embodiments, the rate of inflow and/or the rate of outflow is from about 0.1 lagoon volumes per hour to about 25 lagoon volumes per hour.

In some embodiments, the inflow and outflow rates are controlled based on a quantitative assessment of the population of host cells in the lagoon.

In some embodiments, the quantitative assessment comprises measuring of cell number, cell density, wet biomass weight per volume, turbidity, or growth rate.

In some embodiments, the inflow and/or outflow rate is controlled to maintain a host cell density of from about 10² cells/ml to about 10¹² cells/ml in the lagoon.

In some embodiments, the inflow and/or outflow rate is controlled to maintain a host cell density of about 10² cells/ml, about 10³ cells/ml, about 10⁴ cells/ml, about 10⁵ cells/ml, about 5-10⁵ cells/ml, about 10⁶ cells/ml, about 5.10⁶ cells/ml, about 10⁷ cells/ml, about 5·10⁷ cells/ml, about 10⁸ cells/ml, about 5·10⁸ cells/ml, about 10⁹ cells/ml, about 5·10⁹ cells/ml, about 10¹⁰ cells/ml, about 5·10¹⁰ cells/ml, or more than 10¹⁰ cells/ml, in the lagoon.

In some embodiments, the inflow and outflow rates are controlled to maintain a substantially constant number of host cells in the lagoon.

In some embodiments, the inflow and outflow rates are controlled to maintain a substantially constant frequency of fresh host cells in the lagoon.

In some embodiments, the population of host cells is continuously replenished with fresh host cells that are not infected by the phage.

In some embodiments, the lagoon further comprises an inflow connected to a vessel comprising a mutagen, and wherein the inflow of mutagen is controlled to maintain a concentration of the mutagen in the lagoon that is sufficient to induce mutations in the host cells.

In some embodiments, the mutagen is ionizing radiation, ultraviolet radiation, base analogs, deaminating agents (e.g., nitrous acid), intercalating agents (e.g., cthidium bromide), alkylating agents (e.g., ethylnitrosourea), transposons, bromine, azide salts, psoralen, benzene,3-Chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl)acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide (captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-Aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9) or t-butyl hydroperoxide (BHP) (CAS no. 75-91-2).

In some embodiments, the lagoon comprises an inflow connected to a vessel comprising an inducer. In some embodiments, the inducer induces expression of mutagenesis-promoting genes into host cells.

In some embodiments, the host cells comprise an expression cassette encoding a mutagenesis-promoting gene under the control of an inducible promoter. In some embodiments, the inducible promoter is an arabinose-inducible inducer and wherein the inducer is arabinose.

In some embodiments, the lagoon volume is from approximately 1 ml to approximately 1001.

In some embodiments, the lagoon further comprises a heater and a thermostat controlling the temperature in the lagoon. In some embodiments, the temperature in the lagoon is controlled to be about 37° C.

In some embodiments, the inflow rate and/or the outflow rate are controlled to allow for the incubation and replenishment of the population of host cells for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive phage life cycles. In some embodiments, the time sufficient for one phage life cycle is about 10 minutes.

In some aspects, the disclosure provides a vector system for periplasmic phage-based continuous directed evolution comprising: selection phage comprising a gene of interest to be evolved and lacking a functional pIII gene required for the generation of infectious phage particles; a first expression construct encoding a fusion protein comprising a DNA binding protein connected to a periplasmic capture agent; and, a second expression construct encoding a pIII protein under the control of a conditional promoter, wherein activation of the conditional promoter is dependent on binding of a first gene product of the gene of interest to the periplasmic capture agent.

In some embodiments, the selection phage is an M13 phage. In some embodiments, the selection phage comprises all genes required for the generation of phage particles.

In some embodiments, the phage genome comprises a pI, pII, pIV, pV, pVI, pVII, pVIII, pIX, and a pX gene, but not a full-length pIII gene. In some embodiments, the phage genome comprises an F1 origin of replication. In some embodiments, the phage genome comprises a 3′-fragment of a pIII gene. In some embodiments, the 3′-fragment of the pIII gene comprises a promoter.

In some embodiments, the selection phage comprises a multiple cloning site operably linked to a promoter.

In some embodiments, the gene of interest to be evolved encodes a protein. In some embodiments, the protein comprises one or more disulfide bonds. In some embodiments, the protein is an antibody, antibody fragment, or single-chain variable region (scFv), single-domain antibody, extracellular receptor, extracellular protease, monobody, adnectin, or nanobody.

In some embodiments, the protein further comprises a capture tag. In some embodiments, the capture tag comprises a peptide. In some embodiments, the capture tag comprises a SH2 domain or a GCN4 leucine zipper domain.

In some embodiments, the DNA binding protein is a bacterial DNA binding protein. In some embodiments, the bacterial DNA binding protein comprises a CadC protein (SEQ ID NO: 33) or a fragment thereof. In some embodiments, the DNA binding protein lacks a periplasmic sensor domain. In some embodiments, the DNA binding protein is encoded by the nucleic acid sequence set forth in SEQ ID NO: 11.

In some embodiments, the periplasmic capture agent comprises a cognate binding partner of the first gene product.

In some embodiments, the periplasmic capture agent comprises an antigen that binds the first gene product.

In some embodiments, the periplasmic capture agent comprises an antibody or fragment thereof that binds to the first gene product. In some embodiments, the periplasmic capture agent comprises a monobody that binds to the first gene product.

In some embodiments, the first expression construct further comprises a nucleic acid sequence encoding a portion of a split-intein. In some embodiments, the portion of the split-intein is connected to a portion of a periplasmic signal peptide sequence. In some embodiments, the portion of the periplasmic signal peptide sequence encodes amino acids 1-8 of SEQ ID NO: 32.

In some embodiments, the split-intein comprises a Nostoc punctiforme (Npu) trans-splicing DnaE intein N-terminal portion or C-terminal portion. In some embodiments, the split-intein is encoded by the nucleic acid sequence set forth in SEQ ID NO: 19.

In some embodiments, the selection phage further comprises a nucleic acid sequence encoding a portion of a split-intein connected to the gene of interest to be evolved. In some embodiments, the portion of the split-intein is connected to a portion of a periplasmic signal peptide sequence. In some embodiments, the portion of the periplasmic signal peptide sequence encodes amino acids 9-20 of SEQ ID NO: 32.

In some embodiments, the split-intein comprises a Nostoc punctiforme (Npu) trans-splicing DnaE intein N-terminal portion or C-terminal portion. N some embodiments, the split-intein is encoded by the nucleic acid sequence set forth in SEQ ID NO: 20.

In some embodiments, the conditional promoter comprises two or more DNA binding protein binding sites. In some embodiments, the two or more binding sites comprise a Cad1 binding site and a Cad2 binding site. In some embodiments, the conditional promoter comprises a P_{cadBA} promoter. In some embodiments, the conditional promoter comprises the sequence set forth in SEQ ID NO: 10.

In some embodiments, the vector system further comprises a mutagenesis plasmid. In some embodiments, the mutagenesis plasmid comprises a gene expression cassette encoding a mutagenesis-promoting gene product. In some embodiments, the expression cassette comprises a conditional promoter, the activity of which depends on the presence of an inducer. In some embodiments, the conditional promoter is an arabinose-inducible promoter and the inducer is arabinose.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF DRAWINGS

The following Drawings form part of the present Specification and are included to further demonstrate certain aspects of the present disclosure, which can be better understood by reference to one or more of these Drawings in combination with the Detailed Description of specific embodiments presented herein. For purposes of clarity, not every component may be labeled in every Drawing. It is to be understood that the data illustrated in the Drawings in no way limit the scope of the disclosure.

FIGS. 1A-1C. Periplasmic PACE (pPACE) selection system. FIG. 1A shows an overview of some embodiments of phage-assisted continuous evolution (PACE). Selection phage (SP) encode an evolving protein, in place of the native phage gene III (gIII), which encodes essential phage protein pIII. Host cells are transformed with a mutagenesis plasmid (MP) and one or more accessory plasmids (AP) encoding selection-specific genes. The selection links the desired function of the evolving protein to expression of gene III. Induction of the MP with arabinose rapidly mutates the evolving gene. Phage encoding functional variants of the evolving protein trigger gIII transcription and pIII translation, and are thus able to propagate in a fixed-volume “lagoon,” while phage with nonfunctional variants are diluted out of the lagoon over time. FIG. 1B shows native E. coli CadC signaling function. The CadC sensory domain dimerizes under conditions of high pH and low lysine concentration in the periplasm, leading to dimerization of the cytoplasmic component of CadC and activation of P_{cadBA}. FIG. 1C is a schematic describing some embodiments of periplasmic PACE (pPACE) methods. Phage encode an evolving protein (e.g., a single-chain variable fragment antibody) fused to a GCN4 leucine zipper. Following periplasmic export, GCN4 directs dimerization of the scFv-GCN4 species. Upon binding the target antigen, the dimeric scFv brings together two monomers of CadC linked to the antigen. Once in close proximity, the cytoplasmic DNA-binding domains of dimeric CadC cooperatively bind the DNA elements Cad1 and Cad2 of promoter P_{cadBA}, inducing transcription of gIII and phage propagation.

FIGS. 2A-2G. Periplasmic phage-assisted non-continuous evolution of the dimeric knottin YibK rescues binding mutants and evolves new disulfide bonds. FIG. 2A is a schematic of homodimeric YibK selection. HA4 monobody recruits SH2 to CadC, and CadC monomers are brought together by homodimerization of YibK. FIG. 2B shows a luminescence-based transcriptional activation assay comparing the performance of wild-type YibK-SH2 construct (WT) to the V139R binding mutant in the presence and absence of a signal sequence (SS) to direct periplasmic export (the architecture of the luciferase-based transcriptional reporter is shown in FIG. 18A). Bar values and error bars represent the mean and standard deviation (s.d.) of three independent biological replicates. FIG. 2C shows a phage propagation assay. Mid-log-phase cultures of selection strains were inoculated with phage and allowed to propagate overnight before determining titer. WT SS-YibK-SH2 phage enrich robustly, while the YibK V139R point mutant in the same construct enriches weakly, and phage encoding only SP-SH2 fail to enrich. Bar values and error bars represent the mean and s.d. of two independent biological replicate experiments carried out on separate days. FIG. 2D depicts phage-assisted noncontinuous evolution (PANCE) of YibK variant V139R evolves variants 3.6 and 3.7, showing two compensatory point mutations, A138D and R146C. R146C establishes a novel intermolecular disulfide bridge, resulting in a covalently bonded dimeric species which can be eliminated by addition of a reducing agent, as shown by Western blot of purified YibK protein (FIG. 2E; full gel image provided in FIG. 17B-17C). FIGS. 2F and 2G show that A138D restores wild-type activity in a V139R background in transcription assays (FIG. 2F), and likely forms a salt bridge with R139, as seen in the crystal structure of YibK dimer (FIG. 2G). Positions 138 and 139 are in contact at the dimer interface. PDB ID=1MXI. The experiment shown in FIG. 2E was repeated once with similar results. Bar values and error bars in FIG. 2F represent the mean and s.d. of three biological replicates.

FIGS. 3A-3F. Initial design of pPACE and mechanism of selection survival through homodimerization. FIG. 3A is a schematic overview of some embodiments of pPACE. FIG. 3B shows a luminescence-based transcriptional activation assay comparing the performance of Ω-graft (abbreviated Ω-g) to the L231F F232A (here abbreviated FA) binding mutant in the presence and absence of its cognate antigen, GCN4(7P14P) (abbreviated GCN4) in the system diagrammed in FIG. 3A. FIG. 3C shows that PACE generates multiple variants with spontaneous N-terminal or 4×GGGS (SEQ ID NO: 43) linker cysteine residues in addition to variants reversing mutation L231F (full results shown in FIG. 12B). FIG. 3D shows a transcriptional activation assay. In a non-binding background, N-terminal cysteines drive partial or complete restoration of P_{cadBA} transcriptional activation, indicating a mechanism of surviving the selection by formation of novel disulfide bonds that generate covalent homodimeric scFvs. as shown in FIG. 3E. Homodimeric scFv-SH2 fusions are able to drive CadC-HA4 dimerization without involvement of the antigen. Bar values and error bars in FIG. 3B and FIG. 3D represent the mean and s.d. of three independent biological replicates. FIG. 3F shows novel selection architecture designed to alleviate dimerization issues addressed above.

FIGS. 4A-4I. Second-generation pPACE selection reverts a binding mutant in Ω-graft scFv. FIG. 4A is a schematic of components expressed in periplasmic PACE to prevent selection survival from homodimerization of the protein of interest, instead of target binding. Ω-graft (Ω-g) scFvs form covalent dimers through N-terminal cysteine residues. GCN4 monomeric variant 7P14P is used to avoid dimerization of CadC in the absence of scFv:antigen binding. Promoter Pₚᵣₒ₃ is a low-level constitutive promoter. Pg_{III} is a native phage promoter. FIG. 4B shows an overnight phage propagation assay of Ω-graft scFv variant SP, illustrating the effect of the L231F mutation on phage propagation. Introduction of a stop codon into position 100 of the scFv construct (L231F-STOP) prevents phage propagation. Splitting the signal sequence using an intein (intein-L231/F) leads to reduced propagation. Bar values and error bars represent mean and s.d. of three biological replicate experiments conducted on separate days. FIG. 4C shows a plaque assay visualizing overnight expansion of intein-SS₉₋₂₀ phage variants L231 and L231F as in FIG. 4B (full plates are provided in FIGS. 13C-13D). FIG. 4D shows a Ω-graft selection overview. After periplasmic export and SS cleavage, a cysteine is exposed at the N-terminus to mediate covalent disulfide bonding of two scFv monomers. Binding of two GCN4 antigens by dimeric scFvs leads to activation of P_{cadBA}. FIGS. 4E-4F: PACE was carried out over 156 hours using full-length SS-scFv phage (FIG. 4E) or split intein SS-scFv phage (FIG. 4F). To impose additional challenges to the selection, full-length SS-scFv phage were also challenged to correct a nonsense mutation. By 96 hours, phage had converged upon solutions shown in FIG. 4G (as well as in FIGS. 10A-10B). Duplicates of each PACE experiment (not shown) were evolved with similar outcomes, correcting W100* and enriching F231L in the replicate of FIG. 4E and discovering L224S and F231L in replicate of FIG. 4F. FIG. 4H depicts a luminescence assay showing increased P_{cadBA} activation as a result of point mutation L224S in an L231F background. Bar values and error bars represent the mean and s.d. of three biological replicates. FIG. 4I illustrates a Western blot showing Ω-graft and L224S evolved mutant, expressed from P_{T7Lac} in BL21*D3 cells. The figure shows that L224S increases the solubility of Ω-graft scFv by roughly 8-fold. This experiment was repeated once with similar results (full gel and densitometry analyses provided in FIGS. 10C-10D and 11G).

FIGS. 5A-5I. Evolution of trastuzumab variants with improved binding to a Her2-mimetic peptide. FIG. 5A shows components of the second-generation periplasmic PACE system to evolve trastuzumab. The H98 peptide is a structural homologue of the Her2 epitope. A C-terminal dimeric GCN4 peptide directs dimerization of scFvs. FIG. 5B shows a phage propagation assay of starting genotypes and negative controls. Sequences with intein-split SS are indicated as ‘intein’. FIG. 5C shows that PACE was carried out over 120 hours using full-length (lagoons L1-L2) or split intein signal sequence (lagoon L3). By 96 hours, all three lagoons converged on discrete solutions, shown in FIG. 5D (also in FIG. 6A). FIG. 5E shows luminescence assay with trastuzumab (abbreviated TR) and evolved trastuzumab variants demonstrates increased P_{cadBA} activation. Luminescence/OD₆₀₀ values are shown relative to that of trastuzumab. FIG. 5F: ELISA shows modest improvement in binding. Values represent the mean and individual data points of four technical replicates from the same protein preparation (data points at far ends of the binding curve, used to verify top and bottom values, can be found in FIG. 6C. This experiment was repeated with four separate protein preparations and gave similar results. Average EC₅₀ and Hill slope values from all replicate experiments can be found in Table 1. PAGE analysis of purified protein used in this representative ELISA is shown in FIG. 20B). FIGS. 5G-5H illustrates Western blot and Coomassie-stained gels of TR and evolved variants expressed from the T_{7Lac} promoter in BL21*DE3 cells, showing improved soluble expression of variant 3.2 (full gels shown in FIGS. 11A-11B). Densitometry data reflects mean and s.d. of Western blot method and includes three independent biological replicates conducted on separate days. FIG. 5I shows the location of individual evolved mutations from PACE in the crystal structure of trastuzumab Fab bound to Her2 (PDB ID: 1N8Z). Bar values and error bars in FIG. 5B, FIG. 5E, and FIG. 5H represent the mean and s.d. of three independent biological replicates.

FIGS. 6A-6E. Periplasmic PACE of trastuzumab. FIG. 6A depicts individual phage emerging from PACE at 96 hours showing strong convergences of two distinct genotypes. The signal sequence (SS) directs periplasmic export of the scFv. FIG. 6B illustrates that ELISA shows no significant change in affinity of trastuzumab variants 1.1 and 3.2 for Her2 compared to trastuzumab (TR). Data reflect mean and s.d. of three technical replicates. This assay was repeated once with a separate protein preparation and yielded similar results. FIG. 6C shows full ELISA against mimetic peptide H98 described in (FIG. 5F), showing data points at far ends of the H98 dilution series. Four technical replicates are shown. This experiment was repeated three times with similar results. Mean IC₅₀ values and s.d. from all four experiments are provided in Table 1. FIG. 6D shows the crystal structure of trastuzumab fragment bound to Her2, showing the location of PACE-evolved mutations. Mutations are shown as spheres and are shaded as in FIG. 6A. FIG. 6E is a close-up of FIG. 6D, also showing residues N30 and T94. These residues are predicted to be directly involved in binding of the trastuzumab light chain to the Her2 mimetic peptide H98.

FIGS. 7A-7C. Trastuzumab and evolved variants require disulfides for activity. FIG. 7A depicts Coomassie gel showing purified trastuzumab (TR) scFv and evolved variants 1.1 and 3.2 with or without the addition of dithiothretiol (DTT) as a reducing agent. FIGS. 7B-7C depicts a luminescence-based transcriptional activation assay showing the impact of removing disulfides from trastuzumab and evolved variants by mutating four disulfide-forming Cys residues to Ser. Bars represent mean and s.d. from four biological replicates pooled from two separate experiments. Variants 1.1 and 3.2 have been removed in FIG. 7C to allow lower values to be compared.

FIGS. 8A-8H. Split-intein signal sequence allows regulation of antibody export to the periplasm. FIG. 8A shows a luminescence-based transcriptional activation assay to evaluate disruption of P_{CadBA} signaling caused by insertion into the signal sequence of the CFN scar that is the product of intein-mediated cleavage into the signal sequence. Residues CFN can be inserted into the SS between positions 8 and 9 without loss of periplasmic scFv-mediated transcriptional activation of P_{CadBA}. Bar values reflect mean and s.d. of three biological replicates. FIG. 8B depicts a signal sequence (SS) sequence (KQSTIALALLPLLFTPVTKA (SEQ ID NO: 32)), showing locations of intein insertion. FIGS. 8C-8D show selection phage enrichment assays. Intein NpuC domain is required for reconstitution of the SS and P_{CadBA}-gIII transcription activation (FIG. 8C). Likewise, gIII is not produced when fragment SS₁₋₈-NpuN is not supplied (FIG. 8D). Bar values and error bars reflect mean and s.d. of two or three biological replicates carried out on separate days. FIGS. 8E-8F show an overview of intein-mediated split SS system. SS residues 1-8 and Npu N-terminal domain are provided constitutively by an accessory plasmid (AP). FIGS. 8G-8H illustrate a Western blot of periplasmic extraction from BL21*DE3 cells, showing intein-mediated periplasmic scFv expression. Expression of SS₁₋₈-NpuN was driven by arabinose-inducible promoter P_{BAD} and induced with multiple concentrations of arabinose, with a constant level of IPTG (0.1 mM) inducing expression of NpuC-SS₉₋₂₀-scFv. ScFv with full-length SS was used as a positive control. Single transformants, lacking plasmids encoding either SS₁₋₈-NpuN or NpuC-SS₉₋₂₀-scFv, were used as negative controls. Concentrations are shown in FIG. 8H corresponding with lanes in FIG. 8G. Maltose-binding protein (MBP) was used as a periplasmic fraction loading control. Results of three replicates carried out on separate days were quantified by densitometry and were normalized first to the loading control, then to the value of positive control SS-scFv. Bar values and error bars reflect mean and s.d. of three biological replicates carried out on separate days.

FIGS. 9A-9B. Microscale thermophoresis analysis of trastuzumab scFv (TR) and variants 1.1 and 3.2. FIG. 9A shows MST raw data traces representing three technical replicates per sample. FIG. 9B shows calculated binding curves and individual data points for all replicates. One TR data point, shown in grey, was omitted from the analysis as an outlier due to evidence of fluorophore adsorption.

FIGS. 10A-10E. Second-generation periplasmic PACE of the Ω-graft antibody. FIGS. 10A-10B show Ω-graft (Ω-g) selection phage sequences showing convergent evolution of mutations during PACE. Use of full-length SS (3705c) appears to select solely for correction of the stop codon and L231F binding mutant, while use of a split intein SS (40o4c) selects for correction of both the binding mutant and L224S. The roles of I3N and L48V were not characterized. A single replicate of each population also enriched 100W and 231L (replicate of FIG. 10A) or 224S and 231L (replicate of FIG. 10B). FIG. 10C shows a full Western blot from FIG. 4I showing the effect of mutation L224S on soluble and insoluble expression levels across multiple IPTG concentrations when scFvs are expressed from P_{T7Lac} in BL21*DE3 cells.

FIG. 10D shows gel densitometry quantification of bands in FIG. 10C and in an additional biological replicate experiment carried out on a separate day. normalized to GroEL reference. The value for the variant 2.8 (L224S) band was then normalized again to the value for Ω-g. FIG. 10E shows additional Western blot data showing expression of Ω-g and variant 2.8 from the IPTG-inducible promoter P_{T7Lac} in BL21*DE3 cells, at multiple levels of induction with IPTG, including untransformed as well as uninduced controls.

FIGS. 11A-11G. Soluble expression and thermostability characteristics of evolved trastuzumab variants 1.1 and 3.2. FIGS. 11A-11B illustrate full Western blot and Coomassie gel of TR and evolved variants expressed from P_{T7Lac} in BL21*DE3 cells, shown in FIG. 5G. FIGS. 11C-11D show relative expression levels of trastuzumab variants 1.1 and 3.2 as determined by gel densitometry in Western blotting (FIG. 11C) and in Coomassie-stained SDS-PAGE gel (FIG. 11D). Band intensities are normalized first to a reference band, then to band intensity of unmodified trastuzumab. Two to three replicate experiments conducted on separate days and with fresh transformations of BL21*DE3 cells are shown for each. FIG. 11E shows SDS-PAGE of purified trastuzumab (TR) and variants 1.1 and 3.2 expressed in BL21*DE3 cells at 16° C. A BSA standard is also shown. These samples were used in diluted form in representative ELISA and MST data (FIG. 5F, Table 1, FIG. 6B-6C). The Coomassie-stained SDS-PAGE gel showing diluted samples can be found in FIG. 20B. FIG. 11F shows melting temperature curves of trastuzumab scFv and evolved variants. Data reflects individual data points, mean and s.d. of pooled data from experiments conducted with separate protein preparations and on separate days, each with four technical replicates. Purified protein used in both replicates can be found in FIG. 20C. FIG. 11G illustrates an additional Western blot showing two levels of expression of TR an evolved variants from the IPTG-inducible T_{7Lac} promoter in BL21*DE3 cells, as well as untransformed controls.

FIGS. 12A-12B. Characterization of the initial pPACE system. Ω-graft (Ω-g)-SH2 phage evolution in original selection architecture. FIG. 12A shows restriction-enzyme-mediated characterization of monoclonal phage (lanes 2-3) and PANCE and PACE outputs. In these selections, no mutagenesis was induced, and phage populations were seeded with binding mutant L231F F232A and unmodified Ω-g in the indicated ratios. PANCE was passaged by 1:100 dilution of phage. HinfI (5′-G^{∨}ANTC) cleaves the gene encoding the L231F F232A Ω-graft mutant (5′-GG^{∨}ATTCGCT), resulting in cleavage of 430-bp band into 280-bp and 150-bp bands, but does not cleave the unmodified Ω-graft sequence (5′-GGACTTTTT). FIG. 12B shows phage Ω-graft sequences resulting from PACE with mutagenesis showing mutations to cysteine at the N-terminus (position R1 following the cleaved signal sequence) and linker (position G119), and poor enrichment of the F231L reversion.

FIGS. 13A-13G. Characterization of second-generation pPACE system. FIGS. 13A-13D depict phage enrichment assays showing stringency parameters of various accessory plasmid (e.g., AP1) constructs. Each quadrant represents 10 μL undiluted selection phage (SP) enriched overnight on the indicated AP1. AP1 constructs differ by strength of ribosome-binding site (RBS) directing gene III transcription from P_{CadBA}. All phage contain the pre-encoded R1C mutation to direct covalent dimerization. Phage with the 3705c construct design have full-length signal sequence (SS), while those with the 4004c construct have the split-intein signal sequence (SS). Phages are visible as dark spots. FIG. 13E depicts a luciferase-based transcriptional activation assay showing that L231F is responsible for loss of binding in the L231F F232A mutant. Bar values and error bars reflect the mean and s.d. of three biological replicates. FIG. 13F shows relative strengths of AP1 constructs as measured by relative enrichment of phage 3705c variant 1.1 (L231). Enrichment values are normalized to AP1 construct pMM116a1, which encodes an sd8 RBS and represents an enrichment score of 1. Bar values and error bars represent mean and s.d. of three biological replicates carried out on separate days. FIG. 13G shows a table summarizing the results shown in FIGS. 13A-13D including phage construct genotypes.

FIGS. 14A-14B. Design of periplasmic PACE of trastuzumab scFv. FIG. 14A shows a phage enrichment assay evaluating domains to direct the dimerization of anti-HER2 antibody trastuzumab (TR). Dimerization with YibK imposes a fitness cost to phage when compared to dimerization with GCN4, likely due to its larger size. Bar values and error bars represent mean and s.d. of two or three biological replicates conducted on separate days. FIG. 14B shows an overview of trastuzumab periplasmic selection.

FIGS. 15A-15C. Periplasmic PACE of trastuzumab scFv at high stringency produces no novel converged mutations. FIG. 15A shows periplasmic PACE selection with increased stringency, seeded from lagoons 1 and 3 of trastuzumab scFv at 120 hours. On AP1, ribosome binding site (RBS; see arrow) strength driving pIII translation has been reduced from sd2 (0.001 relative expression units compared to SD8) to sd2G (0.0004 relative expression units compared to SD8). This change is expected to increase overall selection pressure. On AP2, constitutive promoter Pₚᵣₒ₃ (0.017 relative promoter units compared to promoter P_{ProD}) has been replaced with constitutive promoter Pₚᵣₒ₁ (0.009 relative promoter units compared to promoter P_{ProD}). This change is expected to reduce antigen availability of CadC-H98 and increase selective pressure for high affinity to H98. FIG. 15B shows trastuzumab scFv pPACE carrying populations L1 and L3 (FIG. 5, FIGS. 6A-6E) forward from 120 h timepoint into the more stringent selection conditions shown in FIG. 15A. Drift was applied from 120 h to 168 h. resulting in a period of low selective pressure to increase the size of the scFv library available for selection. FIG. 15C depicts that individual phage emerging from high-stringency pPACE of trastuzumab scFv at 256 hours show a lack of converged mutations that were not present in hours 1-120 of pPACE experiment (FIG. 5, FIGS. 6A-6E). Each evolution was repeated once with similar results.

FIGS. 16A-16D. PANCE of YibK. FIG. 16A show a periplasmic PACE circuit to correct monomeric binding mutant in YibK. The SH2-binding HA4 monobody is used to recruit the SH2-fused YibK species to CadC. FIG. 16B show phage titers through 24-hour cycles of PANCE. FIG. 16C depicts positions mutated in YibK PANCE shown in the YibK dimer crystal structure (PDB ID: 1J85). Positions are colored to correspond to YibK variant sequences shown in FIG. 16D. Position R146 is in close proximity to R146′ on the opposing subunit, while positions A138 and V139 make mutual contacts (A138:V139′. A138′:V139) at the dimer interface. Position V159, which falls in an unstructured region not captured by the crystal structure, is not shown.

FIGS. 17A-17E. Western blots show YibK-SH2 periplasmic localization and disulfide-mediated covalent bond formation. FIG. 17A show periplasmic extraction following arabinose (abbreviated Ara) induction of YibK-SH2 expression from P_{BAD}. FIG. 17B shows a Coomassie-stained gel of IMAC-purified 6×His-tagged YibK (21.6 kDa). The covalent dimeric species (43 kDa) is visible for the V139R R146C variant and is destroyed by addition of a reducing agent, dithiothreitol (DTT). FIG. 17C illustrates a full Western blot from FIG. 2E showing purified YibK protein as in FIG. 17B. The 43 kDa band representing the dimeric species is visible. FIG. 17D illustrates a full Western blot of whole-cell lysate showing a 60-kDa band representing a covalent YibK-SH2 dimer that is dependent on mutation R146C. and that is destroyed by the addition of DTT. The monomeric YibK-SH2 construct is 30 kDa. FIG. 17E illustrates a Western blot from FIG. 17D with GroEL (57 kDa) reference channel hidden, to better reveal 60 Da band.

FIGS. 18A-18C. Effect of host cadCBA operon deletion. FIG. 18A shows an overview of the CadC luciferase-based transcriptional activation reporter of YibK dimerization. The monobody HA4 binds and recruits SH2 with high affinity. FIG. 18B depicts a phage-induced luciferase transcriptional activation time course in unmodified host strain S2060, which shows background signaling mediated by wild-type M13 phage infection (no YibK expression). A single replicate is shown. This assay was repeated once with similar results. FIG. 18C shows a phage-induced luciferase transcriptional activation time course in a PACE host strain with deletion of the native cadCBA operon, which shows no M13-mediated background P_{CadBA} signaling. ‘Neg’ indicates monomeric YibK mutant V139R. Data reflect mean and s.d. of three biological replicates. Individual data points are also shown.

FIGS. 19A-19B. Optimization of P_{CadBA}. FIG. 19A shows an overview of CadC luciferase-based transcriptional activation reporter. FIG. 19B shows P_{CadBA} optimization. AP1 constructs incorporate three different spans of upstream untranslated regions of P_{CadBA}, which is activated by CadC dimerization. CadC molecules bind Cad1 and Cad2 DNA motifs at positions −144 to −112 bp and −89 to −59 bp respectively, but retention of 5′ UTR up to base-600 leads to maximal signal-to-noise ratio across multiple levels of arabinose-mediated P_{BAD} induction of CadC-GCN4. Y-axis shows the ratio of OD₆₀₀-normalized luminescence induced by wild-type GCN4 leucine zipper to OD₆₀-normalized luminescence induced by GCN4 monomeric variant 7P14P. Bar values and error bars represent mean and s.d. of two biological replicates.

FIGS. 20A-20C. Trastuzumab scFv and evolved variants used in biochemical characterizations. FIG. 20A shows initial protein purification, and FIG. 20B shows 25 μg/mL dilution of trastuzumab scFv and variants 1.1 and 3.2 used in MST and representative ELISA experiments (FIG. 5F, Table 1, FIGS. 9A-9B, FIGS. 6B-6C). FIG. 20A is identical to FIG. 11E; shown again here for comparison. FIG. 20C shows two replicate protein purifications used in thermal melt experiments (Table 1, FIG. 11F). BSA standards also shown.

FIG. 21A-21E show data relating to evolution of the ciA-C2 single-domain (VHH) antibody to bind BoNT/A receptor-binding domain. FIG. 21A shows a schematic depicting one embodiment of a selection architecture. The VHH is expressed as a fusion with an SH2ABL domain (here simplified to SH2) and is recruited to CadC through binding to the monobody HA4. The antigen is expressed as a CadC fusion, creating an asymmetric CadC dimer upon binding. FIG. 21B shows PACE selection in two legs with increasing stringency. Drift was applied for the first 24 hours of each leg of PACE. In the second leg of PACE, lagoons L1-L4 were seeded with single selection phage from the final timepoint of the previous leg of PACE. FIG. 21C shows genotypes of sequenced selection phage from PACE endpoints (292 hours total evolution). Four phage per lagoon were sequenced. e.g. variants 292.1.1-4 from lagoon L1, variants 292.2.1-4 from lagoon L2, etc. FIG. 21D shows location of specific point mutations isolated in PACE shown in the crystal structure of ciA-C2 bound to the BoNT/A receptor-binding domain. Mutated residues are shown as spheres. Spheres in the center indicate BoNT/A residue N905. FIG. 21E show binding data for several ciA-C2 variants, with combinations of mutations identified by PACE, to BoNT/A RBD, measured by luciferase-based transcriptional assay.

FIG. 22. Selection architecture for serine protease evolution using periplasmic PACE. Two SH2_{ABL} domains (here simplified to SH2) are tethered together by a linker containing a substrate sequence that is not desirable as a serine protease cleavage target. Both domains are further tethered to a degron tag by a second linker containing a desired target sequence. Cleavage of the desired sequence by the evolving protease removes the degron tag, rescuing the linked SH2 domains from degradation by host periplasmic proteases. Cleavage of the undesired substrate separates the two SH2 domains, leading to binding of CadC monomers which not only fails to drive CadC dimerization, but also competes with intact SH2-SH2 fusion proteins for binding of HA4 domains.

FIGS. 23A-23B. Phage-based and plasmid-based periplasmic scFv expression does not impair host cell growth rate. FIG. 23A shows results of a time growth assay measuring OD₆₀₀ of host cells transformed with accessory plasmid pJC175e, which provides free pIII and allows selection-independent phage propagation, grown in the presence of two initial titers of selection or control phage. Three biological replicates are shown. FIG. 23B shows results of a time-course growth assay measuring OD₆₀₀ of host cells with plasmid-based expression of trastuzumab scFv under an arabinose-driven promoter. Arabinose concentrations are indicated in the figure legend (0 um, 100 um, 500 uM, or 1000 uM). Three biological replicates are shown. Points represent individual data, while lines indicate mean values.

### Definitions

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Ser. No. 13/922,812, filed Jun. 20, 2013; U.S. Application, U.S. Ser. No. 62/067,194, filed Oct. 22, 2014, U.S. Pat. No. 9,023,594, issued May 5, 2015, and International PCT Application, PCT/US2018/051557, published as WO 2018/056002 on Mar. 21, 2019, the entire contents of each of which is incorporated herein by reference.

The term “promoter,” as used herein, refers to a nucleotide sequence capable of controlling the expression of a coding sequence or functional nucleic acid. In general, a nucleic acid sequence encoding a gene product is located 3′ of a promoter sequence. In some embodiments, a promoter sequence consists of proximal and more distal upstream elements and can comprise an enhancer element.

The term “periplasmic space” or “periplasm,” as used herein, refers to the space between the inner and outer membrane in Gram-negative bacteria and/or the space found between the inner membrane and the peptidoglycan layer. The term may also be used to refer to the intermembrane spaces of fungi and organelles. The matrix contained in the periplasmic space is referred to as the “periplasm” and is gel like in composition. The periplasm is known for containing multiple enzymes, including, but not limited to, alkaline phosphatases, cyclic phosphodiesterases, acid phosphatases, and 5′-nucleotidases. With a redox potential higher than that of the cytoplasm (−165 mV vs −260/−280 mV in E. coli, respectively), the periplasmic space is considered as an oxidizing compartment. Consistently, the majority of cysteine residues present in periplasmic proteins are oxidized to disulfides. These disulfides, which are important for protein stability, are introduced in periplasmic proteins by the soluble oxidoreductase DsbA, a thioredoxin-fold protein with a CXXC catalytic site. The cysteine residues of this conserved motif form a very unstable disulfide, which is transferred to newly synthesized proteins as they enter the periplasm, releasing DsbA in the reduced state. DsbA is then recycled back to the oxidized state by the IM protein DsbB, which generates disulfide bonds de novo from quinone reduction. DsbA preferentially introduces disulfides into proteins entering the periplasm by oxidizing cysteine residues that are consecutive in the protein sequence. (Isabelle S. Arts, Alexandra Gennaris, Jean-François Collet, Reducing systems protecting the bacterial cell envelope from oxidative damage, FEBS Letters, Volume 589, Issue 14, 2015, Pages 1559-1568). In some embodiments, a non-reducing environment is a periplasmic space. In some embodiments, a periplasmic space is a non-reducing environment.

The term “monobody.” as used herein, refers to synthetic binding proteins based on a molecular scaffold composed of a fibronection type III domain (FN3). Monobodies are considered to belong to a class of molecules called antibody mimics, and to be alternatives to traditional antibodies. They are typically highly specific for their targets and can be produced from libraries with diversified portions of the FN3 scaffold and mixes of amino acids using phage display or yeast surface display methods. The scaffold is often less than 90 residues permitting expression by transfecting a cell with a monobody expression vector.

The term “proximal,” as used herein, refers to a distance inside of which the two or more components which are described as being proximal affect one another (e.g., affect the activity of one another). For example, without limitation, in instances where two binding motifs are described as being proximal to one another, it shall be understood that the binding of one or the other may not initiate activity without the binding of the other and within a relative distance to one another. This may be, for example, because they are activated by a specific protein or pair of proteins (e.g., dimers) and are not intended to be activated in the absence of such specific protein or one portion of the dimer. In some embodiments, proximal means within (e.g., less than) 1,000 (e.g., 1,000, 900, 800, 700, 600, 500, 499, 498, 497, 496, 495, 494, 493, 492, 491, 490, 489, 488, 487, 486, 485, 484, 483, 482, 481, 480, 479, 478, 477, 476, 475, 474, 473, 472, 471, 470, 469, 468, 467, 466, 465, 464, 463, 462, 461, 460, 459, 458, 457, 456, 455, 454, 453, 452, 451, 450, 449, 448, 447, 446, 445, 444, 443, 442, 441, 440, 439, 438, 437, 436, 435, 434, 433, 432, 431, 430, 429, 428, 427, 426, 425, 424, 423, 422, 421, 420, 419, 418, 417, 416, 415, 414, 413, 412, 411, 410, 409, 408, 407, 406, 405, 404, 403, 402, 401, 400, 399, 398, 397, 396, 395, 394, 393, 392, 391, 390, 389, 388, 387, 386, 385, 384, 383, 382, 381, 380, 379, 378, 377, 376, 375, 374, 373, 372, 371, 370, 369, 368, 367, 366, 365, 364, 363, 362, 361, 360, 359, 358, 357, 356, 355, 354, 353, 352, 351, 350, 349, 348, 347, 346, 345, 344, 343, 342, 341, 340, 339, 338, 337, 336, 335, 334, 333, 332, 331, 330, 329, 328, 327, 326, 325, 324, 323, 322, 321, 320, 319, 318, 317, 316, 315, 314, 313, 312, 311, 310, 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273, 272, 271, 270, 269, 268, 267, 266, 265, 264, 263, 262, 261, 260, 259, 258, 257, 256, 255, 254, 253, 252, 251, 250, 249, 248, 247, 246, 245, 244, 243, 242, 241, 240, 239, 238, 237, 236, 235, 234, 233, 232, 231, 230, 229, 228, 227, 226, 225, 224, 223, 222, 221, 220, 219, 218, 217, 216, 215, 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1) nucleotides. In some embodiments, proximal means within (e.g., less than) 500. In some embodiments, proximal means within (e.g., less than) 400. In some embodiments, proximal means within (e.g., less than) 300. In some embodiments, proximal means within (e.g., less than) 200. In some embodiments, proximal means within (e.g., less than) 100. In some embodiments, proximal means within (e.g., less than) 50. In some embodiments, proximal means within (e.g., less than) 40. In some embodiments, proximal means within (e.g., less than) 30. In some embodiments, proximal means within (e.g., less than) 20. In some embodiments, proximal means within (e.g., less than) 10.

The term “continuous evolution,” as used herein, refers to an evolution process, in which a population of nucleic acids encoding a gene to be evolved (e.g., gene of interest) is subjected to multiple rounds of (a) replication, (b) mutation, and (c) selection to produce a desired evolved version of the gene that is different from the original version of the gene, for example, in that a gene product, such as, e.g., an RNA or protein encoded by the gene, exhibits a new activity not present in the original version of the gene product, or in that an activity of a gene product encoded by the original gene to be evolved is modulated (increased or decreased). The multiple rounds can be performed without investigator intervention, and the steps (a)-(c) can be carried out simultaneously. Typically, the evolution procedure is carried out in vitro, for example, using cells in culture as host cells. In general, a continuous evolution process provided herein relies on a system in which a gene encoding a gene product of interest is provided in a nucleic acid vector that undergoes a life-cycle including replication in a host cell and transfer to another host cell, wherein a critical component of the life-cycle is deactivated (e.g., production of pIII) and reactivation of the component is dependent upon an activity of the gene to be evolved that is a result of a mutation in the nucleic acid vector.

The term “vector,” as used herein, refers to a nucleic acid that can be modified to encode a gene of interest and that is able to enter into a host cell, mutate, and replicate within the host cell, and then transfer a replicated form of the vector into another host cell. Exemplary suitable vectors include viral vectors, such as retroviral vectors or bacteriophages, and conjugative plasmids. Additional suitable vectors will be apparent to those of skill in the art based on the instant disclosure.

The term “viral vector,” as used herein, refers to a nucleic acid comprising a viral genome that, when introduced into a suitable host cell, can be replicated and packaged into viral particles able to transfer the viral genome into another host cell. The term viral vector extends to vectors comprising truncated or partial viral genomes. For example, in some embodiments, a viral vector is provided that lacks a gene encoding a protein essential for the generation of infectious viral particles (e.g., pIII). In suitable host cells, for example, host cells comprising the lacking gene under the control of a conditional promoter, however, such truncated viral vectors can replicate and generate viral particles able to transfer the truncated viral genome into another host cell. In some embodiments, the viral vector is a phage, for example, a filamentous phage (e.g., an M13 phage). In some embodiments, a viral vector, for example, a phage vector, is provided that comprises a gene of interest to be evolved.

The term “phage,” as used herein interchangeably with the term “bacteriophage.” refers to a virus that infects bacterial cells. Typically, phages consist of an outer protein capsid enclosing genetic material. The genetic material can be single-stranded RNA (ssRNA), double-stranded RNA (dsRNA), single-stranded DNA (ssDNA), or double-stranded DNA (dsDNA), in either linear or circular form. Phages and phage vectors are well known to those of skill in the art and non-limiting examples of phages that are useful for carrying out the methods provided herein are λ (Lysogen), T2, T4, T7, T12, R17, M13, MS2, G4, P1, P2, P4, Phi X174, N4, Ø6, and @29. In certain embodiments, the phage utilized in the present invention is M13. Additional suitable phages and host cells will be apparent to those of skill in the art and the invention is not limited in this aspect. For an exemplary description of additional suitable phages and host cells, see Elizabeth Kutter and Alexander Sulakvelidze: Bacteriophages: Biology and Applications. CRC Press; 1st edition (December 2004), ISBN: 0849313368; Martha R. J. Clokic and Andrew M. Kropinski: Bacteriophages: Methods and Protocols, Volume 1: Isolation, Characterization, and Interactions (Methods in Molecular Biology) Humana Press; 1st edition (December 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1st edition (December 2008), ISBN: 1603275649; all of which are incorporated herein in their entirety by reference for disclosure of suitable phages and host cells as well as methods and protocols for isolation, culture, and manipulation of such phages).

The term “accessory plasmid,” as used herein, refers to a plasmid comprising a gene required for the generation of infectious viral particles under the control of a conditional promoter. In the context of the continuous evolution of a gene, transcription from the conditional promoter of the accessory plasmid is typically activated, directly or indirectly, by a function of the gene to be evolved. Accordingly, an accessory plasmid serves the function of conveying a competitive advantage to those viral vectors in a given population of viral vectors that carry a version of the gene to be evolved able to activate the conditional promoter or able to activate the conditional promoter more strongly than other versions of the gene to be evolved. In some embodiments, only viral vectors carrying an “activating” version of the gene to be evolved will be able to induce expression of the gene required to generate infectious viral particles in the host cell, and, thus, allow for packaging and propagation of the viral genome in the flow of host cells. Vectors carrying non-activating versions of the gene to be evolved, on the other hand, will not induce expression of the gene required to generate infectious viral vectors, and, thus, will not be packaged into viral particles that can infect fresh host cells.

The term “helper phage,” as used herein interchangeable with the terms “helper phagemid” and “helper plasmid,” refers to a nucleic acid construct comprising a phage gene required for the phage life cycle, or a plurality of such genes, but lacking a structural element required for genome packaging into a phage particle. For example, a helper phage may provide a wild-type phage genome lacking a phage origin of replication. In some embodiments, a helper phage is provided that comprises a gene required for the generation of phage particles, but lacks a gene required for the generation of infectious particles, for example, a full-length pIII gene. In some embodiments, the helper phage provides only some, but not all, genes for the generation of infectious phage particles. Helper phages are useful to allow modified phages that lack a gene for the generation of infectious phage particles to complete the phage life cycle in a host cell. Typically, a helper phage will comprise the genes for the generation of infectious phage particles that are lacking in the phage genome, thus complementing the phage genome. In the continuous evolution context, the helper phage typically complements the selection phage, but both lack a phage gene required for the production of infectious phage particles.

The term “selection phage,” as used herein interchangeably with the term “selection plasmid,” refers to a modified phage that comprises a gene of interest to be evolved and lacks a full-length gene encoding a protein required for the generation of infectious phage particles. For example, some M13 selection phages provided herein comprise a nucleic acid sequence encoding a gene to be evolved, e.g., under the control of an P_{cadBA} promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some selection phages provided herein comprise a nucleic acid sequence encoding a gene to be evolved, e.g., under the control of an P_{cadBA} promoter, and lack all or part of a gene encoding a protein required for the generation of infective phage particles, e.g., the gIII gene encoding the pIII protein.

The term “mutagenesis plasmid,” as used herein, refers to a plasmid comprising a gene encoding a gene product that acts as a mutagen. In some embodiments, the gene encodes a DNA polymerase lacking a proofreading capability. In some embodiments, the gene is a gene involved in the bacterial SOS stress response, for example, a UmuC, UmuD′, or RecA gene. In some embodiments, the gene is a GATC methylase gene, for example, a deoxyadenosine methylase (dam methylase) gene. In some embodiments, the gene is involved in binding of hemimethylated GATC sequences, for example, a seqA gene. In some embodiments, the gene is involved with repression of mutagenic nucleobase export, for example emrR. In some embodiments, the gene is involved with inhibition of uracil DNA-glycosylase, for example a Uracil Glycosylase Inhibitor (ugi) gene. In some embodiments, the gene is involved with deamination of cytidine (e.g., a cytidine deaminase from Petromyzon marinus), for example, cytidine deaminase 1 (CDA1). Mutagenesis plasmids (also referred to as mutagenesis constructs) are described, for example by International Patent Application, PCT/US2016/027795, filed Apr. 16, 2016, published as WO2016/168631 on Oct. 20, 2016, the entire contents of which are incorporated herein by reference.

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

The term “protein,” as used herein, refers to a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain; see, for example, cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these.

The term “gene of interest” or “gene to be evolved,” as used herein, refers to a nucleic acid construct comprising a nucleotide sequence encoding a gene product, e.g., an RNA or a protein, to be evolved in a continuous evolution process as provided herein. The term includes any variations of a gene of interest that are the result of a continuous evolution process according to methods provided herein. For example, in some embodiments, a gene of interest is a nucleic acid construct comprising a nucleotide sequence encoding an RNA or protein to be evolved, cloned into a viral vector, for example, a phage genome, so that the expression of the encoding sequence is under the control of one or more promoters in the viral genome. In other embodiments, a gene of interest is a nucleic acid construct comprising a nucleotide sequence encoding an RNA or protein to be evolved and a promoter operably linked to the encoding sequence. When cloned into a viral vector, for example, a phage genome, the expression of the encoding sequence of such genes of interest is under the control of the heterologous promoter and, in some embodiments, may also be influenced by one or more promoters comprised in the viral genome. In some embodiments, the term “gene of interest” or “gene to be evolved” refers to a nucleic acid sequence encoding a gene product to be evolved, without any additional sequences. In some embodiments, the term also embraces additional sequences associated with the encoding sequence, such as, for example, intron, promoter, enhancer, polyadenylation, and/or signal sequences (e.g., periplasmic signal sequences).

The term “evolved protein,” as used herein, refers to a protein variant that is expressed by a gene of interest that has been subjected to continuous evolution, such as PACE.

The term “host cell,” as used herein, refers to a cell that can host, replicate, and transfer a phage vector useful for a continuous evolution process as provided herein. In embodiments where the vector is a viral vector, a suitable host cell is a cell that can be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect.

The term “periplasmic capture agent.” as used herein, refers to an agent, for example, a nucleic acid, peptide, or protein, that functions to bind to a gene product (e.g., protein, peptide, etc.) expressed by a gene of interest in the periplasmic space of a cell (e.g., bacterial cell). Examples of periplasmic capture agents include, but are not limited to, antigens, antibodies or fragments thereof, single-chain variable regions (scFvs), monobodies, cognate binding partners (e.g., a ligand that binds to one or more specific receptors), etc. In some embodiments, a periplasmic capture agent comprises a periplasmic signal transduction signal peptide, or another signal peptide or sequence that directs translocation of the periplasmic capture agent into the periplasm of the cell.

### DETAILED DESCRIPTION

Aspects of the disclosure relate to compositions, methods, systems, uses, and kits for evolving proteins. The disclosure is based, in part, on the binding of a phage-expressed gene product of interest to a capture agent (e.g., a periplasmic capture agent) in the periplasmic space of bacteria, which in turn activates a conditional promoter to express a gene that is required for production of infectious phage. Expression of evolving proteins in the periplasm permits disulfide bond formation while retaining the protein being evolved within the bacterial host cell. Linking a protein's desired activity in the periplasm to phage propagation enables the continuous evolution of proteins that require a non-reducing environment to function. Without wishing to be bound by any particular theory, evolving genes of interest to function in the periplasmic space enables the production of proteins which require a non-reducing environment in order to fold and/or function properly.

Phage-assisted continuous evolution (PACE) can serve as a rapid, high-throughput system for evolving genes of interest. One advantage of the PACE technology is that both the time and human effort required to evolve a gene of interest are dramatically decreased as compared to conventional iterative evolution methods. During PACE, a phage vector carrying a gene encoding a gene of interest replicates in a flow of host cells through a fixed-volume vessel (a “lagoon”). For example, in some embodiments of PACE described herein, a population of bacteriophage vectors replicates in a continuous flow of bacterial host cells through the lagoon, wherein the flow rate of the host cells is adjusted so that the average time a host cell remains in the lagoon is shorter than the average time required for host cell division, but longer than the average life cycle of the vector, e.g., shorter than the average M13 bacteriophage life cycle. As a result, the population of vectors replicating in the lagoon can be varied by inducing mutations, and then enriching the population for desired variants by applying selective pressure, while the host cells do not effectively replicate in the lagoon.

Often, proteins (e.g., engineered proteins, wild-type proteins, etc.) have certain physiochemical properties, such as decreased stability (e.g., thermostability) and/or solubility that render them unsuitable for therapeutic or commercial use. Some aspects of this disclosure provide systems for improving the stability and/or solubility of proteins evolved during PACE. The systems, including recombinant expression constructs, also referred to as vectors if they are in the form of a plasmid, described herein can enhance selection of evolved proteins that are properly folded, have increased stability (e.g., thermodynamic stability), and/or solubility (e.g., enhanced soluble expression in bacteria, such as E. coli) while maintaining desired protein function.

Aspects of the disclosure relate to compositions (e.g., isolated nucleic acids and vectors) and methods for improving the activity, such as binding activity, enzymatic activity, etc. and/or the binding affinity (e.g., including but not limited to substrate specificity and/or affinity), stability, and/or solubility of proteins evolved using PACE. The disclosure is based in part on evolution of proteins carried out in the periplasm of a host cell (e.g., bacterial cell). In some embodiments, the evolution includes positive and negative selection systems that bias continuous evolution of a gene of interest towards production of evolved protein variants having desirable physiochemical characteristics, for example, increased, decreased, or new binding affinity, increased or decreased solubility, and/or increased or decreased stability (e.g., thermostability), altered substrate specificity, selectivity, or affinity, relative to a gene product of the gene of interest, such as a gene product that has not been evolved (e.g., subjected to PACE). Without wishing to be bound by any particular theory, selection constructs and systems described herein generally function by linking a desired physiochemical characteristic or function of an evolved protein to expression of a gene required for the generation of infectious viral particles (e.g., pIII), wherein the function occurs in a non-reducing environment.

Accordingly, in some aspects, the disclosure provides a method of continuous evolution comprising: (a) contacting a population of bacterial host cells in a culture medium with a population of selection phage comprising a gene of interest to be evolved and lacking a functional pIII gene required for the generation of infectious phage particles; wherein (1) the phage allow for expression of the gene of interest in the host cells; (2) the host cells are suitable host cells for phage infection, replication, and packaging, wherein the phage comprises all phage genes required for the generation of phage particles, except a full-length pIII gene; and (3) the host cells comprise: (i) a first expression construct encoding a fusion protein comprising a DNA binding protein connected to a periplasmic capture agent; and (ii) a second expression construct encoding a pIII protein under the control of a conditional promoter, wherein activation of the conditional promoter is dependent on binding of a first gene product of the gene of interest to the periplasmic capture agent; and (b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that are not infected by phage, wherein the binding of the first gene product to the periplasmic capture agent is a desired function, wherein phage expressing gene products having a desired function induce production of pIII and release progeny into the culture medium capable of infecting new host cells, and wherein phage expressing gene products having an undesired function do not produce pIII and release only non-infectious progeny into the culture medium.

As discussed elsewhere herein, the periplasm is an oxidizing environment. Such an environment does not negatively influence or inhibit the formation or stability of disulfide bridges, which inhibition can affect the activity of the gene product when active in alternative environments. Thus, by evaluating the activity of the gene product of the gene of interest in an environment which is more analogous to that of practical (e.g., clinical, environmental, diagnostic, therapeutic, etc.) use, translation of the evolved gene product from discovery to application is more likely.

Accordingly, aspects of the present disclosure relate to introducing genes of interest into a host cell by phage deficient in a gene product required for successful phage reproduction and packaging, directing gene products of the genes of interest thereof into the periplasm of a host cell where activity of the gene product modulates activation of expression of a gene required for phage reproduction in the host cell (e.g., pIII). As with traditional PACE directed evolution, the host cells contain the required element (e.g., gene product) to allow for successful propagation of the phage. The gene product, however, is under the control of a conditional promoter which is tied to the desired activity. Thus, only host cells infected by phage containing expression constructs encoding a gene product exhibiting the desired activity will activate expression in the host cell of the element needed for successful phage propagation (e.g., pIII). However, in the present disclosure, the desired activity is assessed and occurs in the periplasm of the host cell.

For example, in some embodiments, phage may comprise a first expression construct encoding a gene of interest. In some embodiments, a gene of interest encodes a first gene product. In some embodiments, a gene of interest may encode a protein for evolution.

In some embodiments, a host cell further comprises additional (e.g., 1, 2, 3, 4, 5, or more) expression constructs (e.g., plasmids, accessory plasmids) which encode for gene product (e.g., a second gene product) which is a target molecule for the first gene product. In arranging the PACE system as such, it is possible to tune the desired activity to focus on specific binding abilities (e.g., molecular recognition, antibody/antigen recognition, scFv/antigen recognition). For example, in some embodiments, a phage may introduce an expression construct for a scFv which is to be evolved to recognize (or increase/decrease recognition) a specific antigen. Such antigen (e.g., target molecule) may be expressed by the second expression construct. In some embodiments, a host cell comprises a second expression construct. In some embodiments, a second expression construct encodes a target molecule. In some embodiments, a target molecule comprises a recognition site for the first gene product. In some embodiments, a second expression construct is present on an accessory plasmid in a host cell. Binding, and binding abilities (e.g., molecular recognition, antibody/antigen recognition, scFv/antigen recognition, antibody/substrate affinity and/or specificity) may be based on any type of molecular binding, for example, without limitation, covalent bonding, non-covalent bonding, hydrophobic interactions, electrostatic interactions, hydrogen bonds, and/or Van der Waals forces. Such binding (e.g., affinity) may be measured by any means known to the skilled artisan, for example by measuring the dissociation constant.

In some embodiments, a gene product of any of the expression constructs disclosed herein, may encode gene products which naturally migrate, or locate, to the periplasm of a host cell. However, in many instances it may be helpful or necessary to incorporate elements which facilitate this migration e.g. to the periplasm. For instance, a gene of interest may encode a protein of interest for evolution as well as a signal peptide which has properties which give it an affinity for migration to the periplasm. These signals may be encoded to be attached to the protein of interest. Accordingly, in some embodiments, the gene of interest may further encode elements to facilitate migration or transfer of the protein into the periplasm of a host cell. For example, in some embodiments, a gene of interest may encode signal sequences (e.g., peptide sequences). In some embodiments, a gene of interest may encode a first gene product and a signal sequence. In some embodiments, a signal sequence is a signal sequence which facilitates entry to into the periplasm. In some embodiments, a signal sequence is a periplasmic signal sequence. In some embodiments, a signal sequence is attached to the N-terminus of a first gene product, or the C-terminus. In some embodiments, a signal sequence is derived from alkaline phosphatase A (PhoA), a periplasmic E. coli protein. In some embodiments, a signal sequence is a split intein sequence, as further defined herein. In some embodiments, where a signal sequence comprises, or is encoded as, a split intein, the portions (e.g., less than the whole) of the whole signal sequence may be attached to distinct gene products, which when reconstituted facilitate the migration of the entire construct into the periplasm. Alternatively, each split intein may migrate to the periplasm individually.

In some embodiments, a signal sequence comprises a nucleic acid sequence with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9%) identity to SEQ ID NO: 8-9. In some embodiments, a signal sequence comprises an nucleic acid sequence of SEQ ID NO: 8-9. The terms “percent identity,” “sequence identity,” “% identity.” “% sequence identity.” and % identical,” as they may be interchangeably used herein, refer to a quantitative measurement of the similarity between two sequences (e.g., nucleic acid or amino acid). The percent identity of genomic DNA sequence, intron and exon sequence, and nucleic acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

Some aspects of this invention provide a system for continuous evolution procedures, comprising of a viral vector, for example, a selection phage, comprising a multiple cloning site for insertion of a gene to be evolved, one or more additional accessory plasmids (e.g., comprising a selection system) as described herein, and, optionally, a mutagenesis expression construct. In some embodiments, a vector system for phage-based continuous directed evolution is provided that comprises (a) a selection phage comprising a multiple cloning site for insertion of a gene of interest to be evolved, wherein the phage genome is deficient in at least one gene required to generate infectious phage; (b) and at least one accessory plasmid comprising the at least one gene required to generate infectious phage particle under the control of a conditional promoter that is activated in response to a desired physiochemical characteristic (e.g., solubility, stability, etc.) and/or a desired activity of the gene to be evolved; and, optionally, (c) a mutagenesis expression construct as provided herein. In some embodiments, the host cell comprises additional expression constructs (e.g., plasmids, accessory plasmids) which encode mutagenic factors, e.g., gene products which effectuate mutagenesis. In some embodiments, the host cells are exposed to a mutagen. In some embodiments, the mutagen is ionizing radiation, ultraviolet radiation, base analogs, deaminating agents (e.g., nitrous acid), intercalating agents (e.g., ethidium bromide), alkylating agents (e.g., ethylnitrosourea), transposons, bromine, azide salts, psoralen, benzene,3-Chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl)acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide (captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-Aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9), or t-butyl hydroperoxide (BHP) (CAS no. 75-91-2).

In some embodiments, additional expression constructs are present in a host cell or phage (e.g., accessory plasmids). As can be envisioned by one of skill in the art, these accessory plasmids may be used to engineer or create a mechanistic environment which is conditionally activated by a desired activity. For example, in some embodiments, a phage may comprise an expression construct encoding a gene of interest (e.g., to express a gene product of interest (e.g., therapeutic protein, scFv), first gene product). The phage may further comprise an expression construct encoding additional accessory components, for example, linkers, signal sequences (e.g., periplasmic signal sequences), additional molecules (e.g., molecules to recognize monobodies or other elements of the system, e.g., SH2). Moreover, accessory plasmids may be present in the host cell which encode for proteins or molecules which are recognized by the first gene product, or which are desired to be recognized by the evolved gene product of the gene of interest. Accessory plasmids may further comprise expression constructs which encode for the element necessary for successful phage propagation which is missing from the phage genome (e.g., pIII). Accessory plasmids may further comprise sequences encoding elements necessary for recognition of the activity in the periplasm (e.g., CadC) and activation of promoter (e.g., P_{CadBA}) operably linked to the expression cassette of pIII. Further, accessory plasmids may comprise sequences which encode for gene products which when attached to CadC (e.g., monobodies) are recognized by elements attached to a first gene product and gene product which is desired to be recognized by the first gene product.

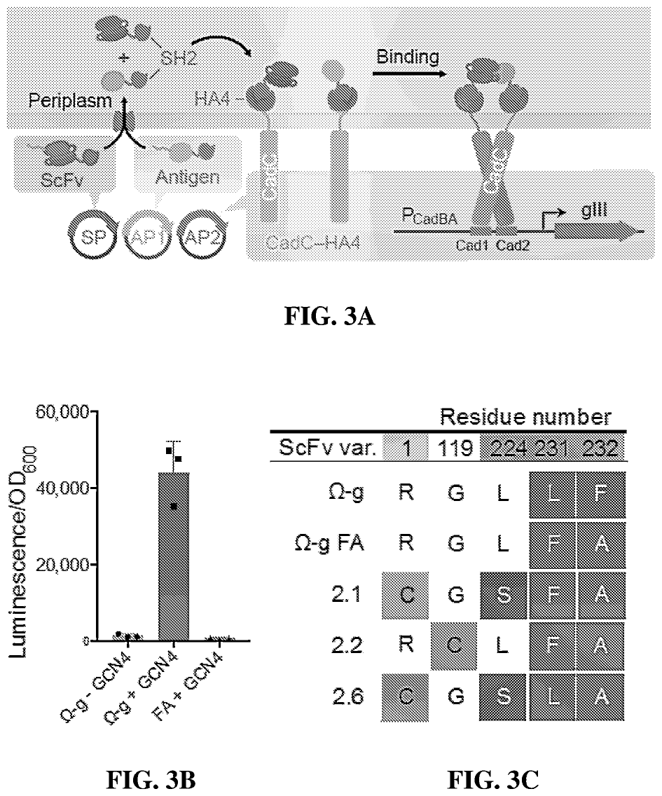

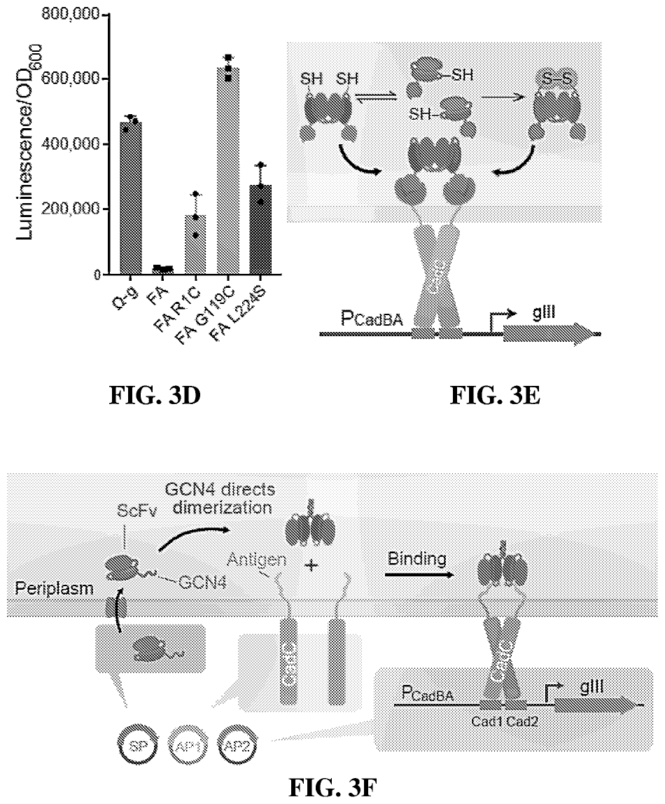

For example, what is described is a modular system (e.g., FIG. 3A), wherein the binding of gene products, expressed by the gene of interest and an additional expression construct (e.g., accessory plasmid) activates a conditional promoter. Further because each of these gene products (e.g., gene product of the gene of interest and an additional expression construct) may be attached to a periplasmic signal sequence, such gene products migrate to the periplasm. Moreover, as each gene product may comprise an additional element (e.g., SH2) which recognizes a monobody (e.g., HA4), when the gene products recognize one another, bind in the periplasm, they draw elements attached to them into close proximity. This proximity extends to the elements attached to a monobody (e.g., HA4), for example, CadC which then dimerizes. The resulting homodimer may then activate a promoter (e.g., P_{CadBA}), which may comprise DNA binding motifs such as Cad1 and Cad2 and drives expression of the element necessary for successful phage propagation (e.g., pIII).

In some embodiments, directed evolution as described herein uses any of the selection systems, nucleic acids, vectors (e.g., plasmids), apparatuses, and/or expression constructs as described herein.

### Selection Phages

Aspects of the disclosure relate to selection phages (SP) that encode one or more genes of interest to be evolved. A gene to be evolved may encode one or more gene products, for example, a peptide, protein, polypeptide, protein complex (e.g., one or more subunits of a protein complex), etc. In some embodiments, a gene of interest to be evolved encodes a protein, for example, a therapeutic protein. In some embodiments, the protein encoded by the gene of interest requires (or benefits from) a non-reducing environment, such as the periplasmic space of a bacterial cell, in order to fold and/or function properly. For example, in some embodiments, a protein encoded by a gene of interest comprises one or more (e.g., 1, 2, 3, 4, 5, or more) disulfide bonds. In some embodiments, a gene of interest encodes an antibody or antigen-binding fragment thereof. In some embodiments, a gene of interest encodes a single-chain variable region (scFv). In some embodiments, a protein comprises trastuzumab (Herceptin®). In some embodiments, a protein comprises an nucleic acid sequence with at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) identity to any one of SEQ ID NO: 21-29. In some embodiments, a protein comprises a nucleic acid sequence of any one of SEQ ID NO: 21-29.

A gene of interest to be evolved may be under the control of a promoter. In some embodiments, the promoter is a constitutive promoter. In some embodiments, the promoter is a conditional promoter, for example an inducible promoter.

In some embodiments, a selection phage (SP) further comprises a periplasmic signal sequence or a fragment thereof. Generally, periplasmic signal sequences are short peptides that enable intracellular trafficking of a protein containing the signal to the periplasmic space of a bacteria cell. In some embodiments, a periplasmic signal sequence comprises between 3 and 25 amino acids. In some embodiments, a periplasmic signal sequences comprises 3, 4, 5, 6, 7, 8, 9, 10, 11, 12, 13, 14, 15, 16, 17, 18, 19, 20, 21, 22, 23, 24, or 25 amino acids. In some embodiments, a periplasmic signal sequence comprises a phosphatase A (PhoA)-derived signal sequence. In some embodiments, a periplasmic signal sequence is connected (e.g., attached or fused to, or expressed as a fusion protein with) a gene product expressed by a gene of interest to be evolved. The periplasmic signal sequence may be positioned N-terminal or C-terminal with respect to the gene product.

Aspects of the disclosure relate to split signal sequences. Splitting the signal sequence directing periplasmic export into two halves, with one half expressed at a controlled level on a host plasmid, allows the extent of export to the periplasm to be defined, thereby providing a way to directly modulate selection stringency in the periplasm. For example, splitting a signal sequence may enable selection for variants that limit aggregation or degradation occurring after intein-mediated splicing, mediate rapid periplasmic export, or facilitate successful periplasmic folding of a gene product expressed by a gene of interest.

In some embodiments, a selection phage (SP) comprises a gene of interest to be evolved fused to a split-intein. An “intein” refers to a protein that is able to self-catalytically excise itself and join the remaining protein fragments (e.g., exteins) by the process of protein splicing. Generally, the self-splicing function of inteins makes them useful tools for engineering trans-spliced recombinant proteins, as described in U.S. Publication No. 2003-0167533, the entire contents of which are incorporated herein by reference. For example, expressing (i) a nucleic acid sequence encoding a N-terminal intein fragment (or portion) operably linked to a nucleic acid encoding a first protein fragment (A) and (ii) a nucleic acid encoding a C-terminal intein fragment (or portion) operably linked to a nucleic acid encoding a second protein fragment (B), in a cell would result, in some embodiments, in trans-splicing of the inteins within the cell to produce a fusion molecule comprising (in the following order) “A-B”.

Inteins are present in both prokaryotic and eukaryotic organisms. In some embodiments, an intein is a bacterial intein, such as a cyanobacterial intein (e.g., intein from Synechocystis or Nostoc). In some embodiments, the intein is a Nostoc punctiforme (Npu) intein, for example, as described in Oeemig et al. (2009) FEBS Lett. 583(9):1451-6.

In some embodiments, a selection phage (SP) described herein further comprises a nucleic acid encoding a split intein portion (e.g., a split intein N-terminal portion or split intein C-terminal portion) operably linked to a nucleic acid encoding a periplasmic signal peptide and the gene of interest. In some embodiments, the split intein portion is a split intein C-terminal portion (e.g., a Npu split intein C-terminal portion). In some embodiments, the split intein C-terminal portion is positioned upstream of (e.g., 5′ relative to) the nucleic acid encoding the periplasmic signal peptide sequence. In some embodiments, the split intein portion is a split intein N-terminal portion (e.g., a Npu split intein N-terminal portion). In some embodiments, the split intein N-terminal portion is positioned downstream of (e.g., 3′ relative to) the nucleic acid encoding the periplasmic signal peptide sequence and the gene of interest.

A selection phage (SP) may further comprise one or more additional molecules (e.g., peptides, proteins, etc.) that interact with, or facilitate interaction with, a periplasmic capture agent. Examples of additional molecules include monobodies and leucine zipper domains. In some embodiments, an additional molecule is SH2, which binds HA4 monobody. In some embodiments, an additional molecule is a GCN4 leucine zipper domain, which dimerizes gene products of interest prior to interaction of the gene products of interest with periplasmic capture agents. Alternative arrangements which could be used include any pairs of small heterodimerizing or homodimerizing proteins with high affinity, such as YibK, Jun/Fos leucine zippers, or monobodies/adnectins coupled with their associated ligands. Examples of monobody/ligand pairs include the monobody ySMB-1 and the SUMO1 protein, or the monobody ysx1 and the maltose-binding protein. In some embodiments, a molecule which binds a monobody comprises a nucleic acid sequence with at least 70% identity to SEQ ID NO: 14. In some embodiments, a molecule which binds a monobody comprises a nucleic acid sequence with at least 80%, at least 90%, at least 95%, or at least 99% identity to SEQ ID NO: 14. In some embodiments, a molecule which binds a monobody comprises or consists of the nucleic acid sequence of SEQ ID NO: 14.

### Accessory Plasmids

Aspects of the disclosure relate to expression constructs (e.g., accessory plasmids (APS), etc.) encoding a fusion protein comprising a DNA binding protein connected to a periplasmic capture agent. “DNA binding protein” generally refers to a protein that has one or more DNA-binding domains and thus has a specific or general affinity for single- or double-stranded DNA. The disclosure is based, in part, on the inclusion of certain DNA binding proteins (or fragments thereof) as mediators which transduce binding of a periplasmic capture agent to a gene product of interest into a signal that results in expression of a gene of interest required for production of infectious phage (e.g., gIII). In some embodiments, a DNA binding protein is a bacterial DNA binding protein or a portion thereof. A “portion” of a DNA binding protein may comprise at least 10%, 20%, 30%, 40%, 50%, 60%, 70%, 80%, 90%, 95%, 99%, or more of the amino acid sequence of a DNA binding protein. In some embodiments, a portion of a DNA binding protein lacks one or more functional domains of the DNA binding protein, for example a periplasmic sensor domain, or a DNA binding domain. In some embodiments, a DNA binding protein or a portion thereof comprises a CadC DNA binding protein or a portion thereof. In some embodiments, a CadC molecule is a variant of a wild-type CadC molecule, for example a CadC protein having the sequence set forth as:

(SEQ ID NO: 33)
MQQPVVRVGEWLVTPSINQISRNGRQLTLEPRLIDLLVFFAQHSGEVLS
RDELIDNVWKRSIVTNHVVTQSISELRKSLKDNDEDSPVYIATVPKRGY
KLMVPVIWYSEEEGEEIMLSSPPPIPEAVPATDSPSHSLNIQNTATPPE
QSPVKSKRFTTFWVWFFFLLSLGICVALVAFSSLDTRLPMSKSRILLNP
RDIDINMVNKSCNSWSSPYQLSYAIGVGDLVATSLNTFSTFMVHDKINY
NIDEPSSSGKTLSIAFVNQRQYRAQQCFMSIKLVDNADGSTMLDKRYVI
TNGNQLAIQNDLLESLSKALNQPWPQRMQETLQKILPHRGALLTNFYQA
HDYLLHGDDKSLNRASELLGEIVQSSPEFTYARAEKALVDIVRHSQHPL
DEKQLAALNTEIDNIVTLPELNNLSIIYQIKAVSALVKGKTDESYQAIN
TGIDLEMSWLNYVLLGKVYEMKGMNREAADAYLTAFNLRPGANTLYWIE
NGIFQTSVPYVVPYLDKFLASE.

In some embodiments, a CadC molecule lacks a periplasmic sensor domain. In some embodiments, the sensor domain comprises the amino acid sequence:

(SEQ ID NO: 35)
SLDTRLPMSKSRILLNPRDIDINMVNKSCNSWSSPYQLSYAIGVGDLVA
TSLNTFSTFMVHDKINYNIDEPSSSGKTLSIAFVNQRQYRAQQCFMSIK
LVDNADGSTMLDKRYVITNGNQLAIQNDLLESLSKALNQPWPQRMQETL
QKILPHRGALLTNFYQAHDYLLHGDDKSLNRASELLGEIVQSSPEFTYA
RAEKALVDIVRHSQHPLDEKQLAALNTEIDNIVTLPELNNLSIIYQIKA
VSALVKGKTDESYQAINTGIDLEMSWLNYVLLGKVYEMKGMNREAADAY
LTAFNLRPGANTLYWIENGIFQTSVPYVVPYLDKFLASE.

A DNA binding protein or portion thereof may be connected to any suitable periplasmic capture agent. In some embodiments, a periplasmic capture agent is selected from an agent (e.g., an antigen) that binds to the gene product expressed by the gene of interest, a monobody, a scFv, or a leucine zipper domain. In some embodiments, the leucine zipper domain comprises a leucine zipper domain of the yeast GCN4 transcription factor. In some embodiments, a GCN4 tag is a mutant GNC4 tag, for example GCN4 7P14P. In some embodiments a mutant GCN4 tag does not dimerize. In some embodiments, a periplasmic capture agent comprises a periplasmic signal peptide sequence or a portion thereof.

In some embodiments, an expression construct described herein comprises a nucleic acid encoding a split intein portion (e.g., a split intein N-terminal portion or split intein C-terminal portion) operably linked to a nucleic acid encoding a gene required for the production of infectious phage particles, such as gIII protein (pIII protein), or a portion (e.g., fragment) thereof. In some embodiments, the split intein portion is a split intein C-terminal portion (e.g., a Npu split intein C-terminal portion). In some embodiments, the split intein C-terminal portion is positioned upstream of (e.g., 5′ relative to) the nucleic acid encoding the gene required for the production of infectious phage particles, or portion thereof. In some embodiments, the split intein portion is a split intein N-terminal portion (e.g., a Npu split intein N-terminal portion). In some embodiments, the split intein N-terminal portion is positioned downstream of (e.g., 3′ relative to) the nucleic acid encoding the gene required for the production of infectious phage particles, or portion thereof.

Aspects of the disclosure relate to expression constructs encoding a pIII protein under the control of a conditional promoter, wherein activation of the conditional promoter is dependent on binding of a first gene product of the gene of interest to the periplasmic capture agent.

In some embodiments, a conditional promoter is activated by binding of a molecule or molecules to at least two proximal DNA binding motifs present within the promoter. When used in the context of binding motifs, ‘proximal’ refers to a distance between two binding motifs which allows the proteins comprising such binding motifs to interact (e.g., dimerize). Proximal binding motifs. In some embodiments, each binding site of a set of “proximal” DNA binding motifs may range from about 2 to about 50 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides in length. In some embodiments, a set of “proximal” DNA binding sites are separated by between 1 and 100 nucleotides. In some embodiments, a set of “proximal” DNA binding sites are separated by between 2 and 15 nucleotides, 5 and 20 nucleotides, 10 and 50 nucleotides, 30 and 70 nucleotides, or 50 and 100 nucleotides.

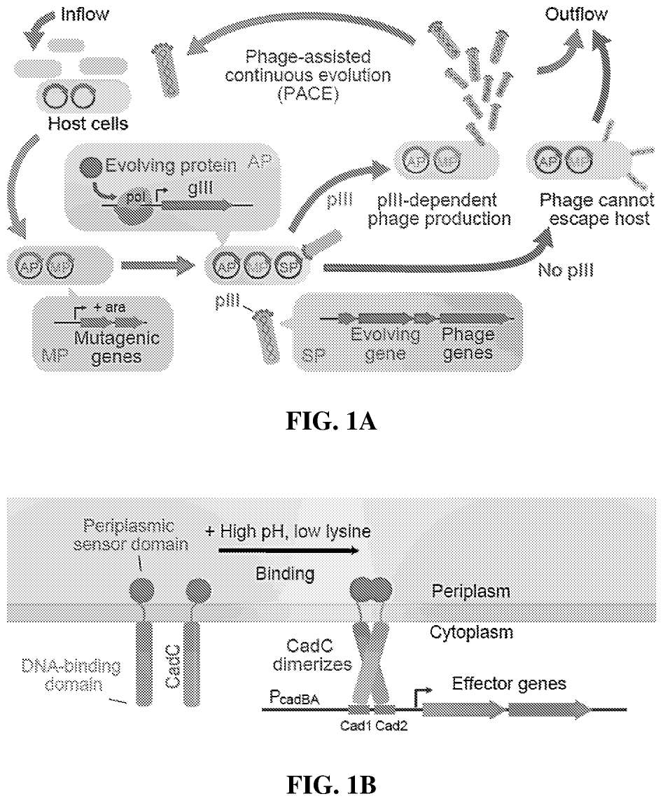

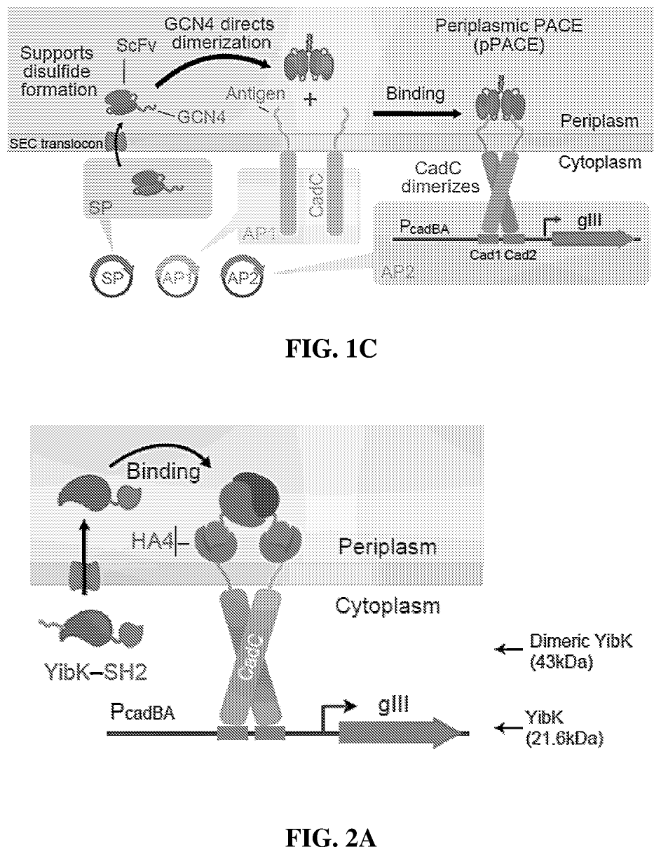

In some embodiments, a promoter comprises one or more E. coli DNA binding protein binding sites. In some embodiments, the E. coli DNA binding protein binding site comprises one or more CadC protein binding sites. CadC is a native E. coli sensor protein and a member of the ToxR-like receptor family. The protein consists of a periplasmic sensor domain, a single transmembrane helix, and a DNA-binding cytoplasmic domain (FIG. 1B). CadC is a transcriptional activator and as a dimer drives activation of promoter P_{cadBA}. Examples of CadC binding sites include but are not limited to Cad1 binding site and Cad2 binding site. In some embodiments, a Cad1 binding site comprises the nucleic acid sequence AAACATTAAATGTTTATCTTTTCATGATATCAACTTGCG (SEQ ID NO: 36). In some embodiments, a Cad 2 binding site comprises the nucleic acid sequence CCTCAAGTTCTCACTTACAGAAACTTTTGT (SEQ ID NO: 37). In some embodiments, a promoter comprises a Cad1 binding motif. In some embodiments, a promoter comprises a Cad2 binding motif. In some embodiments, a promoter comprises a Cad1 and a Cad2 binding motif. In some embodiments, Cad1 and Cad2 DNA motifs comprise the nucleotides between positions −144 to −112 bp and −89 to −59 bp respectively, of promoter P_{cadBA} and are often activated by a dimer of CadC molecules.

By arranging activation of the promoter for a necessary element for phage propagation (e.g., pIII), under the control of a dimer (e.g., CadC, by means of necessary activation at two proximal DNA binding motifs (e.g., Cad1 and Cad2)), tying the activity of bringing two CadC molecules into close vicinity with one another (to promoter dimerization) with the desired activity (e.g., antigen or scFv specificity or binding affinity), transcription and eventual translation of the necessary element for phase propagation (e.g., translation of a gene product required for production of infectious phage, such as pIII) can be controlled by the desired activity.

In some embodiments, a promoter is activated by CadC molecules. In some embodiments, a promoter is activated by a homodimer of CadC molecules. In some embodiments, an expression construct comprises an expression construct which encodes pIII, operably attached to a conditional promoter, wherein the conditional promoter is activated by a homodimer of CadC. In some embodiments, a conditional promoter is P_{cadBA}.

In some embodiments, an expression construct encoding a pIII protein under the control of a conditional promoter further comprises a nucleic acid encoding a split intein portion (e.g., a split intein N-terminal portion or split intein C-terminal portion) linked to a periplasmic signal peptide sequence or a portion thereof. In some embodiments, the split intein portion is a split intein C-terminal portion (e.g., a Npu split intein C-terminal portion). In some embodiments, the split intein C-terminal portion is positioned upstream of (e.g., 5′ relative to) the nucleic acid encoding the gene required for the production of infectious phage particles, or portion thereof. In some embodiments, the split intein portion is a split intein N-terminal portion (e.g., a Npu split intein N-terminal portion). In some embodiments, the split intein N-terminal portion is positioned downstream of (e.g., 3′ relative to) the nucleic acid encoding the gene required for the production of infectious phage particles, or portion thereof.

In some aspects, the disclosure relates to expression vectors (e.g., plasmids) comprising a gene of interest to be evolved fused to a sequence encoding a therapeutic protein. In some embodiments, a protein is a single chain variable fragment (scFv). ScFvs comprise only the heavy and light chain variable antigen binding regions (VH and VL respectively) tethered by a flexible synthetic linker. ScFvs are small in size (˜30 kDa), can be produced in E. coli, exhibit improved tissue penetration, and can be readily conjugated to drug molecules, effector proteins and chimeric antigen receptors, making them prime candidate molecules for directed evolution approaches. Heterologous expression of scFvs in E. coli typically involves tagging them for export into the periplasm using an N-terminal signal sequence peptide. In some embodiments, the plasmid is a selection plasmid (e.g., selection phagemid). In some embodiments, the expression construct comprises a nucleic acid encoding the gene of interest is contiguous (e.g., operably linked) to the nucleic acid sequence encoding the protein of interest (e.g., first gene product). In some embodiments, the 3′-end of the nucleic acid encoding the gene of interest is contiguous (e.g., operably linked) to the 5′-end of the nucleic acid encoding the protein of interest (e.g., first gene product). In some embodiments, a nucleic acid comprises a first expression construct. In some embodiments, a first expression construct is under the control of a promoter. In some embodiments, a promoter is a conditional promoter. In some embodiments, a conditional promoter comprises a P_{BAD} promoter. In some embodiments, a conditional promoter is a P_{T7LaC}, P_{Rhanmose} and P_{yieW} promoter.

In some embodiments, the nucleic acid encoding a gene required for the production of infectious phage particles, such as gIII protein (pIII protein), is truncated (e.g., missing one or more nucleic acid bases relative to a full-length gene encoding pIII protein), but is functional. In some embodiments, the nucleic acid is 1, 2, 3, 4, 5, 6, 7, 8, 9, 10, or more nucleic acid bases shorter than a full-length gene encoding pIII protein. It should be appreciated that the nucleic acid encoding truncated pIII protein may be truncated at either the 5′-end or the 3′-end.

The first expression construct and the second expression construct can be located on the same vector (e.g., plasmid) or on separate vectors (e.g., different plasmids). In some embodiments, the vector is an accessory plasmid (AP). In some embodiments, a bacterial 2-hybrid system comprises a third expression construct comprising a nucleic acid encoding a gene of interest to be evolved (e.g., a HA4 monobody).

Additional selection systems may be used in conjunction with methods described herein. A selection system can be a positive selection system, a negative selection system or a combination of one or more positive selection systems (e.g., 1, 2, 3, 4, 5, or more positive selection systems) and one or more negative selection systems (e.g., 1, 2, 3, 4, 5, or more negative selection systems). In some embodiments, a positive selection system links production (e.g., translation and/or function) of an evolved protein having a desired physiochemical characteristic (e.g., binding affinity, solubility, stability, etc.) and/or a desired function to expression of a gene required for production of infectious phage particles. In some embodiments, a negative selection system links production (e.g., translation and/or function) of an evolved protein having an undesired physiochemical characteristic (e.g., reduced solubility, reduced stability, etc.) and/or an undesired function to expression of a gene that prevents production of infectious phage particles (e.g., dominant negative pIII protein, such as pIII-neg). In the context of PACE, suitable negative selection strategies and reagents are described herein and in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Ser. No. 13/922,812, filed Jun. 20, 2013; and U.S. Application, U.S. Ser. No. 62/067,194, filed Oct. 22, 2014, the entire contents of each of which are incorporated herein by reference.

### Methods

In some aspects, the disclosure provides methods for directed evolution using one or more of the expression constructs described herein. In some embodiments, the method comprises (a) contacting a population of host cells comprising an expression construct or plasmid as provided herein with a population of phage vectors comprising a gene to be evolved and deficient in at least one gene for the generation of infectious phage particles, wherein (1) the host cells are amenable to transfer of the vector; (2) the vector allows for expression of the gene to be evolved in the host cell, can be replicated by the host cell, and the replicated vector can transfer into a second host cell; (3) the host cell expresses a gene product encoded by the at least one gene for the generation of infectious phage particles of (a) in response to a particular physiochemical characteristic (e.g., solubility, stability, etc.) and/or activity of the gene to be evolved in the periplasm of the host cell, and the level of gene product expression depends on the physiochemical characteristic and/or activity of the gene to be evolved in the periplasm of the host cell; (b) incubating the population of host cells (e.g., a plurality of host cells) under conditions allowing for selection of the gene to be evolved based upon the physiochemical characteristic and/or activity of the gene to be evolved and the transfer of the vector comprising the gene to be evolved from host cell to host cell, wherein host cells are removed from the host cell population, and the population of host cells is replenished with fresh host cells that comprise the expression construct but do not harbor the vector; and (c) isolating a replicated vector from the host cell population in (b), wherein the replicated vector comprises a mutated version of the gene to be evolved (e.g., an evolved protein).

In some embodiments, the expression construct comprises an inducible promoter, wherein the incubating of (b) comprises culturing the population of host cells under conditions suitable to induce expression from the inducible promoter. In some embodiments, the inducible promoter is an arabinose-inducible promoter, wherein the incubating of (b) comprises contacting the host cell with an amount of arabinose sufficient to increase expression of the arabinose-inducible promoter by at least 10-fold, at least 50-fold, at least 100-fold, at least 500-fold, at least 1000-fold, at least 5000-fold, at least 10000-fold, at least 50000-fold, at least 100000-fold, at least 500000-fold, or at least 1000000-fold as compared to basal expression in the absence of arabinose. In some embodiments, a promoter is an arabinose inducible promoter.

In some embodiments, the vector is a viral vector. In some embodiments, the viral vector is a phage. In some embodiments, the phage is a filamentous phage. In some embodiments, the phage is an M13 phage.

In some embodiments, the host cells comprise an accessory plasmid. In some embodiments, the accessory plasmid comprises an expression construct encoding the pIII protein under the control of a promoter that is activated by a gene product encoded by the gene to be evolved. In some embodiments, the host cells comprise the accessory plasmid and together, the helper phage and the accessory plasmid comprise all genes required for the generation of an infectious phage. In some embodiments, the method further comprises a negative selection for undesired activity of the gene to be evolved. In some embodiments, the host cells comprise an expression construct encoding a dominant-negative pIII protein (pIII-neg). In some embodiments, expression of the pIII-neg protein is driven by a promoter the activity of which depends on an undesired function of the gene to be evolved.

In some embodiments, step (b) comprises incubating the population of host cells for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive life cycles of the viral vector or phage. In some embodiments, the host cells are E. coli cells.

In some embodiments, the host cells are incubated in suspension culture. In some embodiments, the population of host cells is continuously replenished with fresh host cells that do not comprise the vector. In some embodiments, fresh cells are being replenished and cells are being removed from the cell population at a rate resulting in a substantially constant number of cells in the cell population. In some embodiments, fresh cells are being replenished and cells are being removed from the cell population at a rate resulting in a substantially constant vector population. In some embodiments, fresh cells are being replenished and cells are being removed from the cell population at a rate resulting in a substantially constant vector, viral, or phage load. In some embodiments, the rate of fresh cell replenishment and/or the rate of cell removal is adjusted based on quantifying the cells in the cell population. In some embodiments, the rate of fresh cell replenishment and/or the rate of cell removal is adjusted based on quantifying the frequency of host cells harboring the vector and/or of host cells not harboring the vector in the cell population. In some embodiments, the quantifying is by measuring the turbidity of the host cell culture, measuring the host cell density, measuring the wet weight of host cells per culture volume, or by measuring light extinction of the host cell culture.

In some embodiments, the vector or phage encoding the gene to be evolved is a filamentous phage, for example, an M13 phage, such as an M13 selection phage as described in more detail elsewhere herein. In some embodiments, the host cells are cells amenable to infection by the filamentous phage, e.g., by M13 phage, such as, for example, E. coli cells. In some such embodiments, the gene required for the production of infectious viral particles is the M13 gene III (gIII) encoding the M13 protein III (pIII).

Typically, the vector/host cell combination is chosen in which the life cycle of the vector is significantly shorter than the average time between cell divisions of the host cell. Average cell division times and vector life cycle times are well known in the art for many cell types and vectors, allowing those of skill in the art to ascertain such host cell/vector combinations. In certain embodiments, host cells are being removed from the population of host cells in which the vector replicates at a rate that results in the average time of a host cell remaining in the host cell population before being removed to be shorter than the average time between cell divisions of the host cells, but to be longer than the average life cycle of the viral vector employed. The result of this is that the host cells, on average, do not have sufficient time to proliferate during their time in the host cell population while the viral vectors do have sufficient time to infect a host cell, replicate in the host cell, and generate new viral particles during the time a host cell remains in the cell population. This assures that the only replicating nucleic acid in the host cell population is the vector encoding the gene to be evolved, and that the host cell genome, the accessory plasmid, or any other nucleic acid constructs cannot acquire mutations allowing for escape from the selective pressure imposed.

For example, in some embodiments, the average time a host cell remains in the host cell population is about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes.

In some embodiments, the average time a host cell remains in the host cell population depends on how fast the host cells divide and how long infection (or conjugation) requires. In general, the flow rate should be faster than the average time required for cell division, but slow enough to allow viral (or conjugative) propagation. The former will vary, for example, with the media type, and can be delayed by adding cell division inhibitor antibiotics (FtsZ inhibitors in E. coli, etc.). Since the limiting step in continuous evolution is production of the protein required for gene transfer from cell to cell, the flow rate at which the vector washes out will depend on the current activity of the gene(s) of interest. In some embodiments, titrable production of the protein required for the generation of infectious particles, as described herein, can mitigate this problem. In some embodiments, an indicator of phage infection allows computer-controlled optimization of the flow rate for the current activity level in real-time.

In some embodiments, a PACE experiment according to methods provided herein is run for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, the host cells are contacted with the vector and/or incubated in suspension culture. For example, in some embodiments, bacterial cells are incubated in suspension culture in liquid culture media. Suitable culture media for bacterial suspension culture will be apparent to those of skill in the art, and the invention is not limited in this regard. See, for example, Molecular Cloning: A Laboratory Manual, 2nd Ed., ed. by Sambrook, Fritsch, and Maniatis (Cold Spring Harbor Laboratory Press: 1989); Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable culture media for bacterial host cell culture).

Suspension culture typically requires the culture media to be agitated, either continuously or intermittently. This is achieved, in some embodiments, by agitating or stirring the vessel comprising the host cell population. In some embodiments, the outflow of host cells and the inflow of fresh host cells is sufficient to maintain the host cells in suspension. This in particular, if the flow rate of cells into and/or out of the lagoon is high.

In some embodiments, the flow of cells through the lagoon is regulated to result in an essentially constant number of host cells within the lagoon. In some embodiments, the flow of cells through the lagoon is regulated to result in an essentially constant number of fresh host cells within the lagoon. Typically, the lagoon will hold host cells in liquid media, for example, cells in suspension in a culture media. However, lagoons in which adherent host cells are cultured on a solid support, such as on beads, membranes, or appropriate cell culture surfaces are also envisioned. The lagoon may comprise additional features, such as a stirrer or agitator for stirring or agitating the culture media, a cell densitometer for measuring cell density in the lagoon, one or more pumps for pumping fresh host cells into the culture vessel and/or for removing host cells from the culture vessel, a thermometer and/or thermocontroller for adjusting the culture temperature, as well as sensors for measuring pH, osmolarity, oxygenation, and other parameters of the culture media. The lagoon may also comprise an inflow connected to a holding vessel comprising a mutagen or a transcriptional inducer of a conditional gene expression system, such as the arabinose-inducible expression system of the mutagenesis plasmid described in more detail elsewhere herein.

In some embodiments, the host cell population is continuously replenished with fresh, uninfected host cells. In some embodiments, this is accomplished by a steady stream of fresh host cells into the population of host cells. In other embodiments, however, the inflow of fresh host cells into the lagoon is semi-continuous or intermittent (e.g., batch-fed). In some embodiments, the rate of fresh host cell inflow into the cell population is such that the rate of removal of cells from the host cell population is compensated. In some embodiments, the result of this cell flow compensation is that the number of cells in the cell population is substantially constant over the time of the continuous evolution procedure. In some embodiments, the portion of fresh, uninfected cells in the cell population is substantially constant over the time of the continuous evolution procedure. For example, in some embodiments, about 10%, about 15%, about 20%, about 25%, about 30%, about 40%, about 50%, about 60%, about 70%, about 75%, about 80%, or about 90% of the cells in the host cell population are not infected by virus. In general, the faster the flow rate of host cells is, the smaller the portion of cells in the host cell population that are infected will be. However, faster flow rates allow for more transfer cycles, e.g., viral life cycles, and, thus, for more generations of evolved vectors in a given period of time, while slower flow rates result in a larger portion of infected host cells in the host cell population and therefore a larger library size at the cost of slower evolution. In some embodiments, the range of effective flow rates is invariably bounded by the cell division time on the slow end and vector washout on the high end In some embodiments, the viral load, for example, as measured in infectious viral particles per volume of cell culture media is substantially constant over the time of the continuous evolution procedure.

The pPACE methods provided herein are typically carried out in a lagoon. Suitable lagoons and other laboratory equipment for carrying out PACE methods as provided herein have been described in detail elsewhere. See, for example, International PCT Application, PCT/US2011/066747, published as WO2012/088381 on Jun. 28, 2012, the entire contents of which are incorporated herein by reference. In some embodiments, the lagoon comprises a cell culture vessel comprising an actively replicating population of vectors, for example, phage vectors comprising a gene of interest, and a population of host cells, for example, bacterial host cells. In some embodiments, the lagoon comprises an inflow for the introduction of fresh host cells into the lagoon and an outflow for the removal of host cells from the lagoon. In some embodiments, the inflow is connected to a turbidostat comprising a culture of fresh host cells. In some embodiments, the outflow is connected to a waste vessel, or a sink. In some embodiments, the lagoon further comprises an inflow for the introduction of a mutagen into the lagoon. In some embodiments that inflow is connected to a vessel holding a solution of the mutagen. In some embodiments, the lagoon comprises an inflow for the introduction of an inducer of gene expression into the lagoon, for example, of an inducer activating an inducible promoter within the host cells that drives expression of a gene promoting mutagenesis (e.g., as part of a mutagenesis plasmid), as described in more detail elsewhere herein. In some embodiments, that inflow is connected to a vessel comprising a solution of the inducer, for example, a solution of arabinose.

In some embodiments, the lagoon comprises a controller for regulation of the inflow and outflow rates of the host cells, the inflow of the mutagen, and/or the inflow of the inducer. In some embodiments, a visual indicator of phage presence, for example, a fluorescent marker, is tracked and used to govern the flow rate, keeping the total infected population constant. In some embodiments, the visual marker is a fluorescent protein encoded by the phage genome, or an enzyme encoded by the phage genome that, once expressed in the host cells, results in a visually detectable change in the host cells. In some embodiments, the visual tracking of infected cells is used to adjust a flow rate to keep the system flowing as fast as possible without risk of vector washout.

In some embodiments, the controller regulates the rate of inflow of fresh host cells into the lagoon to be substantially the same (volume/volume) as the rate of outflow from the lagoon. In some embodiments, the rate of inflow of fresh host cells into and/or the rate of outflow of host cells from the lagoon is regulated to be substantially constant over the time of a continuous evolution experiment. In some embodiments, the rate of inflow and/or the rate of outflow is from about 0.1 lagoon volumes per hour to about 25 lagoon volumes per hour. In some embodiments, the rate of inflow and/or the rate of outflow is approximately 0.1 lagoon volumes per hour (lv/h), approximately 0.2 lv/h, approximately 0.25 lv/h, approximately 0.3 Iv/h, approximately 0.4 lv/h, approximately 0.5 lv/h, approximately 0.6 lv/h, approximately 0.7 lv/h, approximately 0.75 lv/h, approximately 0.8 lv/h, approximately 0.9 lv/h, approximately 1 lv/h, approximately 2 lv/h, approximately 2.5 lv/h, approximately 3 lv/h, approximately 4 lv/h, approximately 5 lv/h, approximately 7.5 lv/h, approximately 10 lv/h, or more than 10 lv/h.

In some embodiments, the inflow and outflow rates are controlled based on a quantitative assessment of the population of host cells in the lagoon, for example, by measuring the cell number, cell density, wet biomass weight per volume, turbidity, or cell growth rate. In some embodiments, the lagoon inflow and/or outflow rate is controlled to maintain a host cell density of from about 10² cells/ml to about 10¹² cells/ml in the lagoon. In some embodiments, the inflow and/or outflow rate is controlled to maintain a host cell density of about 10² cells/ml, about 10³ cells/ml, about 10⁴ cells/ml, about 10⁵ cells/ml, about 5×10⁵ cells/ml, about 10⁶ cells/ml, about 5×10⁶ cells/ml, about 10⁷ cells/ml, about 5×10⁷ cells/ml, about 10⁸ cells/ml, about 5×10⁸ cells/ml, about 10⁹ cells/ml, about 5×10⁹ cells/ml, about 10¹⁰ cells/ml, about 5×10¹⁰ cells/ml, or more than 5×10¹⁰ cells/ml, in the lagoon. In some embodiments, the density of fresh host cells in the turbidostat and the density of host cells in the lagoon are substantially identical.

In some embodiments, the lagoon inflow and outflow rates are controlled to maintain a substantially constant number of host cells in the lagoon. In some embodiments, the inflow and outflow rates are controlled to maintain a substantially constant frequency of fresh host cells in the lagoon. In some embodiments, the population of host cells is continuously replenished with fresh host cells that are not infected by the phage. In some embodiments, the replenishment is semi-continuous or by batch-feeding fresh cells into the cell population.

In some embodiments, the lagoon volume is from approximately 1 ml to approximately 100 l, for example, the lagoon volume is approximately 1 ml, approximately 10 ml, approximately 50 ml, approximately 100 ml, approximately 200 ml, approximately 250 ml, approximately 500 ml, approximately 750 ml, approximately 1 l, approximately 2 l, approximately 2.5 l, approximately 3 l, approximately 4 l, approximately 5 l, approximately 10 l, approximately 20 l, approximately 50 l, approximately 75 l, approximately 100 l, approximately 1 ml-10 ml, approximately 10 ml-50 ml, approximately 50 ml-100 ml, approximately 100 ml-250 ml, approximately 250 ml-500 ml, approximately 500 ml-1 l, approximately 1 l-2 l, approximately 2 l-5 l, approximately 5 l-10 l, approximately 10 l-50 l, approximately 50 l-100 l, or more than 100 l.

In some embodiments, the lagoon and/or the turbidostat further comprises a heater and a thermostat controlling the temperature. In some embodiments, the temperature in the lagoon and/or the turbidostat is controlled to be from about 4° C. to about 55° C., preferably from about 25° C. to about 39° C., for example, about 37° C.

In some embodiments, the inflow rate and/or the outflow rate is controlled to allow for the incubation and replenishment of the population of host cells for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive vector or phage life cycles. In some embodiments, the time sufficient for one phage life cycle is about 10, 15, 20, 25, or 30 minutes.

Therefore, in some embodiments, the time of the entire evolution procedure is about 12 hours, about 18 hours, about 24 hours, about 36 hours, about 48 hours, about 50 hours, about 3 days, about 4 days, about 5 days, about 6 days, about 7 days, about 10 days, about two weeks, about 3 weeks, about 4 weeks, or about 5 weeks.

In some embodiments, a PACE method as provided herein is performed in a suitable apparatus as described herein. For example, in some embodiments, the apparatus comprises a lagoon that is connected to a turbidostat comprising a host cell as described herein. In some embodiments, the host cell is an E. coli host cell. In some embodiments, the host cell comprises a mutagenesis expression construct as provided herein, an accessory plasmid as described herein, and, optionally, a helper plasmid as described herein, or any combination thereof. In some embodiments, the lagoon further comprises a selection phage as described herein, for example, a selection phage encoding a gene of interest. In some embodiments, the lagoon is connected to a vessel comprising an inducer for a mutagenesis plasmid, for example, arabinose. In some embodiments, the host cells are E. coli cells comprising the F′ plasmid, for example, cells of the genotype F′proA⁺B⁺Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ⁻.

For example, in some embodiments, a PACE method as provided herein is carried out in an apparatus comprising a lagoon of about 100 ml, or about 1 l volume, wherein the lagoon is connected to a turbidostat of about 0.51, 11, or 3 l volume, and to a vessel comprising an inducer for a mutagenesis plasmid, for example, arabinose, wherein the lagoon and the turbidostat comprise a suspension culture of E. coli cells at a concentration of about 5×10⁸ cells/ml. In some embodiments, the flow of cells through the lagoon is regulated to about 3 lagoon volumes per hour. In some embodiments, cells are removed from the lagoon by continuous pumping, for example, by using a waste needle set at a height of the lagoon vessel that corresponds to a desired volume of fluid (e.g., about 100 ml, in the lagoon. In some embodiments, the host cells are E. coli cells comprising any of the nucleic acids of the present disclosure.

### Host Cells

Some aspects of this invention relate to host cells for continuous evolution processes as described herein. In some embodiments, a host cell is provided that comprises a periplasmic space as defined herein above. In some embodiments, a host cell is an E. coli cell. In some embodiments, a host cell is provided that comprises a mutagenesis expression construct as provided herein. In some embodiments, the host cell further comprises additional plasmids or constructs for carrying out a PACE process, e.g., a selection system comprising at least one viral gene encoding a protein required for the generation of infectious viral particles under the control of a conditional promoter the activity of which depends on a desired function of a gene to be evolved. For example, some embodiments provide host cells for phage-assisted continuous evolution processes, wherein the host cell comprises an accessory plasmid comprising a gene required for the generation of infectious phage particles, for example, M13 gIII, under the control of a conditional promoter, as described herein. In some embodiments, the host cell further provides any phage functions that are not contained in the selection phage, e.g., in the form of a helper phage. In some embodiments, the host cell provided further comprises one or more expression constructs (e.g., 1, 2, 3, 4, 5, or more accessory plasmids) comprising a selection system as described herein.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect.

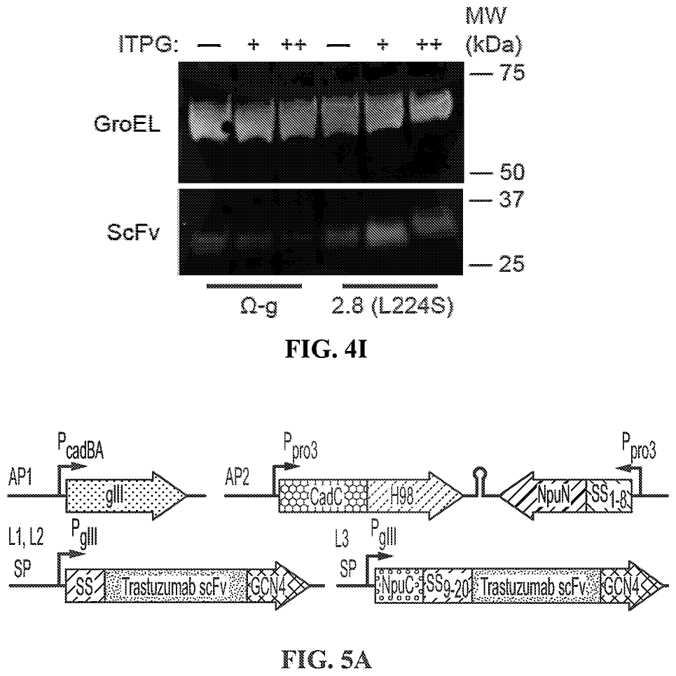

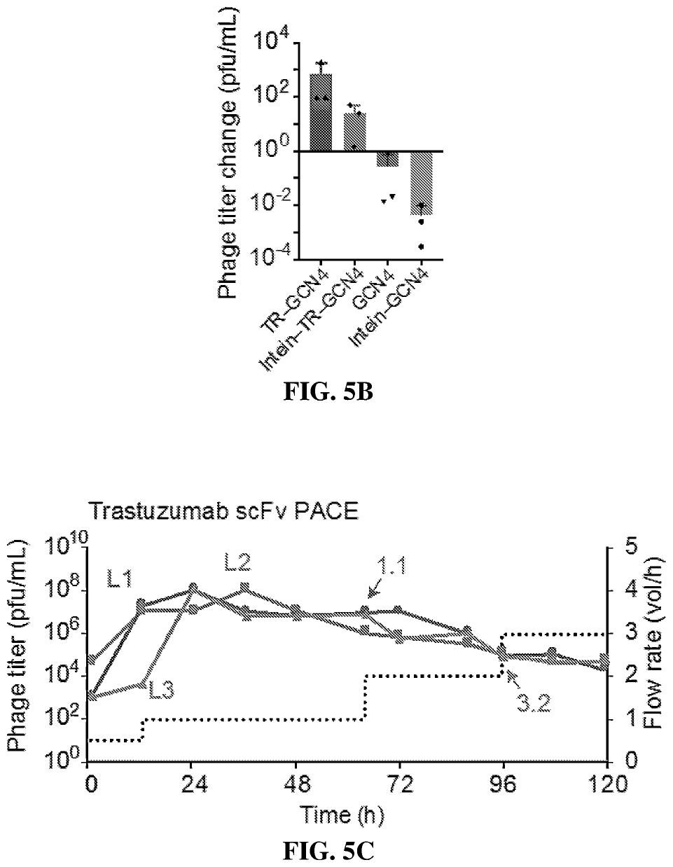

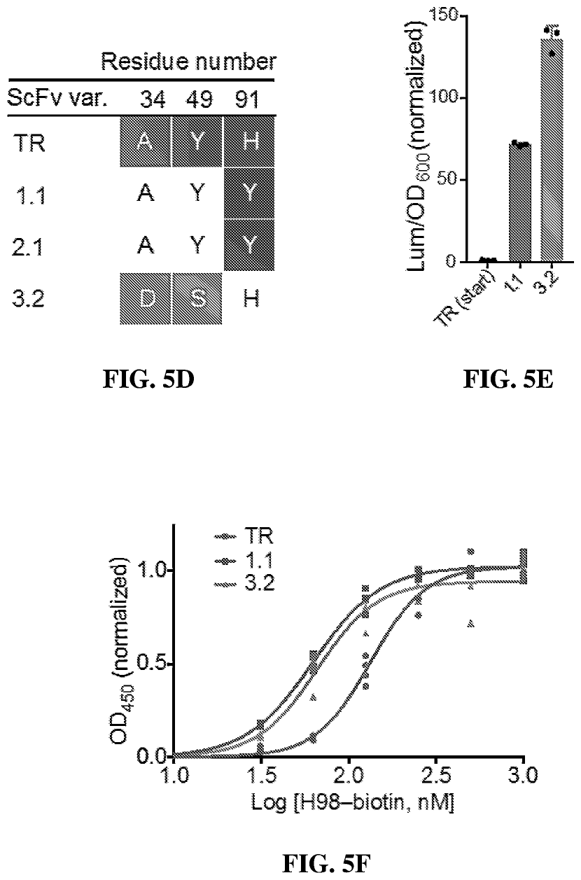

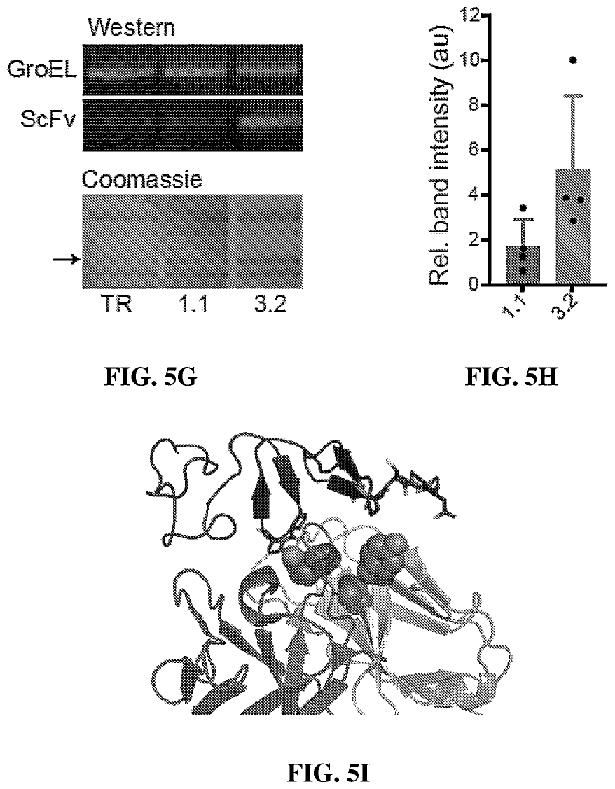

In some pPACE embodiments, for example, in embodiments employing an M13 selection phage, the host cells are E. coli cells expressing the trastuzumab (Herceptin), fragment thereof, or functional equivalent thereof (e.g., scFv). Trastuzumab targets the oncogenic receptor tyrosine kinase Her2 and is a successful first-line treatment for Her2⁺ breast cancers. In some embodiments, a host cell expresses a scFv of trastuzumab comprising any of the mutations found in FIG. 5D (e.g., A34D, Y49S, H91Y, or combination thereof).

### PACE Apparatus

In some embodiments, a pPACE apparatus is provided, comprising a lagoon that is connected to a turbidostat comprising a host cell as described herein. In some embodiments, the host cell is an E. coli host cell. In some embodiments, the host cell comprises one or more accessory plasmids as described herein (e.g., 1, 2, 3, 4, 5, or more accessory plasmids), and optionally, a helper plasmid as described herein or a mutagenesis plasmid as described herein, or any combination thereof. In some embodiments, the lagoon further comprises a selection phage as described herein, for example, a selection phage encoding a gene of interest. In some embodiments, the lagoon is connected to a vessel comprising an inducer for a mutagenesis plasmid, for example, arabinose. In some embodiments, the host cells are E. coli cells comprising the F′ plasmid, for example, cells of the genotype F′proA⁺B⁺Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ.

### EXAMPLES

### Example 1

Antibodies and their engineered derivatives are important treatments for various inflammatory, autoimmune, and infectious diseases, as well as many cancers, including HER2-positive breast cancer, non-Hodgkin's lymphoma, and melanoma. Monoclonal antibodies (mAbs) and their derivatives now represent the largest class of therapeutic protein drugs, with 82 therapeutic antibodies currently approved by the FDA and hundreds in clinical trials.

Antibody-based therapies are limited by high development and production costs. Directed evolution has the potential to decrease cost and accelerate the development of novel and potent antibodies. While multiple selection systems have been shown to evolve new antibody-antigen interactions in E. coli including phage display, APEx, FLI-TRAP, cyclonal, BAD, inner-membrane display, and AHEAD, many of these techniques require researcher intervention to carry out time-intensive steps of each round of evolution. Continuous evolution platforms, in which all stages of the evolutionary cycle are carried out by automated or in vivo processes without the need for researcher invention, have the potential to substantially streamline antibody development as well as the development of other proteins.

Phage-assisted continuous evolution (PACE) is a rapid directed evolution system capable of evolving proteins over days or weeks, with minimal required human intervention during evolution. In PACE, an evolving protein of interest is encoded in place of gene III (gIII) in the genome of M13 bacteriophage (FIG. 1A). An accessory plasmid (AP) within a host E. coli cell expresses gIII under the control of a transcriptional circuit that is activated in response to the desired function of the evolving protein. As phage depend on pIII, the protein product of gIII, to efficiently infect host cells, PACE links the desired property of an evolving protein with the ability of the phage that encodes it to replicate. Phage continuously propagate in a fixed-volume vessel (“lagoon”) that is constantly diluted with a constant inflow of new host E. coli cells from a population maintained in a chemostat. Inflow of new host cells is balanced by outflow to waste, such that phage that fail to propagate are quickly washed out of the lagoon by inflowing cells. Selection pressure is controlled by moderating the flow rate through the lagoon, and through modification of the gene circuit governing gIII expression on the AP. Mutation of the evolving gene occurs simultaneous with selection during PACE by using inducible mutagenesis plasmids (MPs) that elevate the error rate of phage DNA replication when induced. One complete generation of evolution (mutation, Darwinian selection, and replication) occurs with each phage reproductive cycle, which takes place every ˜10 minutes to 1 hour.

PACE has been used to evolve diverse classes of proteins with new activities and specificities, including polymerases, proteases, tRNA synthetases, agricultural toxins, TALENs, Cas9 variants, dehydrogenases, deaminases, antibody fragments, cytosine base editors, and adenine base editors.

However, continuous in vivo evolution platforms, including PACE, have thus far been limited to evolving proteins in the cytoplasm of the host cell. Confining selection to the cytoplasm provides a convenient way to maintain the linkage between genotype and phenotype, and also facilitates mutagenesis, transcription, and translation, streamlining the Darwinian selection process. In both prokaryotes and eukaryotes, however, the cytoplasm is a chemically reducing environment and does not support the formation of disulfide linkages between cysteine residues. Disulfide bonds are crucial determinants of stability and proper folding for many proteins, including antibodies and antibody fragments. The loss of a single disulfide bond can dramatically reduce protein stability and abrogate protein function. Loss of stabilizing disulfide bonds often leads to aggregation during cytoplasmic expression, making disulfide-enriched proteins a challenging class of proteins to evolve by currently available continuous directed evolution techniques.

Disulfide bond formation can be supported in the cytosol through expression of a thiol oxidase and a disulfide isomerase in the cytoplasmic space, but introducing non-native oxidative chemistry into the bacterial cytoplasm increases cellular stress and can lead to membrane impairment and aggregation, a hurdle for the continuous-flow and liquid-handling devices used in continuous directed evolution techniques. Alternatively, directed evolution can be applied to an evolving protein to compensate for loss of disulfides and render a protein biologically active in the reducing cytoplasm, but this process adds complexity and steps which are not ultimately necessary to proteins intended for use outside the cell. Compensatory stabilizing mutations may also result in trade-off costs to target affinity or other biological functions, limiting the scope and relevance of the resulting proteins for use outside of cells. Finally, binding affinity evolutions in the reducing cytoplasm are limited to interactions in which the target protein being bound does not itself rely on disulfides to fold, excluding disulfide-containing extracellular antigens of therapeutic interest. It is thus more biologically relevant to evolve disulfide-containing proteins in oxidizing environments than in reducing environments if the evolving protein is intended for extracellular use.

The bacterial periplasm is an oxidizing environment that supports the formation of disulfides in proteins, such as antibodies and their derivatives. Expression of evolving proteins in the periplasm permits disulfide bond formation while retaining the evolving protein within the bacterial host cell. Linking a protein's desired activity in the periplasm to phage propagation could enable the continuous evolution of proteins that require a non-reducing environment to function.

In this study, a PACE system was developed for the continuous evolution of proteins in the periplasmic space. This platform supports the formation of disulfide bonds in the evolving protein of interest and represents, the first application of PACE to interactions occurring in a cellular compartment other than the cytoplasm and the first continuous in vivo evolution of proteins under oxidizing conditions. Periplasmic PACE (pPACE) can be tuned to select for enhanced soluble expression in addition to enhanced binding activity.

pPACE was validated by using it to restore binding in the homodimeric protein YibK and in the Ω-graft scFv. pPACE was then applied to evolve a minimized form of the antibody drug trastuzumab (Herceptin), achieving up to 2.5-fold improved binding of a Her2-mimetic peptide and 6-fold increased soluble expression, without any loss of native Her2 affinity. Together, these results establish pPACE as a technology that substantially expands the scope of continuous protein evolution to include proteins that require a non-reducing environment to fold and/or function.

### Engineered CadC Activates Transcription Upon Periplasmic Binding

A successful protein-protein interaction selection system that operates in the periplasmic space must convert a binding event in the periplasm into a transcriptional activation event in the cytoplasm. Transmembrane signaling proteins were examined that physically link protein-protein binding in the periplasm with transcription in the cytoplasm. CadC is a native E. coli sensor protein and a member of the ToxR-like receptor family. CadC consists of a periplasmic sensor domain, a single transmembrane helix, and a DNA-binding cytoplasmic domain (FIG. 1B). Under conditions of acidic pH and high lysine concentrations, the periplasmic sensor domains from two CadC molecules homodimerize, bringing together the transmembrane domains and cytoplasmic DNA-binding domains. DNA-binding domain juxtaposition generates two cooperative DNA-binding sites, which then bind two proximal DNA motifs, Cad1 and Cad2, on the CadBA promoter (P_{cadBA}) to initiate gene transcription. Replacement of the periplasmic sensor domain with a dimerizing protein leads to constitutive activation of P_{cadBA}⁵⁷. CadC thus converts a binding event in the periplasm mediated by a modular sensor domain into a cytoplasmic transcriptional activation event.

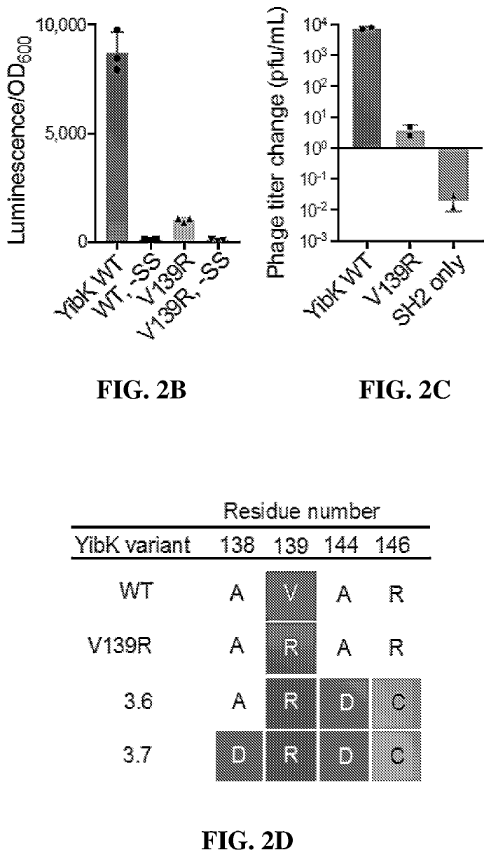

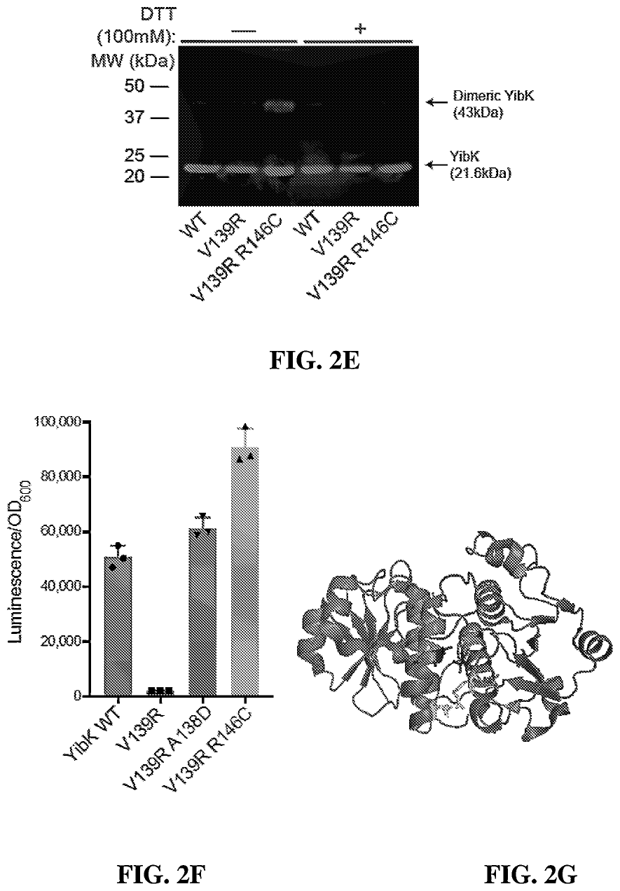

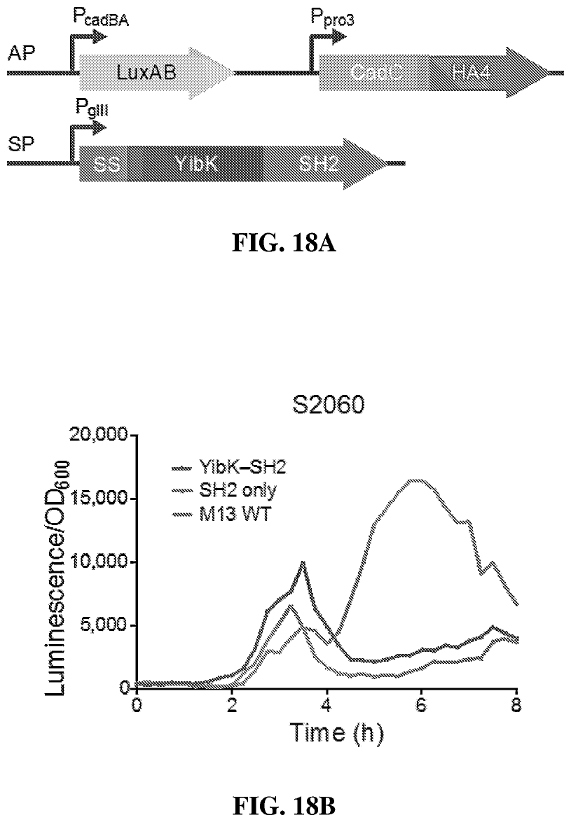

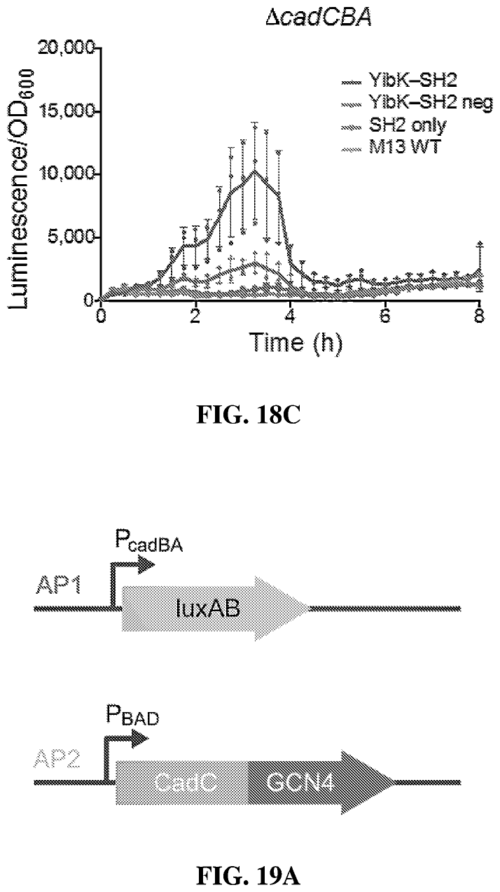

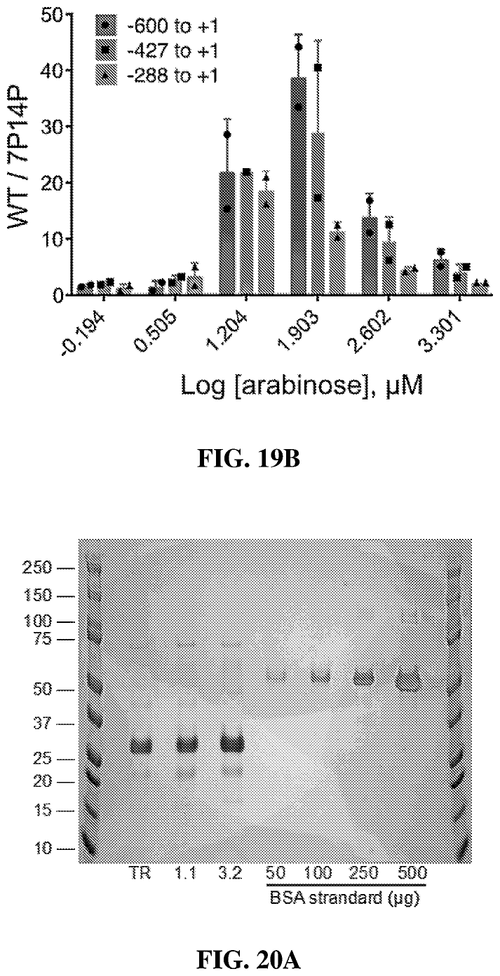

It was reasoned that CadC could form the basis of a PACE selection for protein-protein binding in the periplasmic space (FIG. 1C). First, P_{cadBA} was optimized (FIG. 19), and the host genomic cadCBA operon was deleted to minimize background transcriptional activation (FIGS. 18A-18C). To validate that target protein binding could trigger transcription at P_{cadBA}, CadC was expressed with its sensory domain replaced by the HA4 monobody, a high-affinity antibody mimetic that binds the SH2 domain of ABL1 kinase. YibK was then expressed, a homodimeric knottin protein, fused to the SH2 binding target of HA4. This construct was directed to the periplasm by an N-terminal signal sequence (SS) peptide derived from alkaline phosphatase A (PhoA), a periplasmic E. coli protein. YibK homodimerization should trigger dimerization of the CadC-HA4 fusion via binding of HA4 to the SH2 domain fused to YibK, resulting in activation of P_{cadBA}. Indeed, it was observed that expression of periplasmic YibK-SH2 directed P_{cadBA} transcriptional activation 66-fold over expression of cytoplasmic YibK-SH2 as measured by production of the luminescence reporter LuxAB (FIG. 2B).

The point mutation V139R blocks YibK dimerization by disrupting the hydrophobic interaction surface between YibK monomers and preventing a final folding transition to the native YibK structure. The Kp values for dimerization of wild-type YibK and V139R YibK are <1 pM and 360 μM, respectively. Introduction of V139R resulted in >8-fold loss of P_{cadBA}-directed LuxAB expression (FIG. 2B), establishing that protein-protein affinity determines the degree of transcriptional activation at P_{cadBA}.

To link binding in the periplasm to phage propagation, gIII expression was placed under the control of P_{cadBA}. Phage encoding periplasm-directed YibK-SH2 was challenged in place of gIII to propagate overnight in culture on host cells expressing CadC-HA4 and P_{cadBA}-driven gIII. Phage encoding wild-type YibK propagated more than three orders of magnitude more efficiently in this periplasmic PACE system than V139R YibK phage, demonstrating that pPACE links target protein binding in the periplasm to phage propagation through P_{cadBA} activation and production of pIII (FIG. 2C).

### Periplasmic Phage-Assisted Evolution of YibK

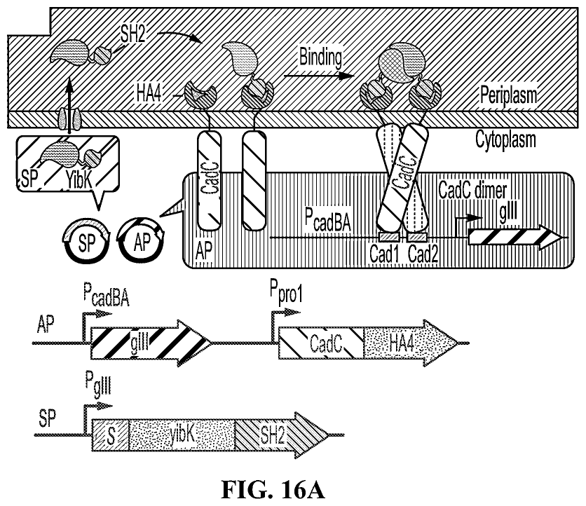

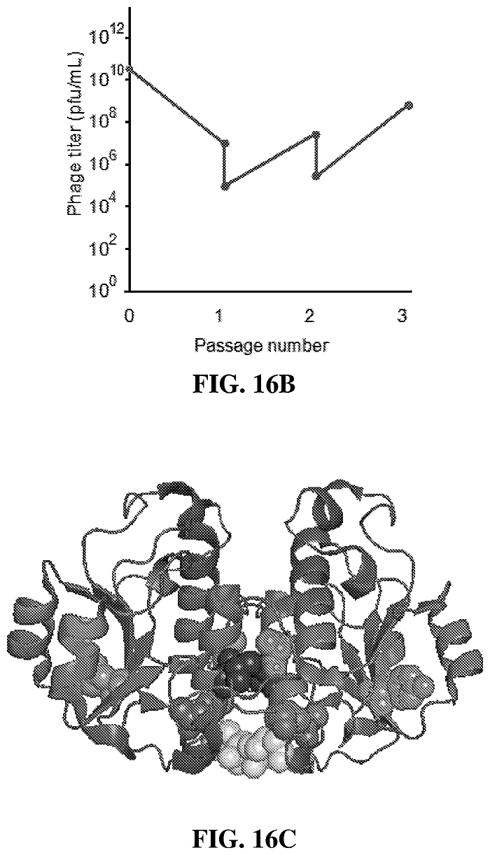

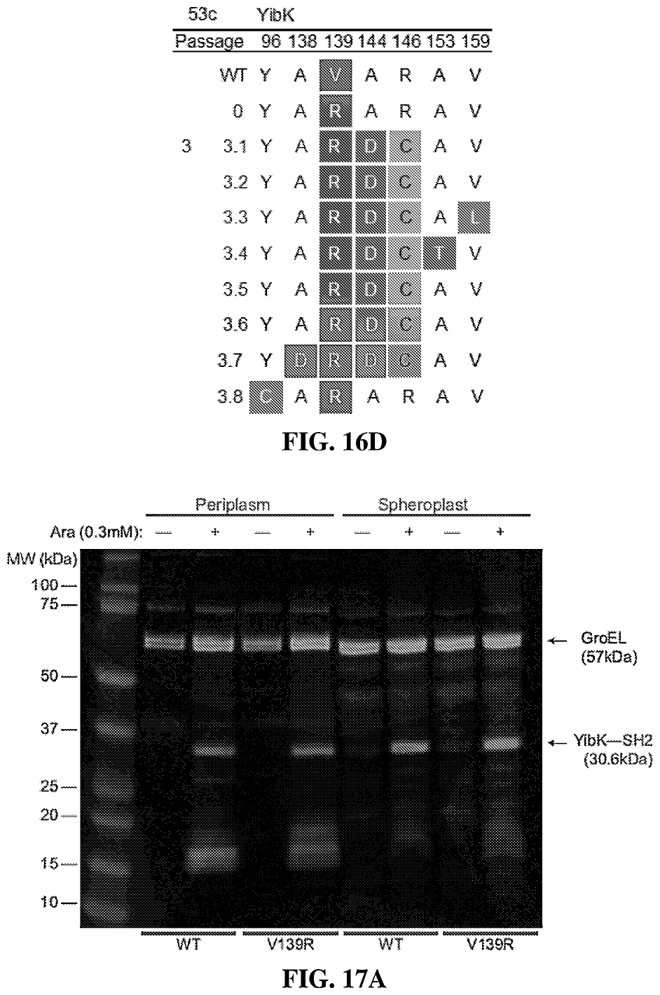

To validate the ability of the pPACE system to evolve periplasmic proteins that bind a protein target, the system was challenged to evolve homodimeric YibK variants if seeded with phage encoding the monomeric V139R variant. The pPACE system was adapted into the format of PANCE (phage-assisted non-continuous evolution), a non-continuous form of PACE in which cultures propagate phage in wells through multiple generations but undergo serial daily passaging in lieu of continuous flow, permitting a less stringent and more sensitive initial selection. After three PANCE passages, phage titers increased robustly (FIG. 16B).

Characterization of selection phage demonstrated that YibK variants evolved mutations that restore YibK dimerization. On the YibK dimer interface, V139 forms a hydrophobic contact with A138′ of its binding partner. Evolving phage did not directly revert the V139R point mutation, which requires two point mutations in the same codon. However, in PANCE-evolved clone 3.7, residue A138 mutated to an aspartic acid (GCC to GAT), completely restoring affinity as measured by P_{cadBA} transcriptional activation (FIG. 2D, 2F). It was also observed that R146, which forms an intermolecular salt bridge with E143′ of the counterpart YibK subunit in close proximity to R146′, was converted to a cysteine residue in seven of eight sequenced phage (CGT to TGT; FIG. 2G; FIG. 16D). Incorporating 146C in a nonbinding background results in stronger transcriptional activation of P_{cadBA} than wild-type YibK.

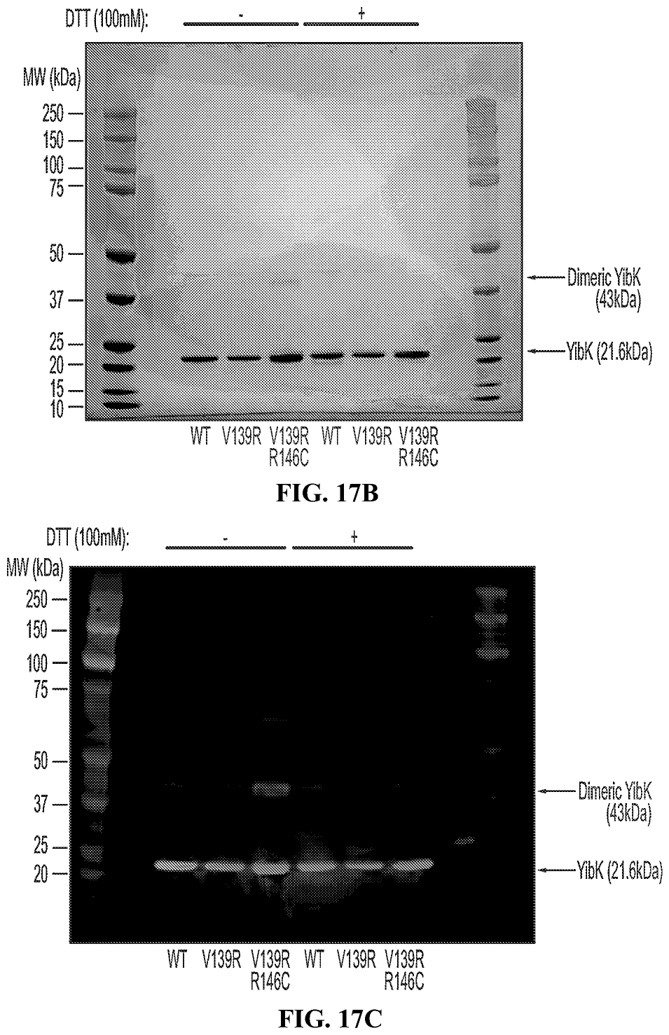

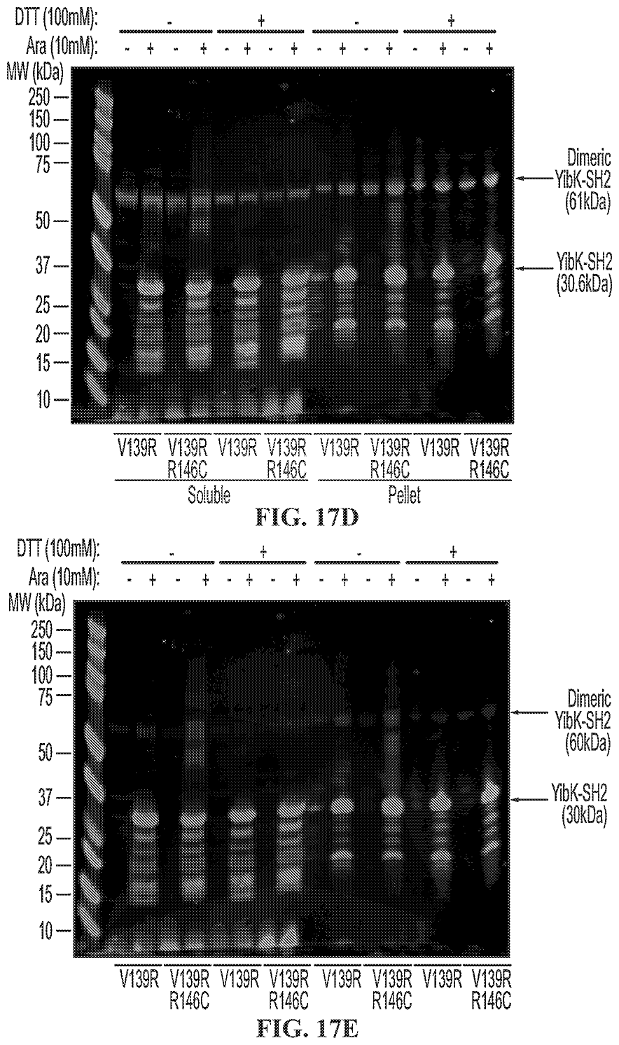

Remarkably, it was found that R146C results in an intermolecular disulfide bridge, visible by SDS-PAGE in purified YibK protein, as a ˜43 kDa band representing the dimeric form of the 21.6 kDA monomer (FIG. 2E, FIGS. 17B-17C). In whole-cell lysates, a ˜60 kDa band representing the dimer of 30 kDa YibK-SH2 can also be visualized (FIGS. 17D-17E). Both dimeric species are lost upon addition of reducing agent Together, these results establish that the periplasmic selection platform is capable of restoring and even improving stable homodimerization of a monomeric protein by multiple mechanisms, including the evolution of novel disulfide bridges absent in the native protein.

### Periplasmic Evolution of Antibody-Antigen Affinity

Next, it was sought to use pPACE to evolve antibodies that bind antigens of interest. Full-length monoclonal antibodies can be engineered into smaller forms such as single-chain variable fragments (scFvs). ScFvs comprise only the heavy and light chain variable antigen binding regions (V_{H} and V_{L} respectively) tethered by a flexible synthetic linker. ScFvs are small in size (˜30 kDa), can be produced in E. coli, and can be readily conjugated to drug molecules, effector proteins, and chimeric antigen receptors, making them prime candidate molecules for directed evolution approaches. Heterologous expression of scFvs in E. coli typically involves tagging them for export into the periplasm using an N-terminal signal sequence peptide.

pPACE was applied to evolve scFv forms of antibodies. To validate this capability, the Ω-graft antibody scFv was chosen, which targets the leucine zipper GCN4 with K_{d}˜500 pM. To determine whether an antibody-antigen interaction could drive CadC dimerization in the same way as a homodimeric YibK interaction, CadC-HA4 and Ω-graft-SH2 were expressed, with or without co-expression of a monomeric form of the leucine zipper GCN4 (GCN4(7P14P)) fused to SH2. In this architecture, the binding of Ω-graft to GCN4 drives dimerization of CadC-HA4 bound to Ω-graft-SH2 and a CadC-HA4 molecule bound to GCN4-SH2, creating a four-part complex (FIG. 3A). The presence of the GCN4 antigen led to a 30-fold increase in P_{cadBA}-driven LuxAB expression compared to when the antigen was absent (FIG. 3B). In contrast, substitution of a Ω-graft double point mutant L231F F232A, which impairs binding for GCN4 by >7,000-fold, in place of wild-type Ω-graft led to a 55-fold decrease in transcriptional activation (FIG. 5B). Collectively, these results indicate that the pPACE selection can link scFv target binding to transcriptional activation of P_{cadBA}.

To determine whether periplasmic PACE can distinguish between functional and nonfunctional forms of the Ω-graft scFv, a competitive mock-selection experiment was performed under both continuous and non-continuous flow conditions, without mutagenesis.

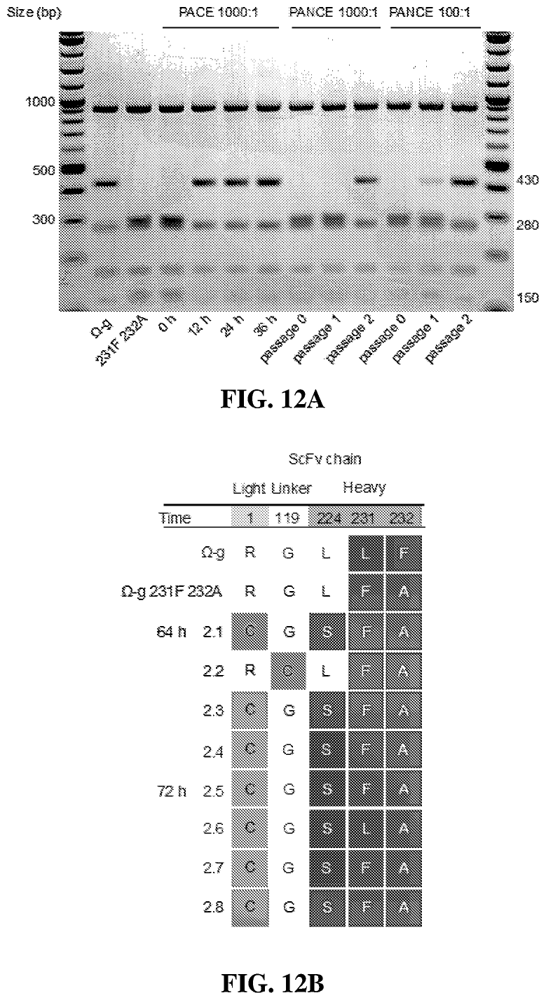

Host cells expressing CadC-HA4 and GCN4-SH2 and encoding P_{cadC}-driven gene III on the AP were seeded with a mixture of selection phage containing a 1:1,000 ratio of unmutated Ω-graft-SH2 selection phage to L231F F232A mutant-SH2 selection phage and PACE and PANCE were carried out. Within 12 hours of PACE or following two serial PANCE passages at a dilution factor of 1:100 per passage, unmutated Ω-graft variants dominated both populations, enriching ≥1,000-fold (FIG. 12A). These results demonstrate that the selection platform and activation of transcription at P_{cadBA} can be used to selectively propagate phage encoding a target-binding antibody scFv during pPACE and pPANCE.

Regulating scFv Periplasmic Export

In the small volume of the periplasmic space, minor changes in protein expression level can have a large impact on relative concentrations. An evolving SP encoding an scFv might achieve increased fitness during pPACE by modifying the promoter driving scFv expression to raise the effective dose of scFv in the periplasm to compensate for a low (e.g., poor) Kp. It was reasoned that controlling scFv export to the periplasm would be desirable to maintain selection for pressure. Further, regulating the level of periplasm-targeted scFv protein could in principle drive two simultaneous selections: for high affinity to the target to overcome low effective concentration of scFv; and for increased solubility of the scFv, to raise effective concentration of scFv. Therefore, a key aspect of a related PACE selection that was recently reported, soluble expression PACE or SE-PACE, was adapted to integrate two signals within a PACE selection. SE-PACE uses a trans-splicing split intein to reconstitute two signal sequence fragments into a single functional protein, integrating transcription from two promoters into one output. In SE-PACE, intein-mediated splicing reconstitutes the signal sequence peptide of pIII, which must enter the periplasmic space for phage to exit the host cell in an infective form, demonstrating that protein export into the periplasmic space can be regulated using inteins.

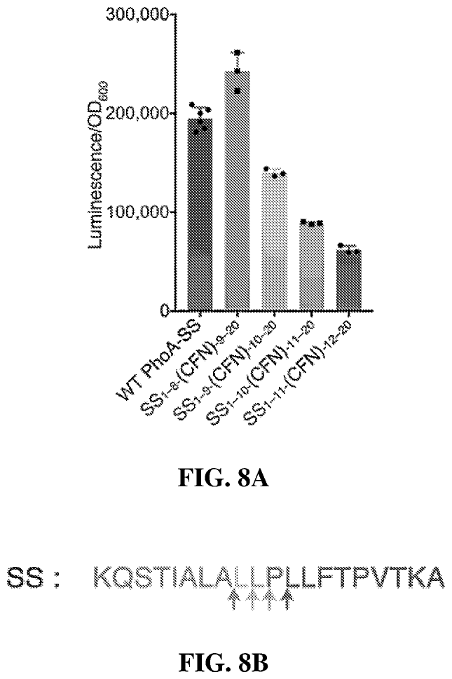

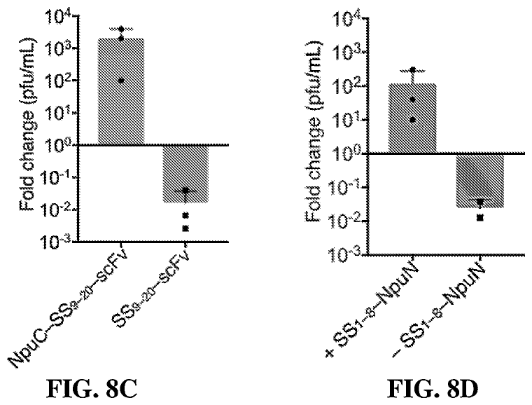

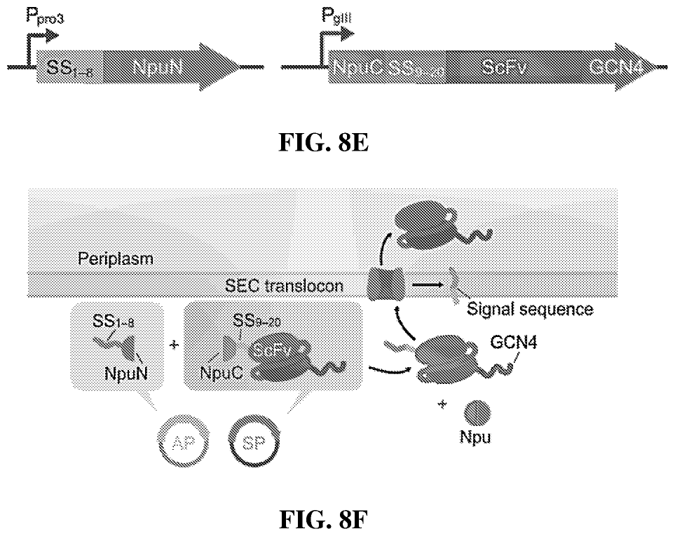

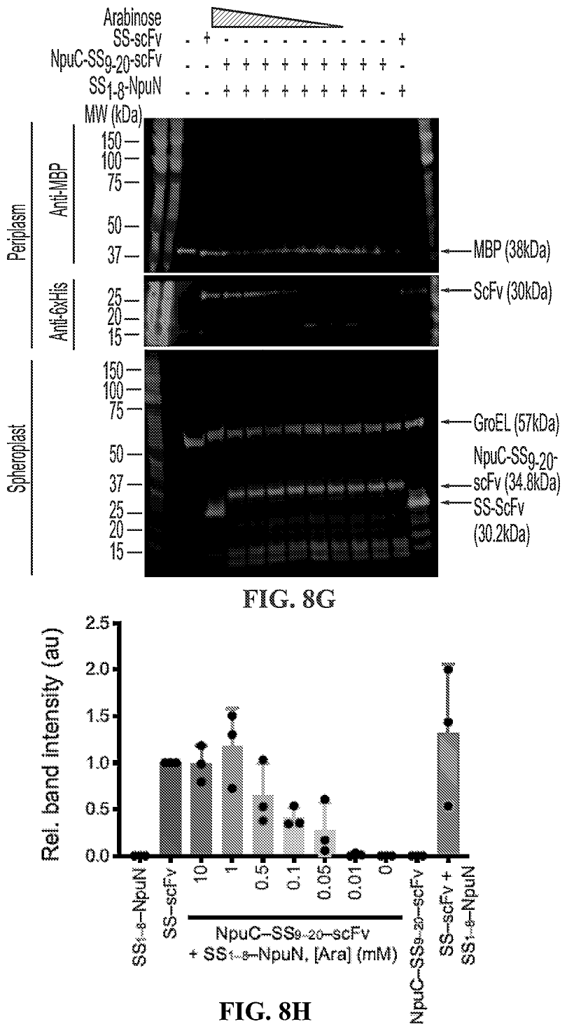

The phosphatase A (PhoA)-derived signal sequence (SS) used to direct protein export into the periplasmic space was split into two halves, consisting of signal sequence amino acids 1-8 and 9-21 (FIGS. 8A-8H). These two halves were fused, respectively, to the N- and C-terminal portions of the Nostoc punctiforme (Npu) trans-splicing DnaE intein. SS amino acids 1-8 were fused to the N-terminal half of the Npu intein on a host AP1, inhibiting phage from evolving increased expression of this component. The C-terminal half of the Npu intein, fused to SS amino acids 9-20 and the evolving scFv-SH2 fragment, was encoded on the selection phage. Following translation of both fusion proteins, intein-mediated splicing reconstitutes the full-length SS fused to the evolving scFv, allowing SS-directed periplasmic export (FIGS. 8A-8H).

Using the Ω-graft pPACE selection described above, it was observed that expressing Ω-graft-SH2 with its SS split into two polypeptides, each fused to each half of the Npu intein, led to pIII expression and robust phage propagation, indicating that the signal sequence could be reconstituted in E. coli and could direct Ω-graft-SH2 export to the periplasm.

In contrast, when the C-terminal domain of Npu was omitted from the SS₉₋₂₀-Ω-graft-SH2 construct, phage failed to propagate (FIG. 8C). Similarly, the expression of the SS₁₋₈-NpuN construct is necessary for propagation of NpuC-SS₉₋₂₀-Ω-graft-SH2 phage (FIG. 8D). It was further found that by expressing SS₁₋₈-NpuN under small molecule induction in the presence of NpuC-SS₉₋₂₀-Ω-graft (34.8 kDa), periplasmic expression of Ω-graft scFv (30.2 kDa) could be driven in a dose-dependent manner (FIGS. 8G-8H). This demonstrates that split inteins (e.g., split Npu inteins) can regulate reconstitution of full-length SS for periplasmic export of scFvs and P_{cadBA} activation.

Under this intein-regulated system, the total amount of scFv exported to the periplasm, and thus available to fold, bind to antigen, and direct CadC dimerization, is limited by the availability of the intein-SS fragment encoded on the host AP. The scFv can only enter the periplasm following reconstitution of full-length SS-scFv from the phage-encoded fragment and the host-encoded fragment. The researcher can modify the strength of the promoter driving intein-SS₁₋₈ fragment expression level of intein-SS1-8 fragment (e.g., on an AP) to limit the reconstitution of full-length SS-scFv, and thus limit the amount of scFv exported to the periplasm, independent of evolution of the promoter driving intein-SS₉₋₂₀-scFv expression. Thus, scFv concentration can be made limiting, creating selection pressure for efficient expression of soluble scFv as well as increased selection pressure for high affinity to compensate for low effective scFv concentration.

Evolution of the Ω-Graft Antibody and Overcoming scFv Homodimerization

Next, pPACE was challenged to correct the L231F F232A binding mutation in the Ω-graft antibody scFv, using both a traditional full-length N-terminal SS sequence and the intein-SS strategy described above, in order to select for affinity alone as well as affinity and soluble periplasmic expression. It was aimed to apply pPACE to restore binding to GCN4 by correcting mutations L231F and F232A in a pPACE selection.

PACE experiments using the original selection architecture (FIG. 3A) resulted in two classes of genotypic outcomes. First, close to half of phage reverted mutation L231F to the wild type within 96 hours of pPACE. Second, scFv variants developed cysteine residues at their N-termini or within the 4×GGGS (SEQ ID NO: 43) linker connecting scFv VH and VL domains (FIG. 3C). Linker cysteines in particular appeared mutually exclusive to the desired L231F reversion (FIG. 12B). Point mutations were examined in isolation in an L231F/F232A background and found that at both positions, a cysteine substitution resulted in higher transcriptional activation than reversion at position 231 to Leu (FIG. 3D). The insertion of a C-terminal Cys residue has been used to manufacture stable dimeric scFvs through formation of a covalent disulfide. It was reasoned that an N-terminal or linker Cys residue might form a similar covalent linkage, generating stably homodimeric scFv-SH2. Such an interaction would circumvent the target-binding selection by forming a stable scFv-SH2 homodimer, with both SH2 domains available to bind two CadC-HA4 molecules and bring them into close proximity, without involvement of the antigen (e.g., by improving the target antigen binding affinity of the scFv) (FIG. 3E).

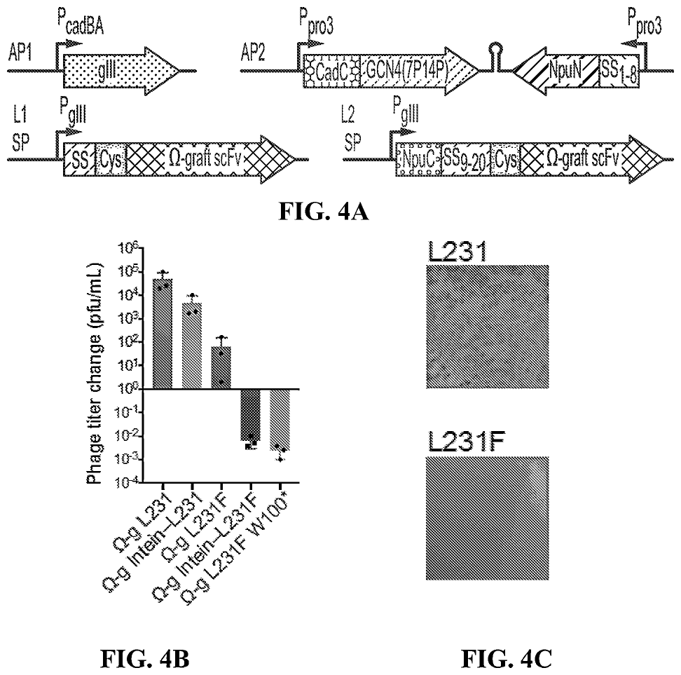

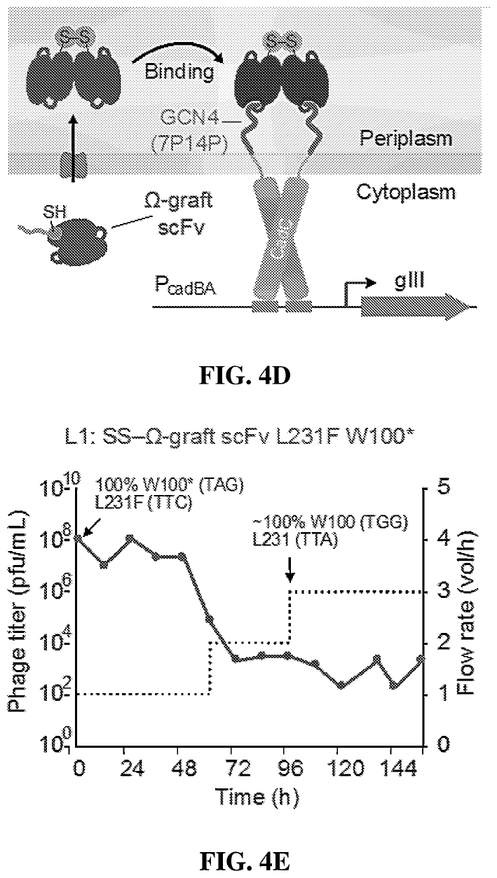

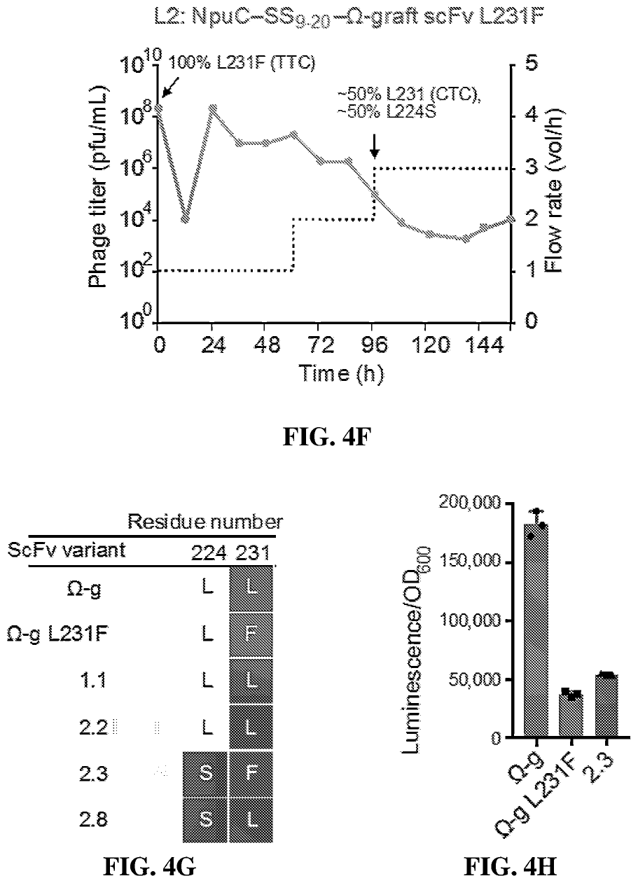

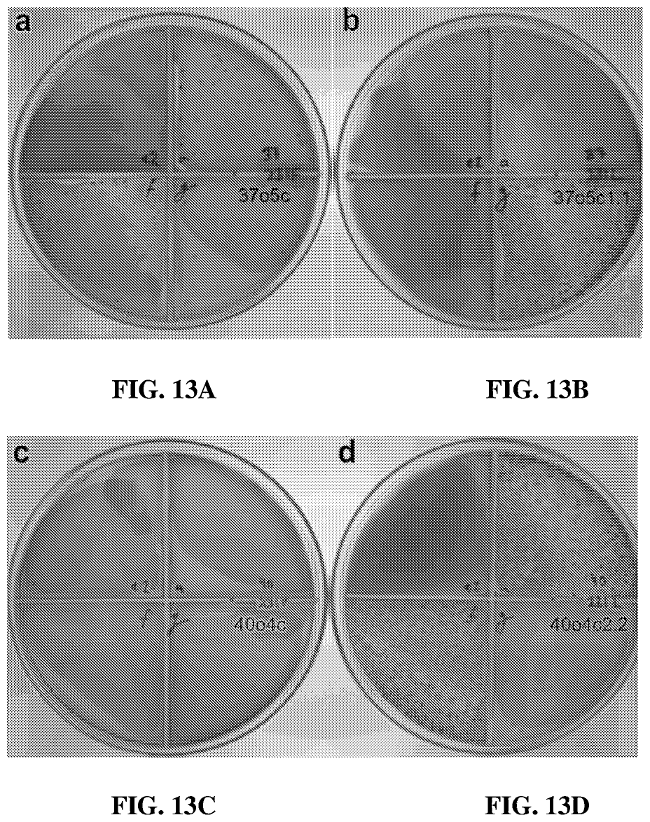

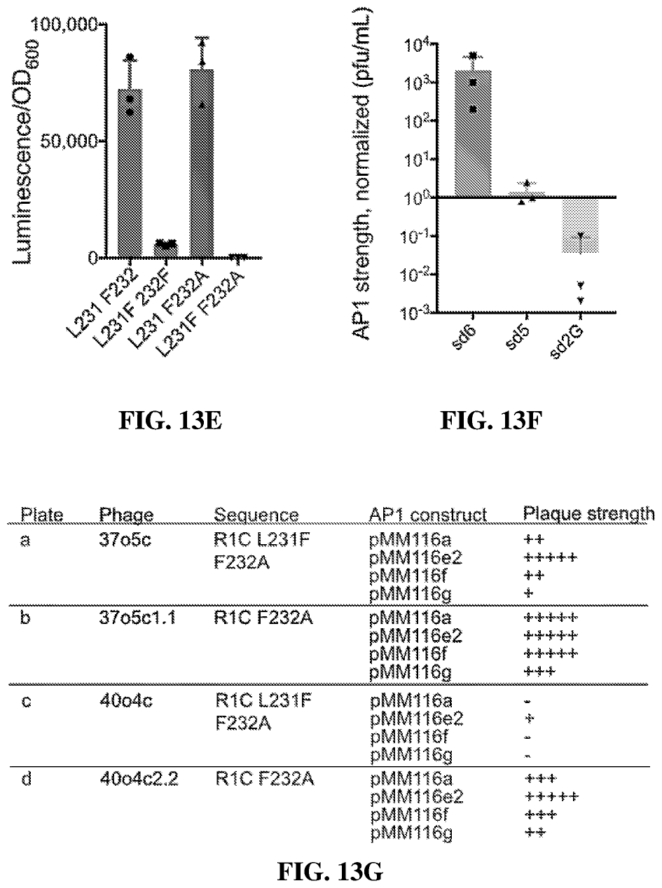

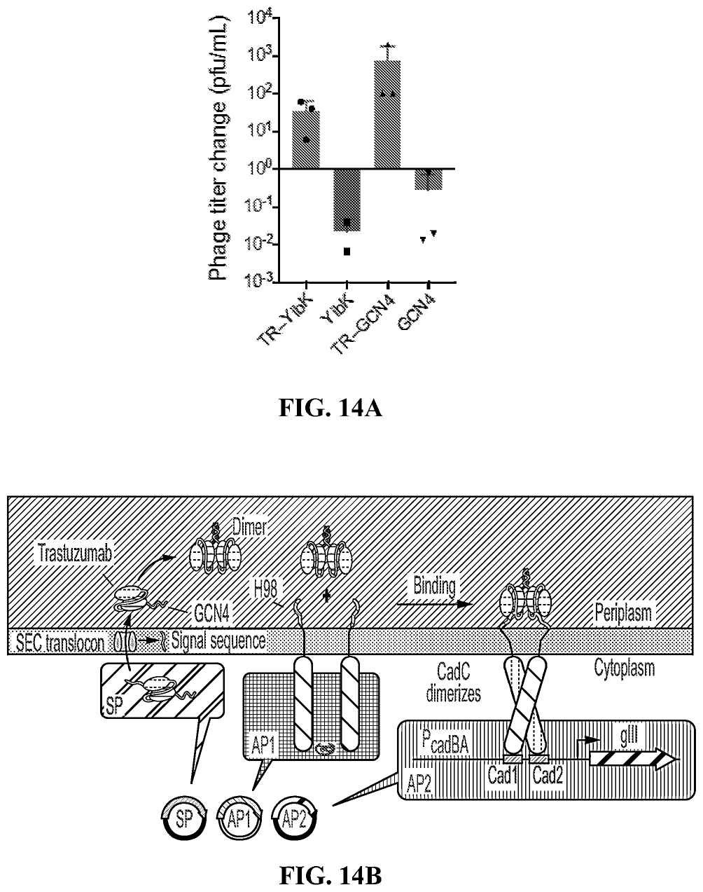

To prevent circumvention of the target-binding selection, the selection architecture was modified by fusing the GCN4(7P14P) antigen directly to CadC in place of HA4, to eliminate the possibility of scFv homodimerization resulting in selection survival (FIG. 3F). Obligate homodimeric scFvs were created by removing the now-redundant SH2 domain fusion and either pre-installing an N-terminal cysteine in the Ω-graft scFv (FIG. 4A), or, as a more general strategy, by fusing a homodimerizing GCN4 leucine zipper domain C-terminal to the scFv (FIG. 3F; FIG. 5A; FIGS. 14A-14B). This strategy ensures that efficient dimerization does not depend on the properties of the scFv being evolved, since different scFvs homodimerize at different rates. In this second-generation selection architecture, a dimeric scFv antibody must bind two CadC-fused antigens to activate P_{cadBA}. Transcriptional assays indicated that mutation L231F is primarily responsible for loss of binding and that F232A alone has little effect on binding (FIG. 13E). Therefore, reversion of F232A was considered to be unnecessary in desired selection outcomes.

Using this second-generation architecture, phage encoding canonical Ω-graft showed three orders of magnitude higher levels of propagation in overnight enrichment assays than phage encoding Ω-graft L231F (FIGS. 4B, 4C). Incorporation of a nonsense mutation in the Ω-graft scFv at position 100 (W100*) also led to strong de-enrichment of phage (FIG. 4B).

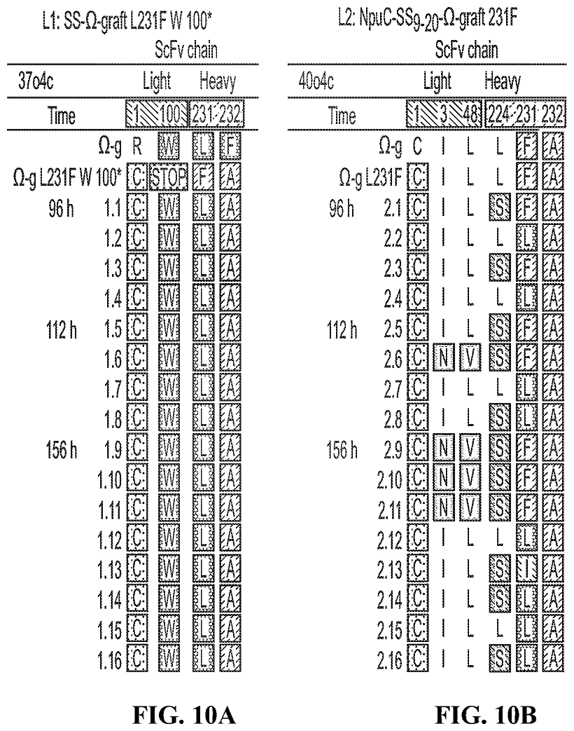

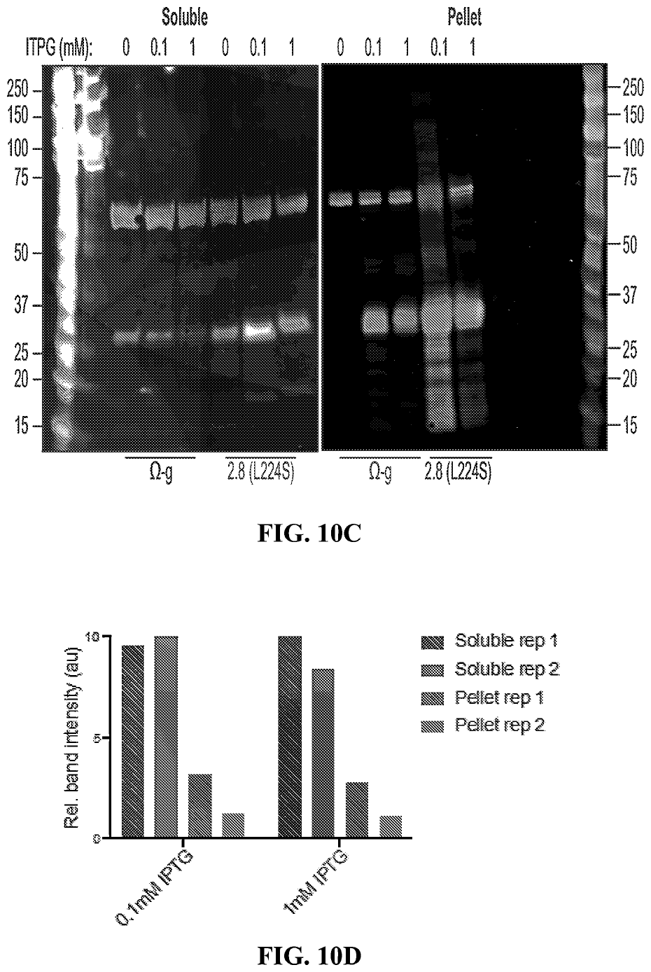

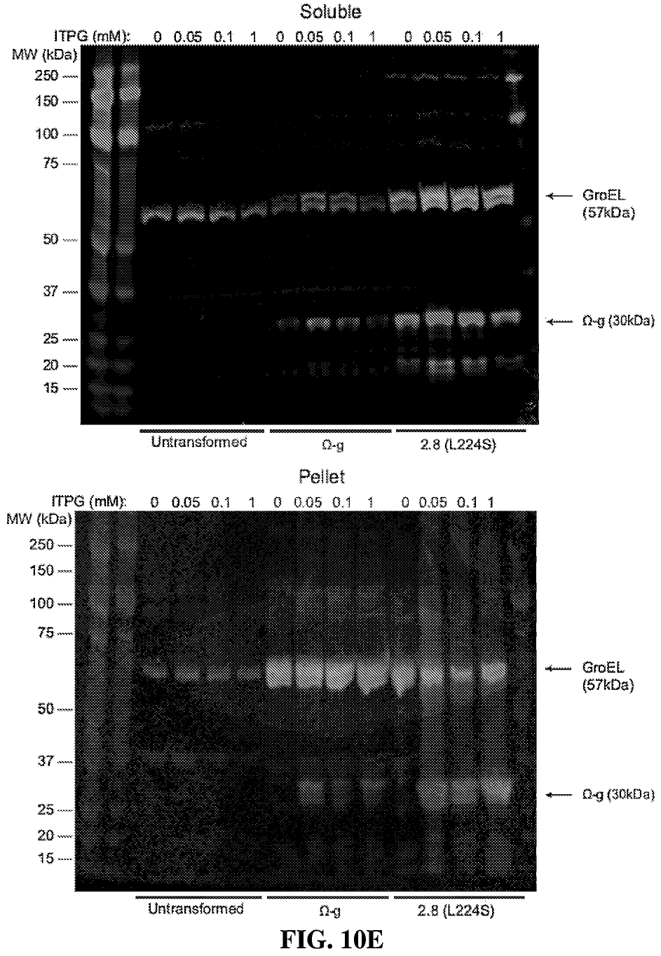

pPACE was challenged using the second-generation architecture to correct a stop codon at W100 in addition to the L231F binding defect mutation. Within 96 hours of pPACE, phage populations fully reverted mutations correcting both deleterious mutations in population 1 (FIG. 4E, FIG. 10A). In population 2, the split intein signal sequence strategy described above was used to regulate periplasmic scFv expression in host cells (FIGS. 4A, 4F). Due to the decreased fitness of intein-SS phage compared to phage with full-length SS (FIG. 4B), population 2 was not challenged to correct a stop codon. Mutation F231L was present in ˜50% of this population by 96 hours and dominated the population by 156 hours (FIG. 10B). Phage in different populations accessed leucine codons at position 231 via two distinct point mutations, converting TTC (Phe) to TTA (Leu) in population 1 and to CTC (Leu) in population 2 (FIGS. 4E-4F). Importantly, no new cysteines arising during evolution were observed (FIG. 4G, FIG. 10A). These results indicate that the second-generation pPACE selection prevents phage from passing the selection by evolving stable scFv-SH2 homodimers alone, and requires a tight scFv-antigen interaction in order to activate P_{cadBA}.

In population 2, which utilized the intein-SS strategy, enrichment of two point mutations, F231L and L224S, were observed as separate solutions present at similar frequency at 96 hours. Mutations F231L and L224S were observed together in the same variant by 112 hours (FIG. 10B). Mutation of L224V was previously reported to enhance the cytoplasmic solubility of the Ω-graft scFv³⁸. Soluble expression of Ω-graft scFv and variant L224S were compared by conducting a small-scale expression of both variants with an N-terminal PhoA SS from the promoter P_{T7Lac} in BL21*DE3 cells by western blotting. Relative expression levels were determined by Western blot and normalized to a reference band, the housekeeping protein GroEL. It was found that L224S increased soluble expression of Ω-graft by roughly 8-fold (FIG. 10C-10E).

Together, these findings demonstrate that pPACE can restore affinity of an antibody to an antigen, and that regulating periplasmic export of the evolving species using a split-intein signal sequence can support the evolution of improved soluble expression as well as improved binding. These results also show that the second-generation pPACE system can avoid the evolution of outcomes that circumvent the selection by homodimerizing the evolving protein, rather than by binding the target.

Periplasmic PACE of Novel Trastuzumab scFv Variants

The second-generation pPACE selection was used to evolve an scFv form of the antibody trastuzumab (Herceptin), to bind a new target antigen. Trastuzumab targets the oncogenic receptor tyrosine kinase Her2 and is a successful first-line treatment for Her2⁺ breast cancers. Most trastuzumab-responsive tumors however develop resistance to the drug within one year. Second-line treatments can overcome resistance using multi-specific engineered antibodies, which combine variable domains of two or more mAbs with effector domains to generate antibodies that target several epitopes simultaneously, including bispecific antibodies that also target Her3, EGFR, and VEGF kinase receptors. The ability of pPACE to rapidly evolve affinity to novel epitopes could further broaden the targeting capacity of engineered multi-specific antibodies.

The Her2 mimetic peptide H98 was identified in a peptide library screen for trastuzumab binding. H98 bears structural similarity but no sequence homology to Her2. Mimetic peptides (mimotopes) such as H98 are of interest to generate vaccines which can focus an immune response towards a single relevant antigen, minimizing the likelihood of eliciting an autoimmune response from cross-reactivity with related self-proteins. Mimetic peptides have shown promise in vaccines targeting Her2, VEGF, and PDI and viruses such as respiratory syncytial virus and HIV. H98 has been considered for use as a mimotope to induce trastuzumab-like antibodies for cancer treatment. Immunization with GST-fused H98 successfully elicited Her2-responsive antibodies in BALB/c mice.

It was sought to apply pPACE to evolve an scFv form of trastuzumab with higher affinity for the H98 peptide. Trastuzumab scFv was evolved in the second-generation pPACE selection using either full-length SS or the split intein SS strategy, resulting in mutually exclusive outcomes within 96 hours of evolution. The H98 peptide antigen was presented as a CadC-H98 fusion driven by a weak constitutive promoter on the AP, such that a small but stable pool of CadC-H98 was available on the inner membrane for scFv binding. Trastuzumab was expressed as an scFv-GCN4 fusion to ensure dimerization, as it was found that use of a larger domain such as YibK to direct dimerization resulted in poor phage propagation (FIG. 5B), possibly due to excessive crowding of the SEC translocon or the periplasmic space.

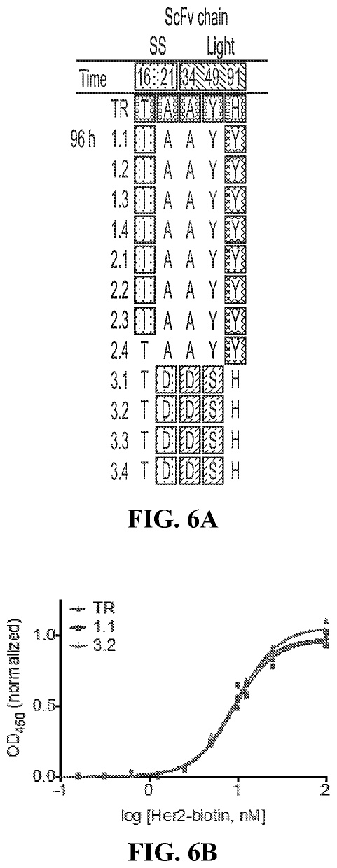

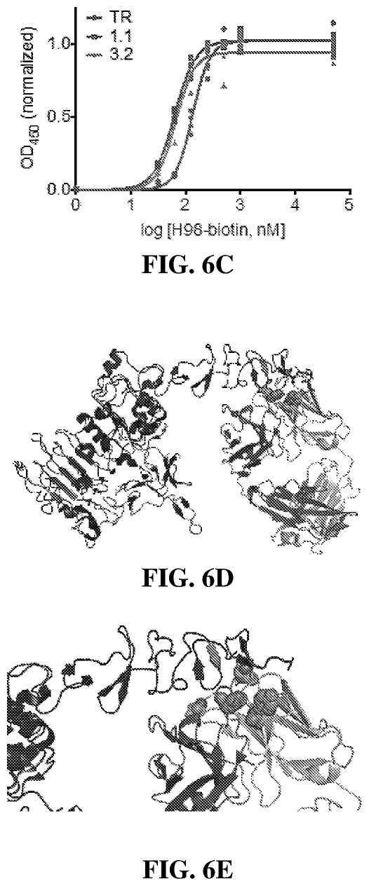

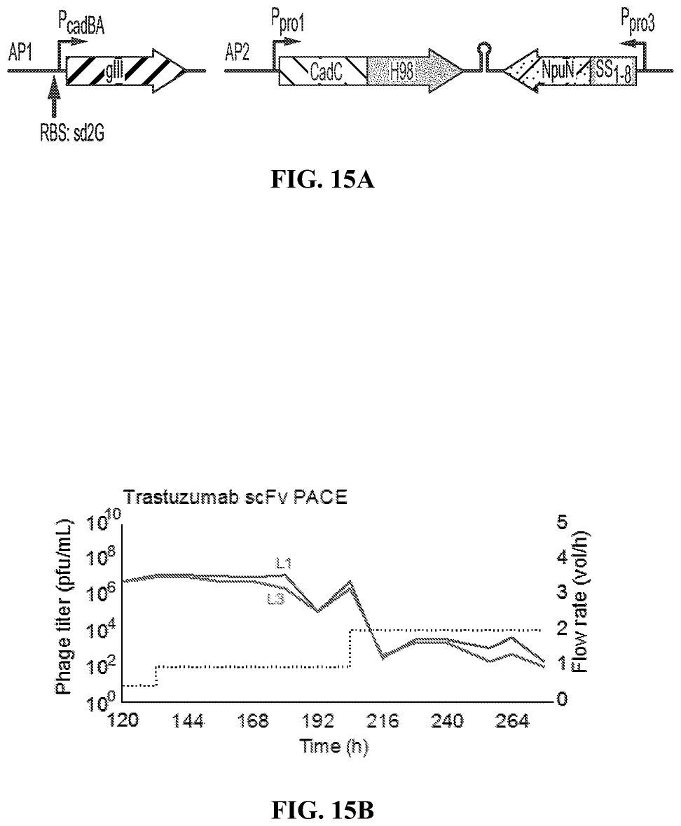

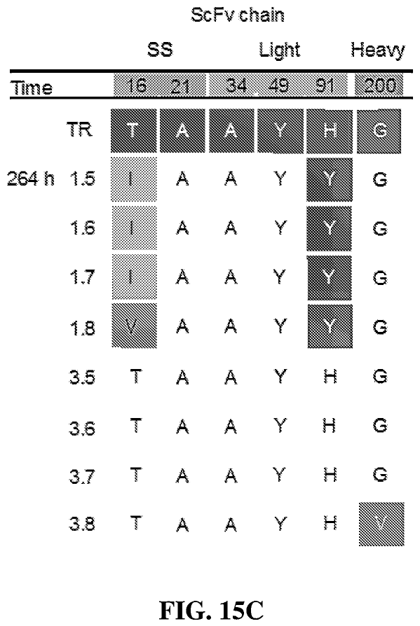

Phage were allowed a 24-hour period of evolutionary drift when pIII was provided freely in combination with elevated mutagenesis³⁰ to generate a large and diverse phage library. Phage were then subjected to a high-stringency pPACE selection at increasing flow rates until titers plateaued (FIG. 5C, FIG. 6A-6E). In populations 1 and 2, phage encoded the full-length signal sequence. Both populations converged on a single point mutation, H91Y (variant 1.1, FIG. 5D). In population 3, periplasmic export was restricted through the split intein strategy described above, leading to enrichment of a single variant (3.2) with mutations A34D Y49S. Periplasmic PACE experiments carried out at additional increased stringencies by reducing both antigen availability and pIII translation levels did not result in the enrichment of any additional point mutations in the scFv (FIGS. 15A-15C).

Computational modeling indicates that trastuzumab interacts with H98 through heavy chain residues V33, R50, and Y105, and light chain residues T94 and N30. In the trastuzumab crystal structure, residue T94 is proximal to residue H91 (H91Y in variant 1.1), and residue N30 is proximal to residue A34 (A34D in 3.2) (FIG. 6E). Light chain residue Y49 is adjacent to residue A34 in β-sheet, and mutation Y49S (variant 3.2) may help to accommodate the substitution of alanine for a relatively bulky, charged aspartic acid at position 34 (PDB ID: 1N8Z⁸⁷).

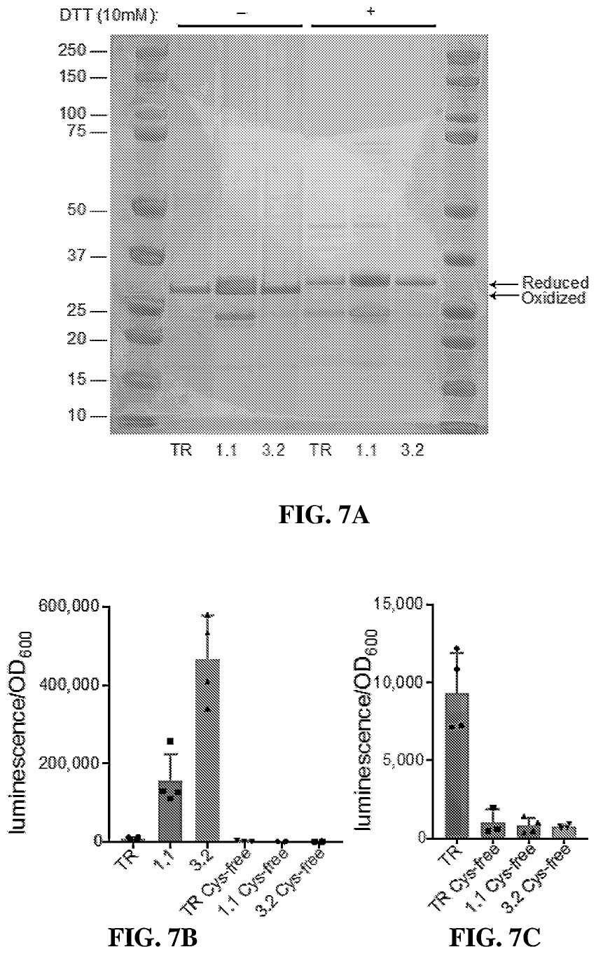

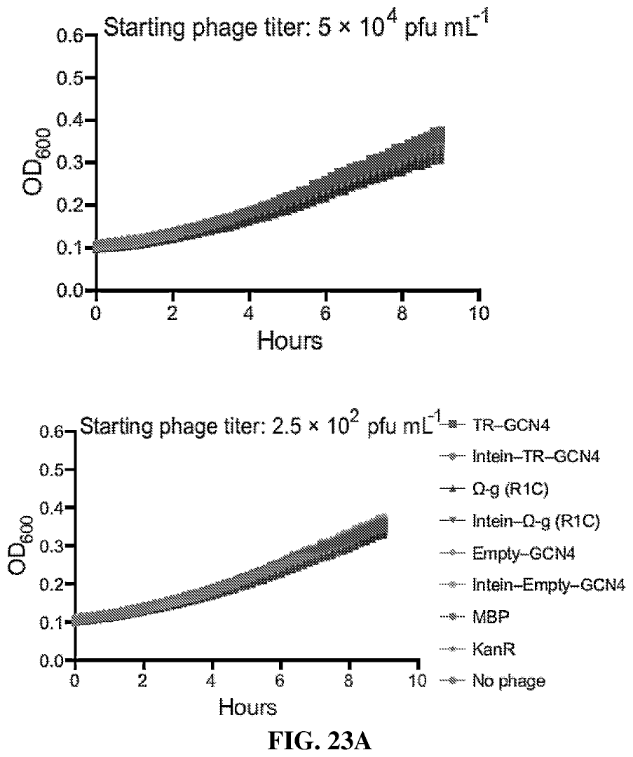

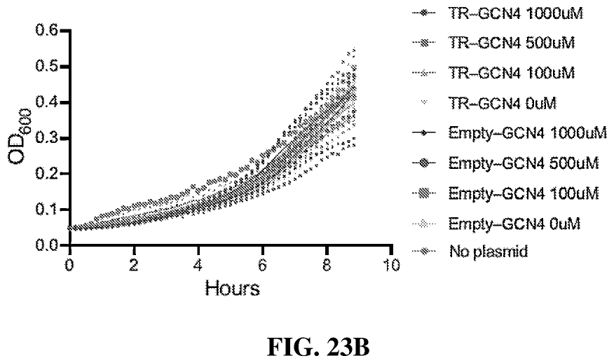

ScFvs migrate more rapidly when intra-chain disulfide bonds are intact than when they are reduced to free thiols. Trastuzumab and evolved variants show a similar, characteristic change in mobility consistent with reduction of disulfides during SDS-PAGE under reducing conditions compared to oxidizing conditions, indicating that the trastuzumab intra-chain disulfides are retained in evolved variants (FIG. 7A). To examine the role of intra-chain disulfides in the stability and binding of trastuzumab and evolved variants, the possibility of disulfide formation in trastuzumab was abrogated and variants were evolved by replacing the disulfide-forming cysteine residues with serine amino acids. In the absence of disulfide bonds, both trastuzumab and evolved variants failed to induce transcription from P_{cadBA} (FIG. 7B). These results further indicate that trastuzumab binding is likely dependent on intra-chain disulfides, in agreement with the findings of Wörn and Plückthun that expression of trastuzumab scFv without disulfide bonds results in insoluble protein⁸⁹, and that these disulfides are preserved through pPACE. To prevent accumulation of insoluble cytoplasmic scFv from impairing host cell fitness, a growth time-course was carried out, and it was found that scFv expression, with or without split-intein SS, had little to no effect on host cell growth (FIGS. 23A-23B).

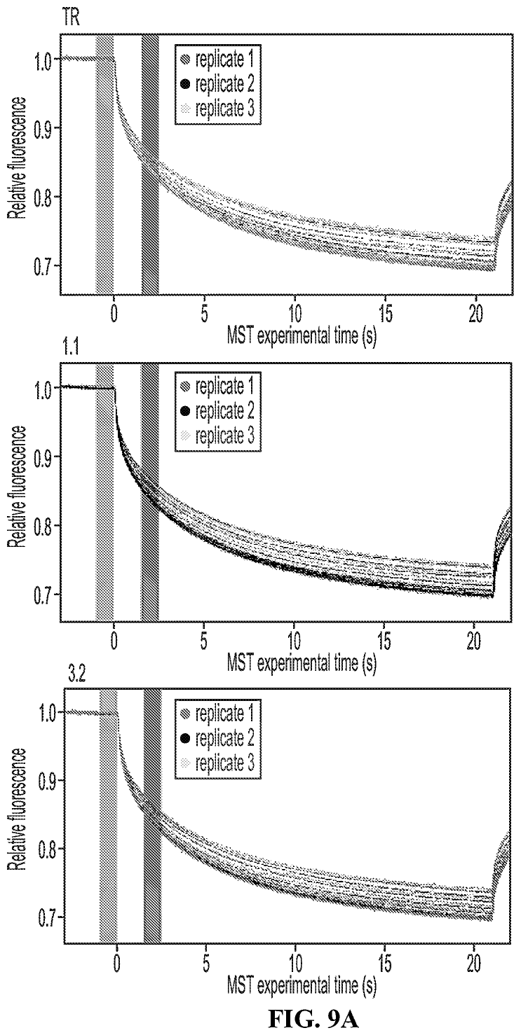

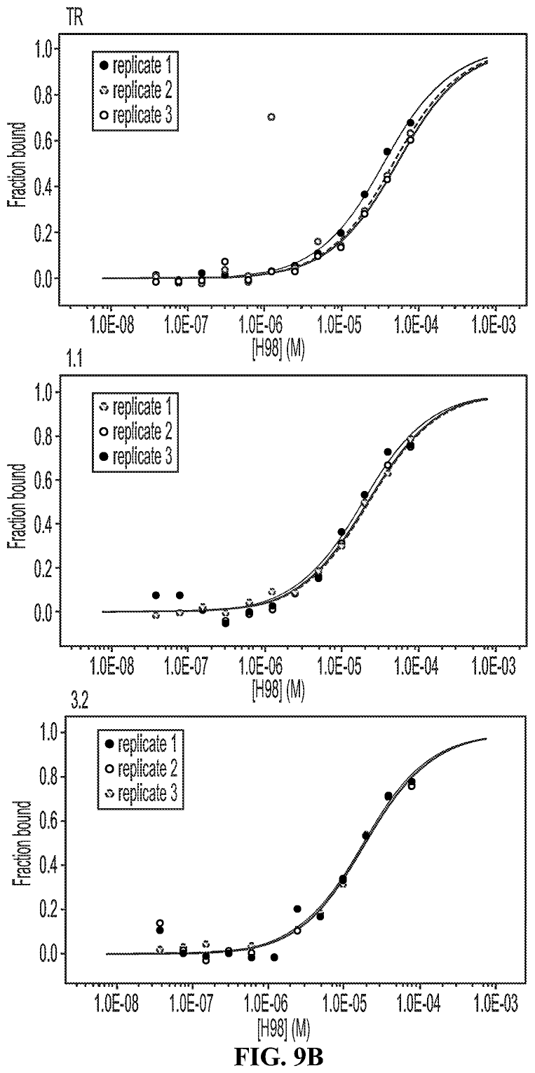

In both populations that did not use the split-intein SS selection, evolved variant 1.1 dominated the evolved outcomes. This variant showed ˜2.5-fold improved binding to H98 by both ELISA and MST and little change in soluble expression (FIGS. 5F-5H, FIGS. 9A-9B, FIGS. 11C-11D). Evolved variant 3.2 was selected using the split intein SS selection and showed a ˜2-fold increase in affinity (FIGS. 5F-5H, Table 1, FIGS. 9A-9B). To support ELISA data, microscale thermophoresis (MST) was also carried out (FIGS. 9A-9B, Table 6). It is noted that in MST experiments, the upper bound of the binding curve was not accessible due to solubility limits of the H98 peptide in aqueous buffer (FIGS. 9A-9B), which may affect accuracy of K_{d} determination. Thus, calculations of EC50 and K_{d} are provided from MST and EC₅₀ from ELISA separately (Table 1). EC₅₀ values are not directly comparable between MST and ELISA, due to differences in scFv concentration and spatial organization of antigens, but reflect similar fold improvements for evolved variants over the starting scFv.

TABLE 1
Properties of trastuzumab scFv and evolved variants.

| | EC₅₀ (nM)ª | Hill slope^{a} | EC₅₀ (μM)^{b} | K_{D} (μM)^{b} | T_{M} (C°) |
|---|---|---|---|---|---|
| TR | 160.7 ± 26.2 | 3.1 ± 0.6 | 4.3 ± 1.6 | 44.9 ± 8.7 | 68.5 |
| 1.1 | 63.6 ± 3.0 | 2.6 ± 1.4 | 1.4 ± 1.1 | 20.5 ± 1.9 | 72.5 |
| 3.2 | 77.6 ± 15.0 | 3.0 ± 1.0 | 1.3 ± 1.1 | 18.8 ± 0.7 | 62.5 |

Trastuzumab is abbreviated as TR.
ªValues were determined by pooling means from four ELISA experiments conducted with separate protein preps, each with four technical replicates per ELISA experiment, and calculating mean and s.d. of pooled means.
^{b}Values reflect mean and s.d. of three technical replicates in MST (FIGS. 9A-9B). Melting temperature data reflects mean of two experiments conducted with separate protein preps, each consisting of four technical replicates.

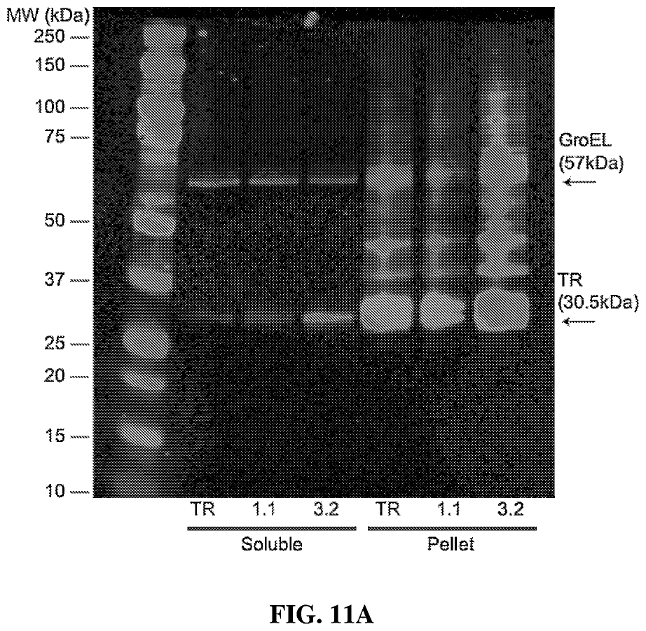

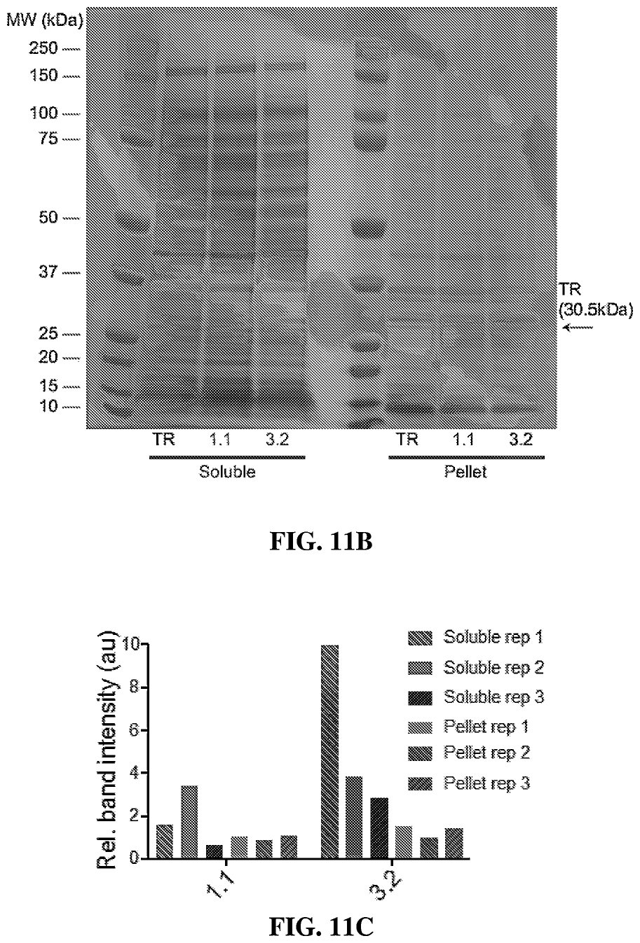

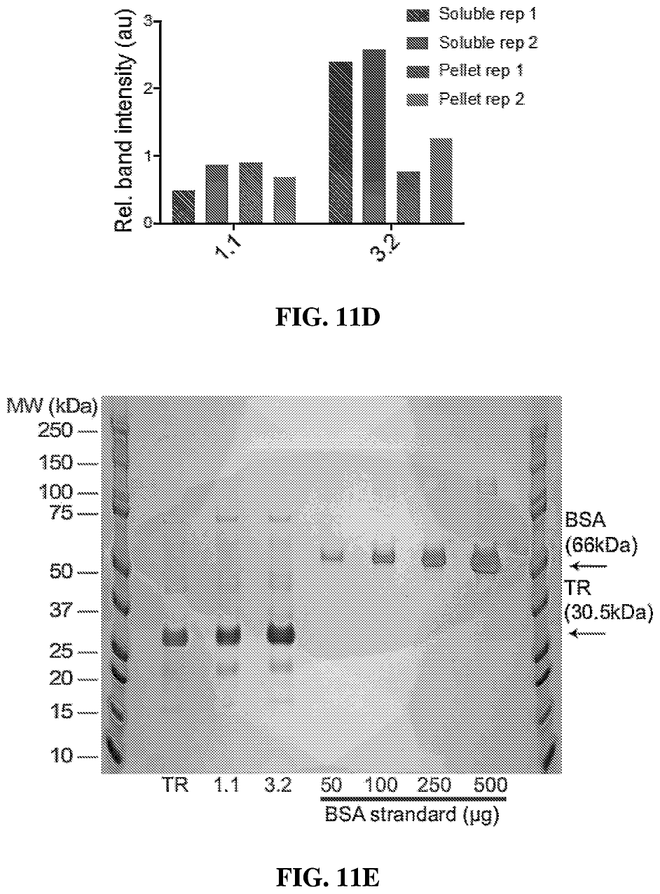

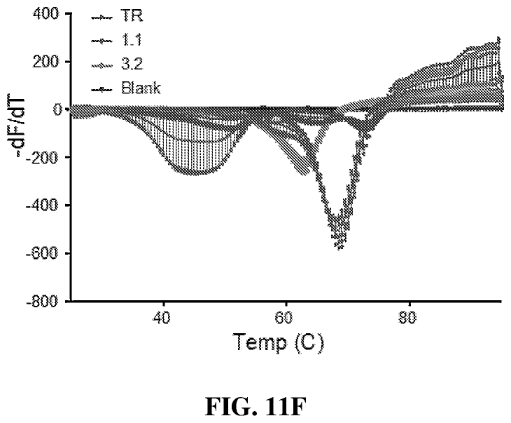

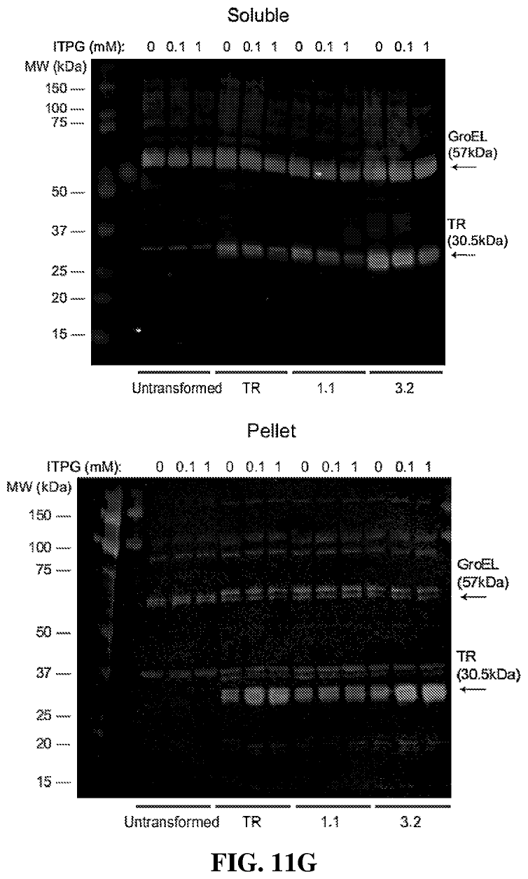

Variant 3.2 also showed substantial increases in soluble periplasmic expression levels (˜5-fold as measured by western blotting and 2.5-fold as measured by less-sensitive Coomassie staining of whole-protein lysates; see FIGS. 11A-11G), indicating that restricting the level of scFv export to the periplasm selected for enhanced solubility to raise the effective concentration of antibodies in the periplasm. Evolved variants showed unchanged binding to Her2 in ELISA compared to that of trastuzumab scFv (FIG. 6B). The pPACE-evolved variants showed similar, relatively unchanged thermal stability compared to that of the normal trastuzumab scFv. Unevolved trastuzumab scFv had a melting temperature of 68.5° C., consistent with literature values of 68-72° C.^{90,91}. T_{M} increase of +4.0° F. or variant 1.1 and a T_{M} decrease of −5° C. for variant 3.2 were observed (FIG. 11F, Table 1).

Continuous directed evolution has the potential to significantly streamline antibody development, but disulfide-containing proteins represent a significant challenge for current continuous evolution methods, which occur in the reducing environment of the cytoplasm. A method for the continuous evolution of protein binding was developed that takes place in the bacterial periplasm. Periplasmic PACE can rapidly generate proteins with improved binding and expression properties from a starting gene within several days of evolution. Periplasmic PACE supports native disulfide bonds, which can be critical for the folding and stability of scFvs and other proteins in both prokaryotic and eukaryotic contexts. Splitting the signal sequence directing periplasmic export into two halves, with one half expressed at a controlled level on a host plasmid, allows the researcher to define the extent of export to the periplasm, thereby providing a way to directly modulate selection stringency in the periplasm.

pPACE was applied to evolve YibK variants with restored binding via two novel mechanisms in only three serial passages, Ω-graft antibody variants with restored binding and 8-fold improved solubility within 96 hours of pPACE, and trastuzumab variants with up to 5-improved solubility and 2.5-fold improved binding affinity to a peptide antigen within 96 hours of pPACE. Taken together, these studies establish that pPACE can evolve improved binding and expression profiles of antibodies and other proteins in the periplasmic space on short timescales.

In an oxidizing environment such as the extracellular space, intra-chain disulfides are highly conserved among natural proteins, and can make the ΔG of folding more favorable by 4-5 kcal/mol, corresponding to an increase in folded states over unfolded states of roughly three orders of magnitude. For non-intrabody applications such as CAR-T therapy, engineering disulfide-free scFvs is generally not desirable or necessary. Periplasmic PACE therefore offers a complementary strategy to other intracellular evolution methods by enabling continuous evolution for binding activity and soluble expression while conserving native disulfide linkages.

The properties of the periplasm offer opportunities that pPACE is well-suited to exploit. Protein channels in the outer membrane of E. coli render the periplasm permeable to water, ions, and hydrophilic solutes up to ˜600 Da in size. Further, the pH of the periplasm mirrors the pH of the extracellular environment. Composition of the growth medium used in pPACE may strongly influence the folding and activity of evolving proteins. pPACE may be used in the evolution of proteins with unusual pH requirements, and could be leveraged for applications involving small-molecule substrates.

Evolution towards peptide antigens (e.g. GCN4, 0.4 kDa) and YibK (21.6 kDa) has been shown herein. In some embodiments, first-generation architecture is appropriate for use with monomeric evolving proteins, while second-generation pPACE is appropriate for dimeric evolving proteins and antigens that can tolerate an N-terminal fusion.

ScFv phage with split-intein signal sequence propagated less robustly than their full-length SP counterparts (FIGS. 4B, 5B), likely due to reduced levels of periplasmic scFv. Under intein-regulated conditions only, both Ω-graft and trastuzumab phage evolved improved soluble periplasmic expression. These outcomes are consistent with a model in which limiting the rate of periplasmic export imposes selection pressure for both soluble periplasmic expression and affinity. In a regime limited by the availability of the intein-linked SS₁₋₈ (NpuN-SS₁₋₈) fragment, mutations that only improve overall expression levels are unlikely to have a large effect, as excess intein-SS₉₋₂₀-scFv construct simply accumulates in cytoplasm. Notably, increased scFv in the pellet fraction, which represents insoluble protein, was not observed following intein-regulated pPACE (FIGS. 10D and 11C-11D). Following cleavage of the intein, the scFv is exported through the SEC translocon in an unfolded state, and folding is completed in the periplasm. Thus, it is believed that splitting the signal sequence selects for variants that limit aggregation or degradation occurring after intein-mediated splicing, mediate rapid periplasmic export, or facilitate successful periplasmic folding of the scFv.

High-micromolar and low-nanomolar Kp variants of YibK and Ω-graft performed very differently in pPACE. YibK variant 3.7 and Ω-graft variant 2.8 evolved beneficial mutations in addition to the mutation expected to restore binding affinity to low-nanomolar K_{D} levels. In the case of trastuzumab and the Her2 mimetic peptide H98, however, only modest affinity improvements were evolved from an initially moderate K_{D} (the K_{D} of the trastuzumab IgG-H98 interaction is reported to be 1.4 μM). This outcome may reflect the small surface area of H98 offering fewer opportunities for molecular interaction, or may indicate stringency limitations in the trastuzumab selection. Consistent with these possibilities, further decreasing antigen expression and reducing pIII translation to increase stringency did not lead to enrichment of any new trastuzumab genotypes (FIGS. 15A-15C). Indeed, mutations A34D and Y49S were not present in population 3 after high-stringency evolution, indicating that the increase in expression increases may saturate the limited amount of available H98 in this selection regime, resulting in loss of P_{cadBA} activation. It is believed that stringency of the selection might be further elevated by providing a competitive binder at late stages of the selection, such as a host-encoded copy of the antibody under selection which competes with the phage-encoded form.

H98 has been considered a potential antigen to induce trastuzumab-like antibodies for cancer treatment. It is noted that trastuzumab variants 1.1 and 3.2 showed no change in Her2 affinity as measured by ELISA, indicating that use of H98 as an anticancer mimetic peptide antigen may elicit trastuzumab-like antibodies that retain their affinity for Her2, in agreement with the finding that mice immunized with H98 developed Her2-responsive antibodies. This finding further supports H98 as a candidate antigen for anticancer vaccines. Using a pPACE strategy, trastuzumab or other therapeutic antibodies might also be evolved to bind peptides from growth factor receptors in addition to their native targets to yield bispecific scFvs.

It has been shown that periplasmic PACE can improve both affinity and solubility of Ω-graft and trastuzumab scFvs, and can generate variants of the homodimeric protein YibK with non-covalent and covalent linkages between subunits. Periplasmic PACE represents the first PACE system to select for function in a cell compartment other than the cytoplasm, and the first continuous binding selection in the bacterial periplasmic space. It is believed that this system will be of particular utility in rapid optimization of binding and solubility properties, especially when evolving antibodies to engage antigens that are enriched in disulfide bonds and therefore incompatible with cytoplasmic PACE.

### Materials and Methods

Nuclease-free water (Qiagen) was used for PCR reactions and cloning. PCR reactions were carried out using Phusion U Hot Start DNA polymerase (Thermo Fisher Scientific). Plasmids and SPs were cloned by USER assembly according to manufacturer's instructions. For antibodies and antigens used in this work, synthesized gBlock gene fragments were obtained from Integrated DNA Technologies. E. coli native genes were amplified directly from genomic DNA. Plasmids were cloned and amplified using Turbo (New England BioLabs) cells. Plasmid DNA was amplified for sequencing purposes using the Illustra Templiphi 100 Amplification Kit (GE Healthcare Life Sciences); SP were amplified by PCR using primers AB1793 (5′-TAATGGAAACTTCCTCATGAAAAAGTCTTTAG (SEQ ID NO: 1)) and AB1396(5′-ACAGAGAGAATAACATAAAAACAGGGAAGC (SEQ ID NO: 2)). Phage were sequenced using primers AR007, MM1081, MM1082. TW629 and TW1243. All primer sequences can be found in Table 5. Sanger sequencing was used to confirm all plasmid sequences and to characterize SPs. Phage cloning and phage titer determination was carried out in strain S2208.

Plasmids and phage used in this work can be found in Tables 2-4. Antibiotic (Gold Biotechnology) working concentrations were as follows: carbenicillin 50 μg/mL, spectinomycin 50 μg/mL, chloramphenicol 25 μg/mL, kanamycin 50 μg/mL, tetracycline 10 μg/mL, streptomycin 50 μg/mL.

TABLE 2
Plasmid names, strains, phage and arabinose induction
concentrations used in this work.

| | | | Ara |
|---|---|---|---|
| Strain | Plasmids | Phage | (μM) |
| S2060¹⁰² | PAR26c2 pAR27a/pAR27b/ | | 80 |
| | pAR27c/pAR27d | | |
| S536 | pMM102b2 | SP53a/SP53a2/SP53a3 | |
| S536 | pMM102b2 DP6¹⁰³ | SP53a2 | |
| S536 | pAR26c2 pAR27a/ | | |
| | pAR27c/pMM103g | | |
| S536 | pAR26c2 | | 333.3 |
| | pMM103a/pMM103b/ | | |
| | pMM103g/pMM103i | | |
| S2060 | pAR26c2 pTW200a1/ | | 12.3 |
| | pTW200b1 +/−pMM95a | | |
| S536 | pMM102g1 pMM95g7 DP6 | SP30b1 | |
| S536 | pAR26c2 pMM95h4 | | 333.3 |
| | pMM109d1/ | | |
| | pMM109d2/pMM109d3/ | | |
| | pMM109d4/pMM109d5 | | |
| S1367 | pMM116f pMM114a | 37o5c2.1/40o4c4.2/ | |
| | | 37o5c/40o4c/37o5c-0 | |
| S1367 | pMM116f pMM114a | SP40o4c/SP40o4c4.2 | |
| S1367 | pMM116f pMM114a DP6 | SP37o5c-0 | |
| S1367 | pMM116f pMM114a DP6 | SP40o4c | |
| S1367 | pMM116f pMM114a DP6 | SP37o5c-0/SP40o4c | |
| S536 | pMM96z pMM95h4 | | 333.3 |
| | pMM109d1/d2/d5 | | |
| BL21* | pMM130o1/pMM130o1b | | |
| DE3 | | | |
| S536 | pMM116e2 pMM119d4b | SP37d4/SP40d1/ | |
| | | SP37z4/SP40z1 | |
| S536 | pMM116f pMM119d4b DP6 | SP37d4/SP40d1 | |
| S536 | pMM96z pMM119d4b | | 111.1 |
| | pMM129d1/pMM129d1g/ | | |
| | pMM129d1h | | |
| BL21* | pMM130c1/ | | |
| DE3 | pMM130c1g/pMM130c1h | | |
| S536 | pMM116f pMM119d4b DP6 | SP37d4/SP40d1 | |
| BL21* | pMM130c1/ | | |
| DE3 | pMM130c1g/pMM130c1h | | |
| BL21* | pMM130c1/ | | |
| DE3 | pMM130c1g/pMM130c1h | | |
| S536 | pMM96z pMM119d4b | | |
| | pMM129d1/pMM129d1g/ | | |
| | pMM129d1h/pMM129d3/ | | |
| | pMM129d3g/pMM129 | | |
| | d3h | | |
| S536 | pAR26c2 pMM95a | | 37 |
| | pTW200a1/pMM109a1/ | | |
| | pMM109a2/pMM109a3/ | | |
| | pMM109a4 | | |
| S536 | pMM95g7 pMM116a1 | SP30a1/SP31 | |
| BL21* | pMM130c1/pMM130c1g/ | | |
| DE3 | pMM130c1h | | |
| S1367 | pMM114a pMM116f DP6 | SP37o5c-0 | |
| S1367 | pMM114a pMM116f DP6 | SP40o4c | |
| BL21* | pMM130o1/pMM130o1b | | |
| DE3 | | | |
| BL21* | pMM130c1/pMM130c1g/ | | |
| DE3 | pMM130c1h | | |
| S536 | pMM102g1 pMM95g7 | SP30a1/SP30b1 | |
| | for PACE, pMM102g2 | | |
| | pMM95g7 for PANCE | | |
| S536 | pMM102g1 pMM95g7 DP6 | SP30b1 | |
| S1367 | pMM114a1 | SP37o5C/SP37o5C2.1/ | |
| | pMM116a/pMM116e2/ | SP40o4c/SP40o4c4.2 | |
| | pMM116f/pMM116g | | |
| S536 | pMM96z pMM114a | | 37 |
| | pMM129o1/pMM129o2/ | | |
| | pMM129o3/pMM129o4 | | |
| S1367 | pMM114a1 | SP37o5c | |
| | pMM116a/pMM116e2/ | | |
| | pMM116f/pMM116g | | |
| S536 | pMM116e2 pMM119d4b | SP37d3/SP37z1/ | |
| | | SP37d4/SP37z4 | |
| S536 | pMM116g pMM119d6b | SP37d4/SP40d1 120 h | |
| | DP6 | populations L1, L3 | |
| S536 | pMM102b2 DP6 | SP53a2 | |
| S536 | pAR26c2 pAR27a/pAR27c | | |
| S536 | pAR26c2 pAR27a/ | | |
| | pAR27c/pMM103g | | |
| S2060 | pAR26c2 | SP53a/SP53a2/M13 | |
| S536 | PAR26c2 | SP53a/SP53a2/ | |
| | | SP53a3/M13 | |
| S2060 | pTW161a/pTW161a2 | | |
| | pTW160a/pTW160b/ | | |
| | pTW160c | | |
| BL21* | pMM130c1/pMM130c1g/ | | |
| DE3 | pMM130c1h | | |
| BL21* | pMM130y1/pMM130y2/ | SP30a1 | |
| DE3 | pMM130y3 | | |
| S536 | pMM102b2 pMM95a/ | | 37 |
| | pMM95d | | |
| S1367 | pMM150a1 | | Various |
| | pMM130o1/pMM130o5 | | |
| S1367 | pJC175e⁶ | SP37d4/SP40d1/SP37o5 | |
| | | c/SP40o4c/SP37z1/SP40 | |
| | | z1/SP01a⁹/SP13⁹ | |
| S1367 | pJC175e⁶ | | Various |
| | pMM129d2/pMM129z2 | | |

TABLE 3
Plasmids used in this work. CP: complement plasmid. A complement plasmid takes the
place of an evolving selection phage in plasmid-based assays such as transcription activation assays.

| | Class | | | | | [RBS] |
|---|---|---|---|---|---|---|
| Name | (resistance) | Ori | Prom | [RBS]¹⁰¹ Genes | Prom | Genes |
| DP6 | MP (chlorR) | cloDF-13 | P_{BAD} | dnaQ926, dam, seqA, emrR, ugi, cda1 | P_{c} | araC |
| | | | Pₚₛₚ₋ₜₑₜ | [sd8] gIII | | |
| pAR26c2 | AP1 (carbR) | SC101 | Pₚᵣₒ₃ | [SD8] CadC₁₋₁₅₅-Leu(16)TM⁸-HA4 | P_{CadBA} | [sd8] luxAB |
| pAR27a | CP (specR) | ColE1 | P_{BAD} | [sd8] SS-YibK-SH2 | P_{c} | araC |
| pAR27b | CP (specR) | ColE1 | P_{BAD} | [sd8] YibK-SH2 | P_{c} | araC |
| pAR27c | CP (specR) | ColE1 | P_{BAD} | [sd8] SS-YibK(V139R)-SH2 | P_{c} | araC |
| pAR27d | CP (specR) | ColE1 | P_{BAD} | [sd8] YibK(V139R)-SH2 | P_{c} | araC |
| pMM95a | AP2 (chlorR) | p15A | P_{BAD} | [sd8] SS_{PelB}-GCN4(7P14P)-SH2 | P_{c} | araC |
| pMM95g7 | AP2 (specR) | ColE1 | Pₚᵣₒ₃ | [sd5] CadC₁₋₁₅₅-Leu(16)TM- | Pₚᵣₒ₃ | [SD8] SS₁₋₈-NpuN |
| | | | | GCN4(7P14P) | | |
| pMM95h4 | AP2 (chlorR) | p15A | P_{BAD} | [sd5] CadC₁₋₁₅₅-Leu(16)TM- | Pₚᵣₒ₃ | [SD8] SS₁₋₈-NpuN |
| | | | | GCN4(7P14P) | | |
| | | | Pc | araC | | |
| pMM96z | AP1 (carbR) | SC101 | P_{CadBA} | [sd8] luxAB | | |
| pMM102b2 | AP1 (carbR) | SC101 | Pₚᵣₒ₁ | [sd8] CadC₁₋₁₅₅-Leu(16)TM-HA4 | P_{CadBA} | [sd8] gIII |
| pMM102g1 | AP1 (carbR) | SC101 | Pₚᵣₒ₁ | [sd6] CadC₁₋₁₅₅-Leu(16)TM-HA4 | P_{CadBA} | [sd8] gIII |
| pMM102g2 | AP1 (carbR) | SC101 | Pₚᵣₒ₁ | [sd5] CadC₁₋₁₅₅-Leu(16)TM-HA4 | P_{CadBA} | [sd8] gIII |
| pMM102g7 | AP1 (carbR) | SC101 | Pₚᵣₒ₁ | [sd4U] CadC₁₋₁₅₅-Leu(16)TM-HA4 | P_{CadBA} | [sd5] gIII |
| pMM103a | CP (specR) | ColE1 | P_{BAD} | [sd8] SS-YibK-SH2 | P_{c} | araC |
| pMM103b | CP (specR) | ColE1 | P_{BAD} | [sd8] SS-YibK(V139R)-SH2 | P_{c} | araC |
| pMM103g | CP (specR) | ColE1 | P_{BAD} | [sd8] SS-YibK(V139R R146C)-SH2 | P_{c} | araC |
| pMM103i | CP (specR) | ColE1 | P_{BAD} | [sd8] SS-YibK(V139R A138D)-SH2 | P_{c} | araC |
| pMM109a1 | CP (specR) | ColE1 | Pₚᵣₒ₁ | [sd5] SS_{8-CNF-9}-Ω-graft-SH2 | | |
| pMM109a2 | CP (specR) | ColE1 | Pₚᵣₒ₁ | [sd5] SS_{9-CNF-10}-Ω-graft-SH2 | | |
| pMM109a3 | CP (specR) | ColE1 | Pₚᵣₒ₁ | [sd5] SS_{10-CNF-11}-Ω-graft-SH2 | | |
| pMM109a4 | CP (specR) | ColE1 | Pₚᵣₒ₁ | [sd5] SS_{11-CNF-12}-Ω-graft-SH2 | | |
| pMM109d1 | CP (specR) | ColE1 | Pₚᵣₒ₃ | [sd5] NpuC-SS₉₋₂₁-Ω-graft-SH2 | | |
| pMM109d2 | CP (specR) | ColE1 | Pₚᵣₒ₃ | [sd5] NpuC-SS₉₋₂₀-Ω-graft(L231F | | |
| | | | | 232A)-SH2 | | |
| pMM109d3 | CP (specR) | ColE1 | Pₚᵣₒ₃ | [sd5] NpuC-SS₉₋₂₀-Ω-graft(L224S | | |
| | | | | L231F F232A)-SH2 | | |
| pMM109d4 | CP (specR) | ColE1 | Pₚᵣₒ₃ | [sd5] NpuC-SS₉₋₂₀-Ω-graft(R1C | | |
| | | | | L231F F232A)-SH2 | | |
| pMM109d5 | CP (specR) | ColE1 | Pₚᵣₒ₃ | [sd5] NpuC-SS₉₋₂₀-Ω-graft(G119C | | |
| | | | | L231F F232A)-SH2 | | |
| pMM114a | AP2 (specR) | ColE1 | Pₚᵣₒ₃ | [sd5] CadC₁₋₁₅₅-Leu(16)TM- | Pₚᵣₒ₃ | [SD8] SS₁₋₈-NpuN |
| | | | | GCN4(7P14P) | | |
| pMM116a | AP1 (carbR) | SC101 | P_{cadBA} | [sd8] gIII | | |
| pMM116e2 | AP1 (carbR) | SC101 | P_{cadBA} | [sd6] gIII, luxAB | | |
| pMM116f | AP1 (carbR) | SC101 | P_{cadBA} | [sd2] gIII | | |
| pMM116g | AP1 (carbR) | SC101 | P_{cadBA} | [sd2G] gIII | | |
| pMM119d4b | AP2 (specR) | ColE1 | Pₚᵣₒ₃ | [sd5] CadC₁₋₁₅₅-Leu(16) TM-H98 | Pₚᵣₒ₃ | [SD8] SS₁₋₈-NpuN |
| pMM119d6b | AP2 (specR) | ColE1 | Pₚᵣₒ₁ | [sd5] CadC₁₋₁₅₅-Leu(16) TM-H98 | Pₚᵣₒ₃ | [SD8] SS₁₋₈-NpuN |
| pMM129d1 | CP (chlorR) | p15A | P_{BAD} | [sd8] SS-trastuzumab-YibK | P_{c} | araC |
| pMM129d1g | CP (chlorR) | p15A | P_{BAD} | [sd8] SS-trastuzumab(H91Y)-YibK | P_{c} | araC |
| pMM129d1h | CP (chlorR) | p15A | P_{BAD} | [sd8] SS-trastuzumab(A34D Y49S)- | P_{c} | araC |
| | | | | YibK | | |
| pMM129d3 | CP (chlorR) | p15A | P_{BAD} | [sd8] SS-trastuzumab(C23S C88S | P_{c} | araC |
| | | | | C150S C224S)-YibK | | |
| pMM129d3g | CP (chlorR) | p15A | P_{BAD} | [sd8] SS-trastuzumab(C23S C88S | P_{c} | araC |
| | | | | H91Y C150S C224S)-YibK | | |
| pMM129d3h | CP (chlorR) | p15A | P_{BAD} | [sd8] SS-trastuzumab(C23S A34D | P_{c} | araC |
| | | | | Y49S C88S C150S C224S)-YibK | | |
| pMM129o1 | CP (chlorR) | p15A | P_{BAD} | [sd8] SS-Ω-graft-YibK | P_{c} | araC |
| pMM129o2 | CP (chlorR) | p15A | P_{BAD} | [sd8] SS-Ω-graft(L231F)-YibK | P_{c} | araC |
| pMM129o3 | CP (chlorR) | p15A | P_{BAD} | [sd8] SS-Ω-graft(F232A)-YibK | P_{c} | araC |
| pMM129o4 | CP (chlorR) | p15A | P_{BAD} | [sd8] SS-Ω-graft(231F 232A)-YibK | P_{c} | araC |
| pMM130c1 | EP (kanR) | pBR322 | P_{T7lac} | SS-trastuzumab-6XHis | | |
| pMM130c1g | EP (kanR) | pBR323 | P_{T7lac} | SS-trastuzumab(91Y)-6XHis | | |
| pMM130c1h | EP (kanR) | pBR323 | P_{T7lac} | SS-trastuzumab(34D 49S)-6XHis | | |
| pMM130o1 | EP (kanR) | pBR322 | P_{T7lac} | SS-Ω-graft-6XHis | | |
| pMM130o1b | EP (kanR) | pBR323 | P_{T7lac} | SS-Ω-graft(224S)-6XHis | | |
| pTW161a | CP (specR) | ColE1 | P_{BAD} | [sd8] CadC₁₋₁₅₅-Leu(16)TM-GCN4 | P_{c} | araC |
| pTW161a2 | CP (specR) | ColE1 | P_{BAD} | [sd8] CadC₁₋₁₅₅-Leu(16)TM- | P_{c} | araC |
| | | | | GCN4(7P14P) | | |
| pTW160a | AP1 (carbR) | SC101 | P_{cadBA} | luxAB | | |
| pTW160b | AP1 (carbR) | SC101 | P_{cadBA (-427-+1)} | luxAB | | |
| pTW160ac | AP1 (carbR) | SC101 | P_{cadBA (-288-+1)} | luxAB | | |
| pTW200a1 | AP2 (specR) | ColE1 | Pₚᵣₒ₁ | [sd5] SS-Ω-graft-SH2 | | |
| pTW200b1 | AP2 (specR) | ColE2 | Pₚᵣₒ₁ | [sd5] SS-Ω-graft(L231F F232A)-SH2 | | |
| pMM95D | AP2 (chlorR) | p15A | P_{BAD} | [sd8] SS_{PelB}-GCN4(7P14P)-SH2 | Pₚᵣₒ₃ | [SD8] SS₁₋₈-NpuN |
| pMM129d2 | CP (chlorR) | p15A | P_{BAD} | [sd8] SS-trastuzumab-GCN4 | P_{c} | araC |
| pMM129z2 | CP (chlorR) | p15A | P_{BAD} | [sd8] SS_{PelB}-GCN4 | P_{c} | araC |
| pMM130o5 | EP (kanR) | pBR323 | P_{T7lac} | NpuC-SS9-20-Ω-graft-6XHis | | |
| pMM130y1 | EP (kanR) | pBR323 | P_{T7lac} | SS-YibK-6XHis | | |
| pMM130y2 | EP (kanR) | pBR323 | P_{T7lac} | SS-YibK(V139R)-6XHis | | |
| pMM130y3 | EP (kanR) | pBR323 | P_{T7lac} | SS-YibK(V139R R146C)-6XHis | | |
| pMM150a1 | CP (specR) | ColE1 | P_{BAD} | [sd5] SS₁₋₈-NpuN | P_{c} | araC |

TABLE 4
Selection phage used in this work.

| Name | Class | Ori | Prom | [RBS]¹⁰⁰ Genes |
|---|---|---|---|---|
| M13 | Ref. | M13 f1 | P_{glll} | glll |
| SP30a1 | Selection | M13 f1 | P_{glll} | [sd8] NpuC-SP₉₋₂₀- |
| | phage | | | Ω-graft-SH2 |
| SP30b1 | SP | M13 f1 | P_{glll} | [sd8] NpuC-SP₉₋₂₀-Ω- |
| | | | | graft(L231F F232A)-SH2 |
| SP31 | SP | M13 f1 | P_{glll} | [sd8] SP₉₋₂₀-Ω-graft-SH2 |
| SP35a1 | SP | M13 f1 | P_{glll} | [sd8] NpuC-SP₉₋₂₀-Ω- |
| | | | | graft(G119C) |
| SP35a2 | SP | M13 f1 | P_{glll} | [sd8] NpuC-SP₉₋₂₀-Ω- |
| | | | | graft(G119C L231F |
| | | | | F232A) |
| SP37d3 | SP | M13 f1 | P_{glll} | [sd8] SP-trastuzumab-YibK |
| SP37d4 | SP | M13 f1 | P_{glll} | [sd8] SP-trastuzumab-GCN4 |
| SP37o5c | SP | M13 f1 | P_{glll} | [sd8] SP-Ω- |
| | | | | graft(R1C L231F F232A) |
| SP37o5c-0 | SP | M13 f1 | P_{glll} | [sd8] SP-Ω-graft(R1C |
| | | | | W100* L231F |
| | | | | F232A) |
| SP37o5c2.1 | SP | M13 f1 | P_{glll} | [sd8] SP-Ω-graft(R1C F232A) |
| SP37z1 | SP | M13 f1 | P_{glll} | [sd8] SP-YibK |
| SP37z4 | SP | M13 f1 | P_{glll} | [sd8] SP-GCN4 |
| SP40d1 | SP | M13 f1 | P_{glll} | [sd8] NpuC- |
| | | | | SP₉₋₂₀-trastuzumab-GCN4 |
| SP40o4c | SP | M13 f1 | P_{glll} | [sd8] NpuC-SP₉₋₂₀-Ω- |
| | | | | graft(R1C L231F |
| | | | | F232A) |
| SP40o4c4.2 | SP | M13 f1 | P_{glll} | [sd8] NpuC-SP₉₋₂₀- |
| | | | | Ω-graft(R1C L232A) |
| SP40z1 | SP | M13 f1 | P_{glll} | [sd8] NpuC-SP₉₋₂₀-GCN4 |
| SP53a | SP | M13 f1 | P_{glll} | [sd8] SP-YibK-SH2 |
| SP53a2 | SP | M13 f1 | P_{glll} | [sd8] SP-YibK(V139R)-SH2 |
| SP53a3 | SP | M13 f1 | P_{glll} | [sd8] SP-SH2 |
| SP01a⁹ | SP | M13 f1 | P_{glll} | [RpoZ] MBP-T7N |
| SP13⁹ | SP | M13 f1 | P_{glll} | [RpoZ] KanR |

TABLE 5
Primers used in this work.

| | | SEQ |
|---|---|---|
| | | ID NO: |
| AB1793 | (5′-TAATGGAAACTTCCTCATGAAAAAGTCTTTAG) | 1 |
| AB1396 | 5′-ACAGAGAGAATAACATAAAAACAGGGAAGC) | 2 |
| AR007 | (5′-ATGTTGAAAATCTCCTTCUAGATTA) | 38 |
| MM557 | (5′- | 3 |
| | TGTGGCAATTATCATTGCATCATTCCCTTTTCGAATGAGTTTCTA | |
| | TTATGTGTAGGCTGGAGCTGCTTCG) | |
| MM558 | 5′-AAAATAACGTCTTGCATTCACC) | 5 |
| MM559 | 5′- | 4 |
| | TGGCAAGCCACTTCCCTTGTACGAGCTAATTATTTTTTGCTTTCT | |
| | TCTTTATTCCGGGGATCCGTCGACC | |
| MM560 | (5′-TTCATGTGTTCTCCTTATGAGC) | 6 |
| MM1081 | (5′-AGGTGGGGGTUCAGGC) | 39 |
| MM1082 | (5′-AACCCCCACCUCCGGAG) | 40 |
| TW629 | (5′-GACTCCCTGCAAGCCTCAG) | 41 |
| TW1243 | (5′-CGAACCAGAAAAGAACCATTAATG) | 34 |

TABLE 6
Properties of trastuzumab scFv and evolved
variants determined by MST analysis.
Values reflect mean and s.d. of three technical
replicates in MST (FIGS. 9A-9B).

| | EC₅₀ (μM) | K_{D} (μM) |
|---|---|---|
| TR | 4.3 ± 1.6 | 44.9 ± 8.7 |
| 1.1 | 1.4 ± 1.1 | 20.5 ± 1.9 |
| 3.2 | 1.3 ± 1.1 | 18.8 ± 0.7 |

### Preparation and Transformation of Competent Cells

To prepare chemically competent cells of strains S536, S1367 and S2208, overnight cultures were grown from single colonies and diluted 500-fold into 10 mL of 2×YT media (United States Biologicals) supplemented with appropriate antibiotics. Cells were grown at 37° C. with 230 RPM shaking to OD₆₀₀=0.4-0.6 and pelleted by centrifugation at 4000 g for 10 minutes at 4° C. The cell pellet was then resuspended in 500 μL TSS (LB media supplemented with 2.5% v/v DMSO, 5% w/v PEG 3350, and 10 mM MgCl₂). For transformations, 50 μL of competent cells were added to 1 μL plasmid in 50 μL pre-chilled KCM (100 mM KCl, 30 mM CaCl₂), and 50 mM MgCl₂ in H₂O), incubated on ice for 15 minutes, heat shocked at 42° C. for 90 seconds and incubated on ice 2 minutes prior to recovery.

To prepare electrocompetent cells of strains S1021, S536, S1367, single colonies or glycerol stocks were grown up overnight and diluted 500-fold in 2×YT plus appropriate antibiotics. 10 mL of cells at OD₆₀₀ 0.3-0.4 were pelleted by centrifugation at 4000 g for 10 minutes at 4° C. The cell pellet was resuspended in 1 mL ice-cold 10% glycerol and washed 3× with 1 mL ice-cold glycerol, pelleting at 10,000 g for 1 minute at 4° C. between washes and maintaining cells on ice between spins. The pellet was resuspended in 500 μL ice-cold 10% glycerol and the resulting mixture used fresh or else stored at −80° C. For transformation, 1 μL each of up to three plasmids was added directly to 50 μL of electrocompetent cells prior to electroporation in pre-chilled cuvettes (Bio-Rad).

Cells were recovered for 1 hr at 37° C. with shaking at 230 RPM in 1 mL of SOC media (New England BioLabs) and streaked on 2×YT media+1.5% agar (United States Biologicals) plates containing the appropriate antibiotics before incubation at 37° C. for 12-18 h.

E. coli Strains

All luminescence assays and evolution experiments were carried out in E. coli strains S536 and S1367. These strains were engineered from PACE strains S1030 and S2060 respectively, using Lambda Red recombineering to replace the E. coli native CadCBA operon with a kanamycin resistance cassette. Chemically competent host cells of strain S1021 were transformed with plasmid pKD119 as described above. Primers MM557 (5′-TGTGGCAATTATCATTGCATCATTCCCTTTTCGAATGAGTTTCTATTATGTGTAGGCT GGAGCTGCTTCG (SEQ ID NO: 3)) and MM559 (5′-TGGCAAGCCACTTCCCTTGTACGAGCTAATTATTTTTTGCTTTCTTCTTTATTCCGGG GATCCGTCGACC (SEQ ID NO: 4)), with 5′ homology to regions of the genome flanking the cadCBA operon, were used to amplify the kanamycin resistance cassette from plasmid pKD13. The PCR product was gel-purified and transformed into 500 μL S1021+pKD119 cells by electroporation and recovered overnight at 37° C. with shaking at 230 RPM in 4 mL SOC, then plated on 2×YT+1.5% agar+kanamycin and incubated at 37° C. for 16 hours.

Insertion of the kanamycin resistance cassette was verified by colony PCR using primers MM558 (5′-AAAATAACGTCTTGCATTCACC (SEQ ID NO: 5)) and MM560 (5′-TTCATGTGTTCTCCTTATGAGC (SEQ ID NO: 6)). Successful colonies were inoculated into 2×YT+kanamycin and grown up at 37° C. for 5 hours before plating in parallel on 2×YT+1.5% agar+kanamycin or tetracycline to verify successful curing of pKD119. Successful cultures were incubated in 2×YT+kanamycin for 2 hrs at 37° C. with the addition of 1 μL of F-plasmid donor culture, S103030 or S206034, and streaked on 2×YT+1.5% agar+kanamycin, tetracycline and streptomycin. Since loss of the cadCBA operon is associated with a slight fitness cost, ΔcadCBA cells were maintained with kanamycin throughout subsequent work to safeguard against contamination by strains lacking the ΔcadCBA deletion.

### Luciferase Transcriptional Activation Assay

S536 and S1367 cells were transformed with APs and CPs as indicated in Table 3. Freshly saturated cultures of single colonies grown in Davis Rich Media (DRM) plus maintenance antibiotics were diluted 500-fold into DRM media with maintenance antibiotics in a 96-well deep well plate (Axygen) and induced with indicated concentrations of arabinose (Gold Biotechnology) before incubation for 2 h at 37° C. with shaking at 230 RPM. 150 μL of cells per well were then transferred to a 96-well black-walled clear-bottomed plate with a transparent lid (Costar). 600 nm absorbance and luminescence were read at 15-minute intervals over an 8-hour kinetic cycle with shaking at 230 RPM between reads using a Tecan Spark multimode microplate reader (Tecan). Single read data were taken at peak luminescence value (4-5 hours post-induction). OD₆₀₀-normalized luminescence values were determined by dividing raw luminescence by background-subtracted (DRM only) 600 nm absorbance.

For phage-induced luciferase time course assay, S536 and S2060 cells were transformed with Aps and diluted in DRM as described above. Cells were grown to an OD₆₀₀ of 0.4 and were inoculated with selection phage at an initial titer of 5×10⁴ pfu/mL. 150 μL of cells per well were immediately transferred to a plate for luminescence and optical density reading in a kinetic cycle as described above.

### Phage Propagation Assay

S536 and S1367 cells were transformed with the AP(s) of interest as described above. Overnight cultures of single colonies grown in 2×YT media supplemented with maintenance antibiotics were diluted 1000-fold into DRM media with maintenance antibiotics and grown at 37° C. with shaking at 230 RPM to OD₆₀₀ 0.4 exactly. Cells were infected with SP at an initial titer of 5×10⁴ pfu/mL⁻¹. Cells were incubated 16-18 hours at 37° C. with shaking at 230 RPM, then centrifuged at 10,000 g for 2 minutes and the supernatant stored at 4° C.

### Plaque Assay

Saturated cultures of single colonies of strain S2208 grown in 2×YT media plus maintenance antibiotics were diluted 1000-fold into fresh 2×YT media with maintenance antibiotics and grown at 37° C. with shaking at 230 RPM to OD₆₀₀ ˜0.8 before use. SP were serially diluted 100-fold (4 dilutions total) in H₂O. 10 μL of phage dilution was added to 150 μL of cells and immediately mixed with 1 mL of liquid (55° C.) top agar (2×YT media+0.6% agar) supplemented with 2% Bluo-gal (Gold Biotechnology). The mixture was then immediately pipetted onto one quadrant of a quartered Petri dish containing 2 mL of solidified bottom agar (2×YT media+1.5% agar, no antibiotics) and allowed to solidify. Plates were incubated at 37° C. for 16-18 h. Titers were rounded to one significant figure prior to calculating ratios.

### Phage-Assisted Continuous and Non-Continuous Evolution

Cell preparation, PANCE and PACE was carried out in DRM.

Chemically competent S536 or S1367 cells were transformed with AP(s) and DP6, plated on 2×YT media+1.5% agar supplemented with 10 mM glucose (to suppress induction of mutagenesis from the P_{BAD} promoter) and maintenance antibiotics, and grown at 37° C. for 16 hours. Colonies were picked into 500 μL DRM in a 96-well deep-well plate, and serially diluted 10-fold twelve times in DRM. Typically, eight colonies were selected. The plate was sealed with porous film and colonies allowed to grow at 37° C. with shaking at 230 RPM for 16-18 hours.

For PACE, dilutions with OD₆₀₀ ˜0.4-0.8 were then used to inoculate an 80 mL DRM chemostat. The chemostat was continuously diluted with fresh DRM at a rate of ˜1.5 chemostat volumes/h, maintaining a volume of 60-80 mL and an OD₆₀₀ value between 0.8-1.0, as previously described.

Lagoons were continuously diluted from the chemostat culture at 1 lagoon volume/hour and were induced with 10 mM arabinose+/−50 ng/mL aTc as indicated, for at least 2 hours prior to infection with SP. For novel PACE campaigns, SP were plaqued as described above and purified from single plaques by growing up ˜8 hours in fresh 2×YT media with maintenance antibiotics at 37° C. with shaking at 230 RPM. For continuations of previous PACE runs at increased stringency, 20 μL of lagoon samples from previous PACE endpoints were added to 2 mL of S2208 cells in mid-log growth phase and grown for ˜4 hours in 2×YT media plus maintenance antibiotics at 37° C. with shaking at 230 RPM. All selection phage cultures were centrifuged at 10,000 g for 2 minutes and passed through a 0.22-μm PVDF Ultrafree centrifugal filter (Millipore) prior to use in PACE.

Lagoons were infected with purified SP at a starting titer of 10-106 pfu/mL and maintained at a volume of 15 mL through constant inflow of chemostat material and outflow of media waste at a rate of 0.5-3 lagoon volumes per hour. Arabinose and aTc concentrations within lagoons were maintained through constant inflow. 500-μL samples were taken at indicated times from lagoon waste lines. Samples were centrifuged at 10,000 g for 2 minutes, and the supernatant was passed through a 0.22-μm PVDF Ultrafree centrifugal filter (Millipore) and stored at 4° C.

Selection phage titers were determined by plaque assays using S2208 cells. Four or eight single plaques were PCR amplified as described above to characterize lagoon phage.

For PANCE, host strain dilutions with OD₆₀₀ ˜0.4-0.8 were further diluted to 50 mL in DRM plus appropriate antibiotics and grown up to OD₆₀₀ ˜0.4. 1 mL of cells were added to each well of a deep-well plate, allocating one well per replicate. Wells were induced with 10 mM arabinose if mutagenesis/drift plasmid was present and were inoculated with phage at 107 pfu/mL unless otherwise indicated. Plates were grown up 16 hours at 37° C. with shaking at 230 RPM. Plaques were amplified for characterization as described above. For restriction-enzyme-mediated phage characterization, 400 ng PCR-amplified phage DNA was cleaved with 0.4 μL HinfI (New England Biolabs) according to manufacturer's instructions.

### Small-Scale Protein Expression

BL21 DE3 cells (New England BioLabs) were transformed with expression plasmids (EPs) according to the manufacturer's protocol. Single colonies were grown up overnight in 2×YT media plus maintenance antibiotics were diluted 1000-fold into fresh 2×YT media (2 mL) with maintenance antibiotics and grown at 37° C. with shaking at 230 RPM to OD₆₀₀ 0.4. Cells were induced with 0.1 mM isopropyl-β-D-thiogalactoside (IPTG; Gold Biotechnology) or other indicated concentration and grown for a further 4 hours at 37° C. with shaking at 230 RPM. 2 OD₆₀₀ units of culture were isolated by centrifugation at 8000 g for 2 minutes. The resulting pellet was resuspended in 150 μL B-per reagent (Thermo Fisher Scientific) supplemented with protease inhibitor cocktail (Roche) and incubated at 25° C. for 15 minutes before centrifugation at 16,000 g for 2 minutes. The supernatant was collected as the soluble fraction. The pellet was resuspended in an additional 150 μL B-per reagent to obtain the insoluble fraction. To 37.5 μL of each fraction was added 12.5 μL 4× NuPage LDS sample buffer (Thermo Fisher Scientific). Fractions were vortexed and incubated at 95° C. for 10 minutes. 12 μL (soluble fraction) or 5 μL (insoluble fraction) was loaded per well of a Bolt 4-12% Bis-Tris Plus (Thermo Fisher Scientific) pre-cast gel. 5 μL of Precision Plus Protein Dual Color Standard (Bio-Rad) was used as a reference. Samples were separated by electrophoresis at 200 V for 30 minutes in Bolt MES SDS running buffer (Thermo Fisher Scientific). Gels were stained with InstantBlue reagent (Expedcon) for −16 hours and destained for 1 hour in water before imaging with a G:Box Chemi XRQ (Syngene).

### Periplasmic Extraction

Periplasmic extraction was carried out. Briefly, 100 mL of cells at OD₆₀₀=˜1 were pelleted by centrifugation at 3000 g, drained, and carefully resuspended in 1 mL TSE buffer (200 mM Tris-HCl pH 8.0, 500 mM sucrose, 1 mM EDTA) plus protease inhibitor cocktail (Roche). Cell pellets were incubated on ice for 30 minutes and supernatant (periplasmic extract) was separated from cell pellet (spheroplasts) by centrifugation at 16,000 g for 30 minutes at 4C. Cell pellet was lysed in B-PER as described above. Samples were analyzed by SDS-PAGE and Western blot.

### Western Blot Analysis

Following SDS-PAGE, proteins were transferred to a PVDF membrane using an iBlot 2 Gel Transfer Device (Thermo Fisher Scientific) according to the manufacturer's protocol. The membrane was blocked in SuperBlock Blocking Buffer (Thermo Fisher Scientific) for 1 hour at room temperature, then incubated overnight at 4° C. in SuperBlock Blocking Buffer (Thermo Fisher Scientific) plus one or more of the following, as indicated: mouse anti-6×His (abcam ab18184; 1:2000 dilution), mouse anti-c-ABL (Sigma-Aldrich A5844; 1:2000 dilution), mouse anti-MBP (abcam ab65, 1:5000 dilution) and rabbit anti-GroEL (Sigma-Aldrich G6532; 1:20,000 dilution). If both primary and loading control antibodies were mouse-derived, as in FIG. 8G, membrane was cut according to expected MW of target and membrane halves were incubated separately in primary antibodies, as indicated. The membrane was washed 3× with TBST (TBS+0.5% Tween-20) for 10 minutes each at room temperature, then incubated with IRDye-labeled secondary antibodies goat anti-mouse 680RD (LI-COR 926-68070) and donkey anti-rabbit 800CW (LI-COR 926-32213) diluted 1:5000 for 1 hour at 25° C. The membrane was washed 3× with TBS as before. Imaging was performed using the Odyssey Imaging System (LI-COR).

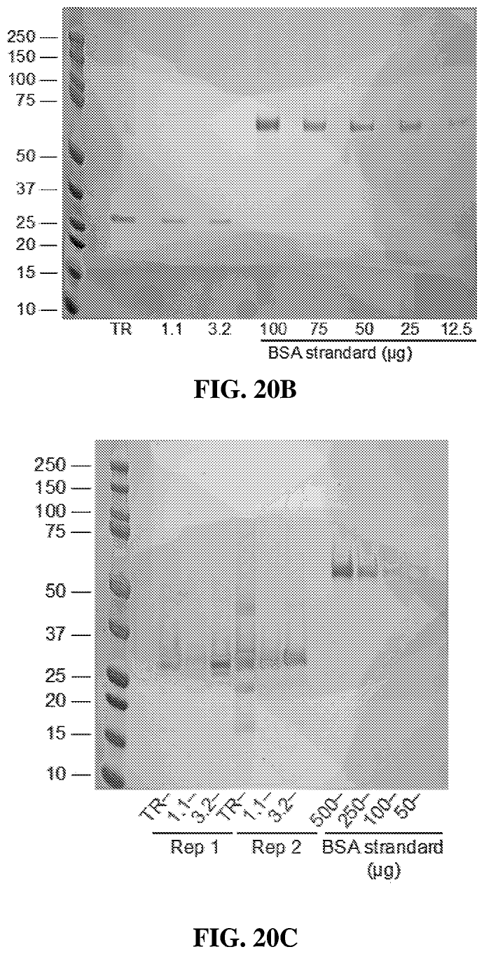

Band densities were quantified using ImageJ and normalized to reference bands to control for loading. Uncropped blot images can be found in FIGS. 10A-10E, FIGS. 11A-11G, and FIGS. 20A-20C.

### Large-Scale Protein Expression and Purification

BL21 DE3 cells transformed with EPs of interest were grown in LB or 2×YT media containing maintenance antibiotics overnight from single colonies. Cultures were diluted 1000-fold into fresh 2×YT media (1 L) with appropriate antibiotics and grown up at 37° C. with shaking at 230 RPM to OD₆₀₀ ˜0.4-0.5. Cells were induced with 50 uM IPTG and grown for a further 16-18 hour at 16° C. with shaking at 200 RPM. Cells were isolated by centrifugation at 8000 g for 10 minutes and washed 1× with 20 mL TBS (20 mM Tris-Cl, 500 mM NaCl, pH 7.5).

The resulting pellet was resuspended in 12 mL B-per reagent supplemented with EDTA-free protease inhibitor cocktail (Roche) and incubated on ice for 30 minutes with regular vortexing, before centrifugation at 16,000 g for 18 minutes. The supernatant was decanted into a 50 mL conical tube and incubated with 1 mL of TALON Cobalt (Clontech) resin at 4° C. with constant agitation for 2 h, after which the resin was isolated by centrifugation at 500 g for 5 minutes. The supernatant was decanted, and the resin resuspended in 4 mL binding buffer (50 mM NaH₂PO₄, 300 mM NaCl, 20 mM imidazole, pH 7.8) and transferred to a column. The resin was washed 4× with 4 mL binding buffer before protein was eluted with 2×1 mL of binding buffer containing increasing concentrations of imidazole (50-300 mM in 50 mM increments). The fractions were analyzed by SDS-PAGE. Combined pure fractions were buffer-exchanged with TBS and concentrated using an Amicon Ultra-15 centrifugal filter unit (10,000 molecular weight cutoff; Millipore), then stored at 4° C. for up to one week or else snap-frozen in liquid nitrogen for-80C storage. Total protein was quantified using a BCA protein assay kit (Pierce) using BSA standards (Bio-Rad). Quantification of specific bands, where necessary, was carried out by gel densitometry using ImageJ software with comparison to reference lanes loaded with known quantities of BSA (Bio-Rad).

### ELISA

Pre-blocked high-capacity streptavidin-coated 96-well clear plates (Pierce) were washed 3× with 200 ul/well TBST and incubated overnight at 4C with purified biotin-tagged protein (Her2, TGFB1, AcroBiosystems; H98 peptide, biotin-GGGGSLLGPYELWELSH (SEQ ID NO: 7), GenScript Custom Peptide) diluted as indicated in TBS. After overnight incubation, wells were washed 3× with 200 ul/well TBST and incubated at room temperature for 2 hour with 25 ug/mL purified antibody fragments in TBS, 50 uL per well. Wells were washed 3× with 200 ul/well TBST and incubated for a further 45 minutes with protein a-HRP (Thermo Fisher Scientific 101023, 1:2000 dilution) in TBS. Finally, wells were washed 4× with 200 ul/well TBST, then developed with 50 μL/well 1-Step Ultra TMB-ELISA Substrate (Thermo Fisher Scientific) for 90 seconds. Quenching was carried out with 50 μL/well 2 M H₂SO₄ and 450 nm absorbance was read using a Tecan Spark multimode microplate reader. Values were normalized by subtracting the mean value at 0 nM antigen for each variant and dividing all values by the maximum mean value of the unmutated TR control. EC₅₀ values were calculated using a sigmoidal 4-point linear regression in Prism 8.

### Micro-Scale Thermophoresis

MST was carried out using the Monolith NT.115 system (Nanotemper) according to the manufacturer's instructions. H98 peptide (GenScript) was resuspended in DMSO and diluted in TBS-T to a final concentration of 6.25% DMSO. Trastuzumab and variant scFvs were diluted in TBS-T to a final concentration of 5 nM and fluorophore-tagged with cy3-conjugated anti-6λH antibody (Rockland Antibodies & Assays) at a 1:1 molar ratio. Reads were carried out using Monolith.NT automated capillary chips (Nanotemper). Data was analyzed with built-in MO.Control and MO.Affinity Analysis software.

### Growth Time-Course Assay

For phage-based growth time-course assays, S1367 cells were transformed with permissive accessory plasmid pJC175e. Freshly saturated cultures of single colonies grown in DRM media plus maintenance antibiotics were diluted 1000-fold into DRM media with maintenance antibiotics until OD₆₀₀ ˜0.1 was reached. Biological replicates were infected with phage at indicated initial titers, and 150 μL of cells per well were immediately transferred to a 96-well black-walled clear-bottomed plate with a transparent lid (Costar). 600 nm absorbance and luminescence were read at 10-minute intervals over a 9-hour kinetic cycle with shaking at 230 RPM between reads using a Tecan Spark multimode microplate reader (Tecan).

For plasmid-based growth time-course assays, S1367 cells were transformed with pJC175e and CPs as indicated in Table 3. Freshly saturated cultures of single colonies grown in DRM media plus maintenance antibiotics were diluted 500-fold into DRM media with maintenance antibiotics in a 96-well deep well plate (Axygen) and induced with indicated concentrations of arabinose (Gold Biotechnology) before incubation for 2 hours at 37° C. with shaking at 230 RPM. 150 μL of cells per well were then transferred to a 96-well black-walled clear-bottomed plate with a transparent lid (Costar). 600 nm absorbance and luminescence were read at 10-minute intervals over a 9-hour kinetic cycle with shaking at 230 RPM between reads using a Tecan Spark multimode microplate reader (Tecan).

### Protein Melt Temperature Assay

Melt temperatures were determined using the Protein Thermal Shift Dye Kit (Life Technologies) according to manufacturer's protocols. A CFX96 Real-Time PCR Detection System (Bio-Rad) was used to monitor fluorescence.

### Example 2

### Evolution of Periplasmic PACE to Develop Therapeutic Antibodies

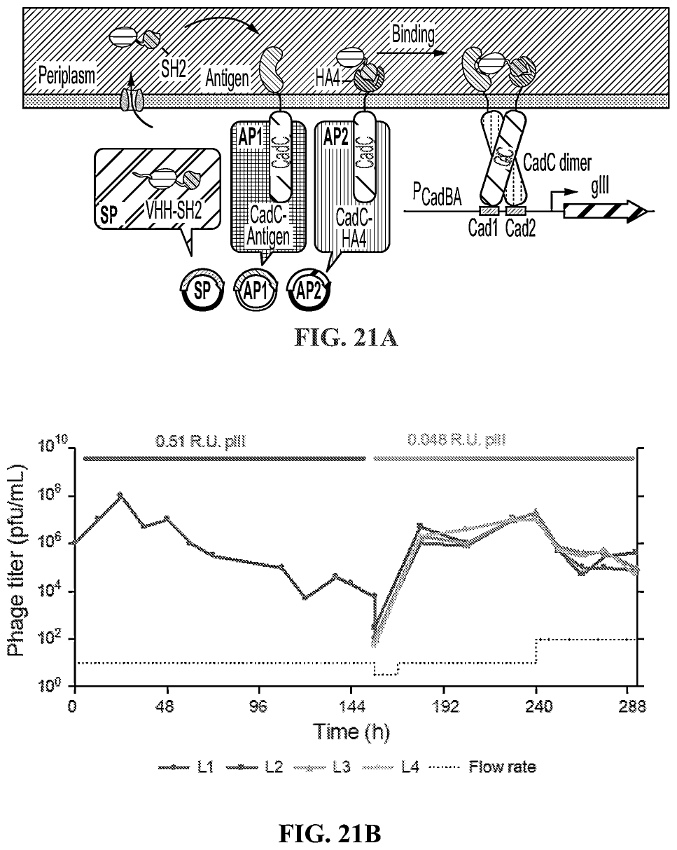

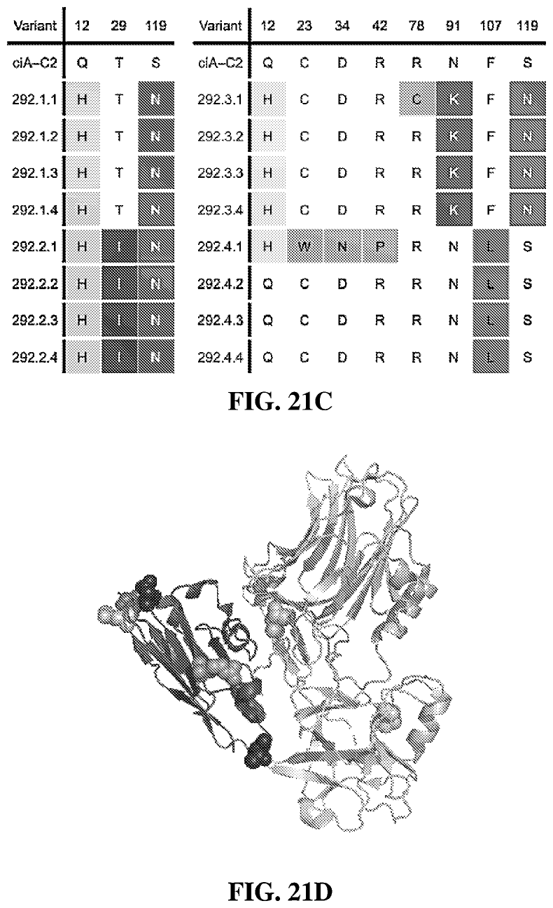

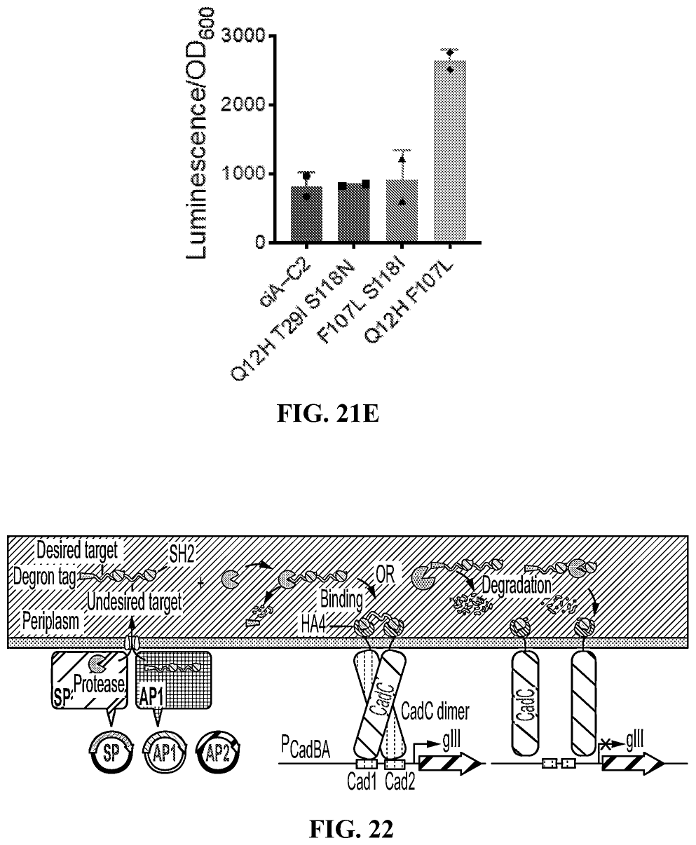

In order to explore the selection topologies compatible more deeply with periplasmic selection, evolution of a monomeric immune protein, a camelid single-domain antibody (also called a VHH) in an asymmetric format (FIGS. 21A-21E) was investigated. A target of prokaryotic origin, the receptor-binding domain of Botulinum neurotoxin (BoNT) from Clostridium botulinum, was selected. BoNT neurotoxins comprise a heavy chain including a receptor-binding domain (RBD) which binds receptors to induce internalization into neuronal cells, and a light chain consisting of a metalloprotease, which is released from the heavy chain by the reduction of an intra-chain disulfide. The liberated light chain goes on to cleave SNARE proteins involved in vesicular trafficking.

Currently, antibodies represent the only treatment for botulism, the potentially fatal condition of flaccid paralysis brought on by intoxication with BoNT. FDA-approved treatment modalities consist of costly monoclonal antibodies or polyclonal antibody mixtures prone to side effects. The most potent and fatal serotype, BoNT/A, can be neutralized by a VHH-derived antitoxin, ciA-C2, which binds the receptor-binding domain (RBD) of the toxin and directly interferes with binding of the toxin to its receptor.

However, ciA-C2 fails to bind a related serotype, BoNT/H, despite a high degree of sequence identity shared between the receptor binding domains of the two toxin serotypes. The difference appears to be due in large part to a single lysine residue, K895, in BoNT/H, homologous to residue N905 in BoNT/A. The introduction of a bulky, positively charged residue at this position may cause a steric clash with ciA-C2. Exchanging the two residues between toxins (e.g. BoNT/A N905K and BoNT/H K895N) has been observed to lead to binding of BoNT/HA and a ˜30% loss of binding of BoNT/A. It was determined whether ciA-C2 could be evolved to restore binding to BoNT/A N905K, and, potentially, to bind variant BONT/H. Both the BoNT RBD and ciA-C2 VHH contain critical disulfides, making them good candidates for periplasmic selection.

Selection phage encoding ciA-C2 were evolved for 292 hours in four lagoons at increasing stringency towards binding wild-type BoNT/A RBD (residues 869-1296). Each lagoon discovered a divergent solution, yet all showed similar survival at high stringency (Fig. FIG. 21B). At least one combination of the point mutations discovered, variant Q12H F107L, performs roughly threefold better than ciA-C2, indicating potential for the selection to discover other beneficial mutations in ciA-C2, especially when paired with BoNT/A variant N905K RBD.

### Example 3

### Periplasmic PACE in Protease Evolution

The clan proline-alanine (PA) serine proteases are attractive candidates for reprogramming to generate therapeutically valuable new proteases. PA serine proteases are the best-studied of the serine protease clans, generally have highly efficient catalysis, and are involved in multiple biological processes vital to human health, including blood coagulation, apoptosis, and immunity. This example describes periplasmic PACE to evolve serine proteases with reprogrammed substrate specificity.

One embodiment of a periplasmic selection architecture for the reprogramming of disulfide-rich serine proteases is shown in FIG. 22. A binding domain comprised of two SH2 domains binds two HA4-CadC fusion moieties to create a CadC dimer. Cleavage of a desired substrate leads to removal of a degron tag from the binding domain; in the periplasm, the degron YjfN is used to induce proteolysis by the native periplasmic protease DegP. Additionally, a negative selection may be incorporated by placing an undesired substrate sequence between the two halves of the linker. Proteolytic cleavage of this sequence liberates singe SH2 domains, which can then compete with linked SH2 domains for HA4 binding.

### Equivalents and Scope

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

SEQUENCES

Signal sequence (SS)
AAACAAAGCACTATTGCACTGGCACTCTTACCGTTACTGTTTACCCCTGT
GACAAAAGCC (SEQ ID NO: 8)
SS_{PelB}
AAATACCTGCTGCCGACCGCTGCTGCTGGTCTGCTGCTCCTCGCTGCCCAACCGGCA
ATGGCC (SEQ ID NO: 9)
−600- +1 P_{CadBA}¹⁰⁵
TGCCGGAATTGAACAACCTGTCCATTATATATCAAATAAAAGCGGTCAGTGCTCTG
GTAAAAGGTAAAACAGATGAGTCTTACCAGGCGATAAATACTGGCATTGATCTTGA
AATGTCCTGGCTAAATTATGTGTTGCTTGGCAAGGTTTATGAAATGAAGGGGATGA
ACCGGGAAGCAGCTGATGCATATCTCACCGCCTTTAATTTACGCCCAGGGGCAAAC
ACCCTTTACTGGATTGAAAATGGTATATTCCAGACTTCTGTTCCTTATGTTGTACCTT
ATCTCGACAAATTTCTTGCTTCAGAATAAGTAACTCCGGGTTGATTTATGCTCGGAA
ATATTTGTTGTTGAGTTTTTGTATGTTCCTGTTGGTATAATATGTTGCGGCAATTTAT
TTGCCGCATAATTTTTATTACATAAATTTAACCAGAGAATGTCACGCAATCCATTGT
AAACATTAAATGTTTATCTTTTCATGATATCAACTTGCGATCCTGATGTGTTAATAA
AAAACCTCAAGTTCTCACTTACAGAAACTTTTGTGTTATTTCACCTAATCTTTAGGAT
TAATCCTTTTTTCGTGAGTAATCTTATCGCCA (SEQ ID NO: 10)
CadC₁₋₁₅₅¹⁰⁴
ATGCAACAACCTGTAGTTCGCGTTGGCGAATGGCTTGTTACTCCGTCCATAAACCAA
ATTAGCCGCAATGGGCGTCAACTTACCCTTGAGCCGAGATTAATCGATCTTCTGGTT
TTCTTTGCTCAACACAGTGGCGAAGTACTTAGCAGGGATGAACTTATCGATAATGTC
TGGAAGAGAAGTATTGTCACCAATCACGTTGTGACGCAGAGTATCTCAGAACTACG
TAAGTCATTAAAAGATAATGATGAAGATAGTCCTGTCTATATCGCTACTGTACCAAA
GCGCGGCTATAAATTAATGGTGCCGGTTATCTGGTACAGCGAAGAAGAGGGAGAGG
AAATAATGCTATCTTCGCCTCCCCCTATACCAGAGGCGGTTCCTGCCACAGATTCTC
CCTCCCACAGTCTTAACATTCAAAACACCGCAACGCCACCTGAACAATCCCCAGTTA
AAAGCAAACGA (SEQ ID NO: 11)
Leu(16)TM¹⁰⁴
GGCGGCCCAGGGTTACTGCTGTTACTGCTACTTCTTTTATTGTTATTACTGTTGTTAT
TGGGTCCAGGTGGC (SEQ ID NO: 12)
HA4
GGCAGCTCTGTGAGTAGCGTTCCGACCAAACTGGAAGTGGTTGCAGCAACCCCGAC
GAGCCTGCTGATTTCTTGGGATGCCCCGATGTCTAGTAGCTCTGTGTATTACTATCG
TATCACCTACGGTGAAACGGGCGGTAACAGCCCGGTGCAGGAATTTACGGTTCCGT
ATAGTAGCTCTACCGCGACGATTAGTGGCCTGAGCCCGGGTGTGGATTACACCATC
ACGGTTTATGCATGGGGCGAAGATAGCGCGGGTTACATGTTCATGTATTCTCCGATT
AGTATCAATTATCGTACCTGC (SEQ ID NO: 13)
SH2
AGTCTGGAAAAACACAGCTGGTATCATGGCCCTGTGAGCCGTAACGCGGCCGAATA
CCTGCTGAGCTCTGGCATTAATGGTTCTTTTCTGGTTCGTGAAAGTGAAAGTAGCCC
GGGCCAGCGCAGCATTTCTCTGCGTTATGAAGGTCGCGTGTATCACTACCGTATCAA
CACCGCCAGCGATGGCAAACTGTACGTTTCTAGTGAATCTCGCTTCAATACCCTGGC
AGAACTGGTGCATCACCATAGCACGGTTGCGGATGGTCTGATCACCACGCTGCATT
ATCCGGCGCCGAAACGC (SEQ ID NO: 14)
YibK. Position 139 is shown in bold.
ATGCTGGACATTGTCTTGTACGAACCTGAAATTCCGCAGAACACGGGCAACATCAT
TCGTTTGTGTGCAAACACAGGATTTCGTCTTCACTTAATCGAGCCGCTGGGGTTCAC
TTGGGATGACAAACGCCTTCGCCGTTCCGGGTTGGATTACCACGAGTTCGCCGAAAT
TAAACGCCACAAAACCTTTGAGGCTTTTCTGGAGAGCGAGAAACCTAAACGTTTGT
TTGCCCTTACCACCAAGGGATGCCCCGCTCATTCGCAAGTAAAGTTTAAATTAGGGG
ATTACCTGATGTTCGGCCCAGAGACACGCGGAATTCCCATGTCGATTCTTAATGAAA
TGCCGATGGAACAGAAGATCCGCATTCCGATGACCGCGAACTCGCGTTCCATGAAC
CTTAGCAATTCTGTCGCCGTGACAGTCTATGAGGCTTGGCGTCAATTAGGATATAA
GGGGGCAGTTAATCTGCCCGAGGTGAAA (SEQ ID NO: 15)
YibK variant 3.7. R139 is shown in bold. PANCE mutations are shown in underline.
ATGCTGGACATTGTCTTGTACGAACCTGAAATTCCGCAGAACACGGGCAACATCAT
TCGTTTGTGTGCAAACACAGGATTTCGTCTTCACTTAATCGAGCCGCTGGGGTTCAC
TTGGGATGACAAACGCCTTCGCCGTTCCGGGTTGGATTACCACGAGTTCGCCGAAAT
TAAACGCCACAAAACCTTTGAGGCTTTTCTGGAGAGCGAGAAACCTAAACGTTTGT
TTGCCCTTACCACCAAGGGATGCCCCGCTCATTCGCAAGTAAAGTTTAAATTAGGGG
ATTATCTGATGTTCGGCCCAGAGACACGCGGAATTCCCATGTCGATTCTTAATGAAA
TGCCGATGGAACAGAAGATCCGCATTCCGATGACCGCGAACTCGCGTTCCATGAAC
CTTAGCAATTCTGTCGATAGGACAGTCTATGAGGATTGGTGTCAATTAGGATATAA
GGGGGCAGTTAATCTGCCCGAGGTGAAA (SEQ ID NO: 16)
GCN4
TTGCAAAGAATGAAACAACTTGAAGACAAGGTTGAAGAATTGCTTTCGAAAAATTA
TCACTTGGAAAATGAGGTTGCCAGATTAAAGAAATTAGTTGGCGAACGC (SEQ ID
NO: 17)
GCN4(7P14P)
TTGCAAAGAATGAAACAACTTGAACCGAAGGTTGAAGAATTGCTTCCGAAAAATTA
TCACTTGGAAAATGAGGTTGCCAGATTAAAGAAATTAGTTGGCGAACGC (SEQ ID
NO: 18)
NpuN-SS₁₋₈
AAACAAAGCACTATTGCACTGTGTCTCAGCTACGAAACCGAAATCTTGACCGTCGA
ATATGGTCTGCTGCCAATCGGCAAGATTGTTGAAAAACGTATTGAATGTACGGTCTA
CTCAGTGGATAACAACGGCAATATCTACACCCAGCCGGTGGCCCAGTGGCATGACC
GTGGTGAACAGGAAGTGTTCGAATATTGTCTGGAAGACGGATCTTTAATCCGTGCC
ACAAAGGATCACAAATTTATGACTGTAGATGGTCAGATGCTCCCAATCGACGAAAT
TTTTGAACGCGAATTAGACCTGATGCGCGTGGATAATCTCCCGAAT (SEQ ID NO: 19)
NpuC-SS₉₋₂₀. SS₉₋₂₀ is shown in bold.
ATGATCAAAATTGCCACGCGTAAATATTTAGGCAAACAGAATGTTTATGATATCGG
TGTCGAGCGCGATCATAATTTCGCGCTGAAAAACGGCTTTATCGCCAGCAATTGTTT
TAATGCACTCTTACCGTTACTGTTTACCCCTGTGACTAAAGCC (SEQ ID NO: 20)
Ω-graft scFv. Positions 231 and 232 are shown in bold. Position 100 is shown
in underline.
CGCGACATTGTTATGACGCAGTCGCCATCAAGCTTATCAGCGTCAGTGGGAGATCG
CGTTACAATTACATGCCGTTCGAGCACTGGGGCAGTCACAACCAGTAATTACGCTTC
GTGGGTCCAGGAAAAACCGGGTAAGTTGTTCAAGGGTTTGATTGGCGGTACTAATA
ACCGCGCACCGGGCGTCCCTAGCCGTTTTTCGGGGAGTTTGATTGGTGACAAGGCC
ACACTTACTATCAGCAGTCTGCAACCAGAGGATTTCGCTACATACTTTTGTGCATTG
TGGTACTCCAACCATTGGGTCTTCGGTCAGGGCACGAAGGTTGAACTTAAACGCGG
GGGTGGTGGCTCCGGAGGTGGGGGTTCAGGCGGCGGAGGGTCTTCGGGTGGAGGG
AGTGAGGTTAAGCTTCTTGAAAGTGGTGGTGGTCTTGTGCAGCCTGGAGGCTCGTTA
AAGCTGAGCTGCGCTGTGAGTGGTTTCTCGTTGACGGATTATGGGGTCAATTGGGTA
CGCCAGGCACCGGGGCGTGGCTTGGAGTGGATTGGCGTCATCTGGGGCGACGGAAT
CACTGATTATAACAGTGCCTTGAAGGATCGCTTTATCATCAGCAAAGACGATTGCG
AAAACACTGTCTATTTGCAAATGAGCAAAGTTCGCTCGGATGATACGGCGTTATACT
ACTGTGTCACCGGACTTTTTGACTACTGGGGGCAGGGCACTCTTGTCACGGTCTCC
AGC (SEQ ID NO: 21)
Ω-graft scFv variant 3705c2.1. PACE mutations are shown in bold.
TGCGACATTGTTATGACGCAGTCGCCATCAAGCTTATCAGCGTCAGTGGGAGATCG
CGTTACAATTACATGCCGTTCGAGCACTGGGGCAGTCACAACCAGTAATTACGCTTC
GTGGGTCCAGGAAAAACCGGGTAAGTTGTTCAAGGGTTTGATTGGCGGTACTAATA
ACCGCGCACCGGGCGTCCCTAGCCGTTTTTCGGGGAGTTTGATTGGTGACAAGGCC
ACACTTACTATCAGCAGTCTGCAACCAGAGGATTTCGCTACATACTTTTGTGCATTG
TGGTACTCCAACCATTGGGTCTTCGGTCAGGGCACGAAGGTTGAACTTAAACGCGG
GGGTGGTGGCTCCGGAGGTGGGGGTTCAGGCGGCGGAGGGTCTTCGGGTGGAGGG
AGTGAGGTTAAGCTTCTTGAAAGTGGTGGTGGTCTTGTGCAGCCTGGAGGCTCGTTA
AAGCTGAGCTGCGCTGTGAGTGGTTTCTCGTTGACGGATTATGGGGTCAATTGGGTA
CGCCAGGCACCGGGGCGTGGCTTGGAGTGGATTGGCGTCATCTGGGGCGACGGAAT
CACTGATTATAATAGTGCCTTGAAGGATCGCTTTATCATCAGCAAAGACGATTGCG
AAAACACTGTCTATTTGCAAATGAGCAAAGTTCGCTCGGATGATACGGCGTTATAC
TACTGTGTCACCGGACTCGCTGACTACTGGGGGCAGGGCACTCTTGTCACGGTCTCC
AGC (SEQ ID NO: 22)
Ω-graft scFv variant 4004c4.2. PACE mutations are shown in bold.
TGCGACATTGTTATGACGCAGTCGCCATCAAGCTTATCAGCGTCAGTGGGAGATCG
CGTTACAATTACATGCCGTTCGAGCACTGGGGCAGTCACAACCAGTAATTACGCTTC
GTGGGTCCAGGAAAAACCGGGTAAGTTGTTCAAGGGTTTGATTGGCGGTACTAATA
ACCGCGCACCGGGCGTCCCTAGCCGTTTTTCGGGGAGTTTGATTGGTGACAAGGCC
ACACTTACTATCAGCAGTCTGCAACCAGAGGATTTCGCTACATACTTTTGTGCATTG
TGGTACTCCAACCATTGGGTCTTCGGTCAGGGCACGAAGGTTGAACTTAAACGCGG
GGGTGGTGGCTCCGGAGGTGGGGGTTCAGGCGGCGGAGGGTCTTCGGGTGGAGGG
AGTGAGGTTAAGCTTCTTGAAAGTGGTGGTGGTCTTGTGCAGCCTGGAGGCTCGTTA
AAGCTGAGCTGCGCTGTGAGTGGTTTCTCGTTGACGGATTATGGGGTCAATTGGGTA
CGCCAGGCACCGGGGCGTGGCTTGGAGTGGATTGGCGTCATCTGGGGCGACGGAAT
CACTGATTATAACAGTGCCTTGAAGGATCGCTTTATCATCAGCAAAGACGATTGCG
AAAACACTGTCTATTTGCAAATGAGCAAAGTTCGCTCGGATGATACGGCGTTATACT
ACTGTGTCACCGGATTAGCTGACTACTGGGGGCAGGGCACTCTTGTCACGGTCTCCA
GC (SEQ ID NO: 23)
Ω-graft scFv variant 4004c4.6. PACE mutations are shown in bold.
TGCGACAATGTTATGACGCAGTCGCCATCAAGCTTATCAGCGTCAGTGGGAGATCG
CGTTACAATTACATGCCGTTCGAGCACTGGGGCAGTCACAACCAGTAATTACGCTTC
GTGGGTCCAGGAAAAACCGGGTAAGGTGTTCAAGGGTTTGATTGGCGGTACTAATA
ACCGCGCACCGGGCGTCCCTAGCCGTTTTTCGGGGAGTTTGATTGGTGACAAGGCC
ACACTTACTATCAGCAGTCTGCAACCAGAGGATTTCGCTACATACTTTTGTGCATTG
TGGTACTCCAACCATTGGGTCTTCGGTCAGGGCACGAAGGTTGAACTTAAACGCGG
GGGTGGTGGCTCCGGAGGTGGGGGTTCAGGCGGCGGAGGGTCTTCGGGTGGAGGG
AGTGAGGTTAAGCTTCTTGAAAGTGGTGGTGGTCTTGTGCAGCCTGGAGGCTCGTTA
AAGCTGAGCTGCGCTGTGAGTGGTTTCTCGTTGACGGATTATGGGGTCAATTGGGTA
CGCCAGGCACCGGGGCGTGGCTTGGAGTGGATTGGCGTCATCTGGGGCGACGGAAT
CACTGATTATAACAGTGCCTTGAAGGATCGCTTTATCATCAGCAAAGACGATTGCG
AAAACACTGTCTATTTGCAAATGAGCAAAGTTCGCTCGGATGATACGGCGTCATAC
TACTGTGTCACCGGATTCGCTGACTACTGGGGGCAGGGCACTCTTGTCACGGTCTCC
AGC (SEQ ID NO: 24)
Ω-graft scFv variant 4004c4.8. PACE mutations are shown in bold.
TGCGACATTGTTATGACGCAGTCGCCATCAAGCTTATCAGCGTCAGTGGGAGATCG
CGTTACAATTACATGCCGTTCGAGCACTGGGGCAGTCACAACCAGTAATTACGCTTC
GTGGGTCCAGGAAAAACCGGGTAAGTTGTTCAAGGGTTTGATTGGCGGTACTAATA
ACCGCGCACCGGGCGTCCCTAGCCGTTTTTCGGGGAGTTTGATTGGTGACAAGGCC
ACACTTACTATCAGCAGTCTGCAACCAGAGGATTTCGCTACATACTTTTGTGCATTG
TGGTACTCCAACCATTGGGTCTTCGGTCAGGGCACGAAGGTTGAACTTAAACGCGG
GGGTGGTGGCTCCGGAGGTGGGGGTTCAGGCGGCGGAGGGTCTTCGGGTGGAGGG
AGTGAGGTTAAGCTTCTTGAAAGTGGTGGTGGTCTTGTGCAGCCTGGAGGCTCGTTA
AAGCTGAGCTGCGCTGTGAGTGGTTTCTCGTTGACGGATTATGGGGTCAATTGGGTA
CGCCAGGCACCGGGGCGTGGCTTGGAGTGGATTGGCGTCATCTGGGGCGACGGAAT
CACTGATTATAACAGTGCCTTGAAGGATCGCTTTATCATCAGCAAAGACGATTGCG
AAAACACTGTCTATTTGCAAATGAGCAAAGTTCGCTCGGATGATACGGCGTCATAC
TACTGTGTCACCGGATTAGCTGACTACTGGGGGCAGGGCACTCTTGTCACGGTCTCC
AGC (SEQ ID NO: 25)
Trastuzumab scFv.
GACATTCAGATGACGCAGTCGCCATCAAGCTTAAGCGCCAGTGTGGGTGATCGCGT
CACAATCACATGCCGTGCTTCCCAAGATGTAAATACCGCGGTGGCCTGGTATCAGC
AAAAACCGGGAAAAGCTCCGAAGCTTTTAATTTACAGTGCATCGTTCCTTTATAGCG
GGGTCCCAAGCCGCTTTTCGGGTTCGCGCTCCGGGACCGACTTCACGCTTACGATTT
CAAGCCTGCAACCGGAAGATTTCGCCACATACTATTGCCAACAGCATTACACGACG
CCGCCTACCTTCGGGCAAGGCACGAAGGTGGAAATCAAACGCGGGGGAGGTGGCT
CCGGAGGTGGGGGTTCAGGCGGCGGAGGGTCTTCGGGTGGAGGGAGTGAGGTTCA
GCTTGTGGAATCAGGTGGAGGTTTAGTGCAACCTGGTGGTAGTTTACGCCTGTCCTG
CGCAGCTAGTGGATTCAATATCAAAGACACTTATATCCATTGGGTACGTCAAGCCCC
TGGGAAAGGACTGGAATGGGTCGCCCGTATTTACCCCACTAACGGTTATACTCGTTA
CGCCGACTCTGTTAAGGGACGCTTCACCATTAGTGCGGACACATCTAAAAACACAG
CTTACTTGCAGATGAACTCCCTTCGTGCAGAGGACACCGCCGTCTACTACTGTAGCC
GTTGGGGAGGGGATGGATTTTATGCGATGGACTACTGGGGGCAGGGCACTCTTGTC
ACGGTCTCCAGC (SEQ ID NO: 26)
SS-Trastuzumab scFv variant 1.1. SS is highlighted in underline. Mutated
residues are shown in bold.
AAACAAAGCACTATTGCACTGGCACTCTTACCGTTACTGTTTATCCCTGTGACTAAA
GCCATGCGGGACATTCAGATGACGCAGTCGCCATCAAGCTTAAGCGCCAGTGTGGG
TGATCGCGTCACAATCACATGCCGTGCTTCCCAAGATGTAAATACCGCGGTGGCCTG
GTATCAGCAAAAACCGGGAAAAGCTCCGAAGCTTTTAATTTACAGTGCATCGTTCCT
TTATAGCGGGGTCCCAAGCCGTTTTTCGGGTTCGCGCTCCGGGACCGACTTCACGCT
TACGATTTCAAGCCTGCAACCGGAAGATTTCGCCACATACTATTGCCAACAGTATTA
CACGACGCCGCCTACCTTCGGGCAAGGCACGAAGGTGGAAATCAAACGCGGGGGA
GGTGGCTCCGGAGGTGGGGGTTCAGGCGGCGGAGGGTCTTCGGGTGGAGGGAGTG
AGGTTCAGCTTGTGGAATCAGGTGGAGGTTTAGTGCAACCTGGTGGTAGTTTACGCC
TGTCCTGCGCAGCTAGTGGATTCAATATCAAAGACACTTATATCCATTGGGTACGTC
AAGCCCCTGGGAAAGGACTGGAATGGGTCGCCCGTATTTACCCCACTAACGGTTAT
ACTCGTTACGCCGACTCTGTTAAGGGACGCTTCACCATTAGTGCGGACACATCTAAA
AACACAGCTTACTTGCAGATGAACTCCCTTCGTGCAGAGGACACCGCCGTCTACTAC
TGTAGCCGTTGGGGAGGGGATGGATTTTATGCGATGGACTACTGGGGGCAGGGCAC
TCTTGTCACGGTCTCCAGC (SEQ ID NO: 27)
NpuC-SS9-20-Trastuzumab scFv variant 3.2. SS9-20 is highlighted in underline.
Mutated residues are shown in bold.
ATGATCAAAATTGCCACGCGTAAATATTTAGGCAAACAGAATGTTTATGATATCGG
TGTCGAGCGCGATCATAATTTCGCGCTGAAAAACGGCTTTATCGCCAGCAATTGTTT
TAATGCACTCTTACCGTTACTGTTTACCCCTGTGACTAAAGACATGCGGGACATTCA
GATGACGCAGTCGCCATCAAGCTTAAGCGCCAGTGTGGGTGATCGCGTCACAATCA
CATGCCGTGCTTCCCAAGATGTAAATACCGCGGTGGACTGGTATCAGCAAAAACCG
GGAAAAGCTCCGAAGCTTTTAATTTCCAGTGCATCGTTCCTTTATAGCGGGGTCCCA
AGCCGCTTTTCGGGTTCGCGCTCCGGGACCGACTTCACGCTTACGATTTCAAGCCTG
CAACCGGAAGATTTCGCCACATACTATTGCCAACAGCATTACACGACGCCGCCTAC
CTTCGGGCAAGGCACGAAGGTGGAAATCAAACGCGGGGGAGGTGGCTCCGGAGGT
GGGGGTTCAGGCGGCGGAGGGTCTTCGGGTGGAGGGAGTGAGGTTCAGCTTGTGGA
ATCAGGTGGAGGTTTAGTGCAACCTGGTGGTAGTTTACGCCTGTCCTGCGCAGCTAG
TGGATTCAATATCAAAGACACTTATATCCATTGGGTACGTCAAGCCCCTGGGAAAG
GACTGGAATGGGTCGCCCGTATTTACCCCACTAACGGTTATACTCGTTACGCCGACT
CTGTTAAGGGACGCTTCACCATTAGTGCGGACACATCTAAAAACACAGCTTACTTGC
AGATGAACTCCCTTCGTGCAGAGGACACCGCCGTCTACTACTGTAGCCGTTGGGGA
GGGGATGGATTTTATGCGATGGACTACTGGGGGCAGGGCACTCTTGTCACGGTCTC
CAGCGGTGGATCAGGCGGAAGTGGCGGTTCAGGTGGGAGTGGTGGCAGCTTGCAA
AGAATGAAACAACTTGAAGACAAGGTTGAAGAATTGCTTTCGAAAAATTATCACTT
GGAAAATGAGGTTGCCAGATTAAAGAAATTAGTTGGCGAACGC (SEQ ID NO: 28)
Trastuzumab scFv with disulfide-forming Cys residues converted to Ser. Modified
codons are shown in bold.
GACATTCAGATGACGCAGTCGCCATCAAGCTTAAGCGCCAGTGTGGGTGATCGCGT
CACAATCACATCCCGTGCTTCCCAAGATGTAAATACCGCGGTGGCCTGGTATCAGC
AAAAACCGGGAAAAGCTCCGAAGCTTTTAATTTACAGTGCATCGTTCCTTTATAGCG
GGGTCCCAAGCCGCTTTTCGGGTTCGCGCTCCGGGACCGACTTCACGCTTACGATTT
CAAGCCTGCAACCGGAAGATTTCGCCACATACTATTCCCAACAGCATTACACGACG
CCGCCTACCTTCGGGCAAGGCACGAAGGTGGAAATCAAACGCGGGGGAGGTGGCT
CCGGAGGTGGGGGTTCAGGCGGCGGAGGGTCTTCGGGTGGAGGGAGTGAGGTTCA
GCTTGTGGAATCAGGTGGAGGTTTAGTGCAACCTGGTGGTAGTTTACGCCTGTCCTC
AGCAGCTAGTGGATTCAATATCAAAGACACTTATATCCATTGGGTACGTCAAGCCC
CTGGGAAAGGACTGGAATGGGTCGCCCGTATTTACCCCACTAACGGTTATACTCGTT
ACGCCGACTCTGTTAAGGGACGCTTCACCATTAGTGCGGACACATCTAAAAACACA
GCTTACTTGCAGATGAACTCCCTTCGTGCAGAGGACACCGCCGTCTACTACTCTAGC
CGTTGGGGAGGGGATGGATTTTATGCGATGGACTACTGGGGGCAGGGCACTCTTGT
CACGGTCTCCAGC (SEQ ID NO: 29)
H98 mimetic peptide.
CTTCTGGGGCCATACGAATTATGGGAATTAAGTCAC (SEQ ID NO: 30)
5X GGS linker. Used to link YibK or scFv to C-terminal GCN4, SH2 or YibK.
GGTGGATCAGGCGGAAGTGGCGGTTCAGGTGGGAGTGGTGGCAGC (SEQ ID NO: 31)
CadC
MQQPVVRVGEWLVTPSINQISRNGRQLTLEPRLIDLLVFFAQHSGEVLSRDELIDNVWK
RSIVTNHVVTQSISELRKSLKDNDEDSPVYIATVPKRGYKLMVPVIWYSEEEGEEIMLSS
PPPIPEAVPATDSPSHSLNIQNTATPPEQSPVKSKRFTTFWVWFFFLLSLGICVALVAFSSL
DTRLPMSKSRILLNPRDIDINMVNKSCNSWSSPYQLSYAIGVGDLVATSLNTFSTFMVH
DKINYNIDEPSSSGKTLSIAFVNQRQYRAQQCFMSIKLVDNADGSTMLDKRYVITNGNQ
LAIQNDLLESLSKALNQPWPQRMQETLQKILPHRGALLTNFYQAHDYLLHGDDKSLNR
ASELLGEIVQSSPEFTYARAEKALVDIVRHSQHPLDEKQLAALNTEIDNIVTLPELNNLSII
YQIKAVSALVKGKTDESYQAINTGIDLEMSWLNYVLLGKVYEMKGMNREAADAYLTA
FNLRPGANTLYWIENGIFQTSVPYVVPYLDKFLASE (SEQ ID NO: 33)

What is claimed is:

1. A method of continuous evolution comprising:
(a) contacting a population of bacterial host cells in a culture medium with a population of selection phage comprising a gene of interest to be evolved and lacking a functional pIII gene required for the generation of infectious phage particles; wherein
(1) the phage allow for expression of the gene of interest in the host cells;
(2) the host cells are suitable host cells for phage infection, replication, and packaging, wherein the phage comprises all phage genes required for the generation of phage particles, except a full-length pIII gene; and
(3) the host cells comprise:
(i) a first expression construct encoding a fusion protein comprising a DNA binding protein connected to a periplasmic capture agent; and
(ii) a second expression construct encoding a pIII protein under the control of a conditional promoter, wherein activation of the conditional promoter is dependent on binding of a first gene product of the gene of interest to the periplasmic capture agent; and
(b) incubating the population of host cells under conditions allowing for the mutation of the gene of interest, the production of infectious phage, and the infection of host cells with phage, wherein infected cells are removed from the population of host cells, and wherein the population of host cells is replenished with fresh host cells that are not infected by phage, wherein the binding of the first gene product to the periplasmic capture agent is a desired function, wherein phage expressing gene products having a desired function induce production of pIII and release progeny into the culture medium capable of infecting new host cells, and wherein phage expressing gene products having an undesired function do not produce pIII and release only non-infectious progeny into the culture medium.

2. The method of claim 1, wherein the population of bacterial host cells comprises a population of E. coli cells.

3. The method of claim 1 or claim 2, wherein the selection phage are filamentous phage.

4. The method of any one of claims 1 to 3, wherein the selection phage are M13 phage.

5. The method of any one of claims 1 to 4, wherein the gene of interest to be evolved encodes a protein.

6. The method of any one of claims 1 to 5, wherein the protein comprises one or more disulfide bonds.

7. The method of claim 5 or 6, wherein the protein is an antibody, antibody fragment, or single-chain variable region (scFv), single-domain antibody, extracellular receptor, extracellular protease, monobody, adnectin, or nanobody.

8. The method of any one of claims 5 to 7, wherein the protein further comprises a capture tag.

9. The method of claim 8, wherein the capture tag comprises a peptide.

10. The method of claim 8 or 9, wherein the capture tag comprises a SH2 domain or a GCN4 leucine zipper domain.

11. The method of any one of claims 1 to 11, wherein the DNA binding protein is a bacterial DNA binding protein.

12. The method of claim 11, wherein the bacterial DNA binding protein comprises a CadC protein (SEQ ID NO: 33) or a fragment thereof.

13. The method of claim 11 or 12, wherein the DNA binding protein lacks a periplasmic sensor domain.

14. The method of any one of claims 11 to 13, wherein the DNA binding protein is encoded by the nucleic acid sequence set forth in SEQ ID NO: 11.

15. The method of any one of claims 1 to 14, wherein the periplasmic capture agent comprises a cognate binding partner of the first gene product.

16. The method of any one of claims 1 to 15, wherein the periplasmic capture agent comprises an antigen that binds the first gene product.

17. The method of any one of claims 1 to 16, wherein the periplasmic capture agent comprises an antibody or fragment thereof that binds to the first gene product.

18. The method of any one of claims 1 to 17, wherein the periplasmic capture agent comprises a monobody that binds to the first gene product.

19. The method of any one of claims 1 to 19, wherein the first expression construct further comprises a nucleic acid sequence encoding a portion of a split-intein.

20. The method of claim 19, wherein the portion of the split-intein is connected to a portion of a periplasmic signal peptide sequence.

21. The method of claim 20, wherein the portion of the periplasmic signal peptide sequence encodes amino acids 1-8 of SEQ ID NO: 32.

22. The method of any one of claims 19 to 21, wherein the split-intein comprises a Nostoc punctiforme (Npu) trans-splicing DnaE intein N-terminal portion or C-terminal portion.

23. The method of any one of claims 19 to 22, wherein the split-intein is encoded by the nucleic acid sequence set forth in SEQ ID NO: 19.

24. The method of any one of claims 19 to 23, wherein the selection phage further comprises a nucleic acid sequence encoding a portion of a split-intein connected to the gene of interest to be evolved.

25. The method of claim 24, wherein the portion of the split-intein is connected to a portion of a periplasmic signal peptide sequence.

26. The method of claim 25, wherein the portion of the periplasmic signal peptide sequence encodes amino acids 9-20 of SEQ ID NO: 32.

27. The method of any one of claims 19 to 26, wherein the split-intein comprises a Nostoc punctiforme (Npu) trans-splicing DnaE intein N-terminal portion or C-terminal portion.

28. The method of any one of claims 19 to 27, wherein the split-intein is encoded by the nucleic acid sequence set forth in SEQ ID NO: 20.

29. The method of any one of claims 1 to 28, wherein the conditional promoter comprises two or more DNA binding protein binding sites.

30. The method of claim 29, wherein the two or more binding sites comprise a Cad1 binding site and a Cad2 binding site.

31. The method of claim 29 or 30, wherein the conditional promoter comprises a P_{cadBA} promoter.

32. The method of any one of claims 29 to 31, wherein the conditional promoter comprises the sequence set forth in SEQ ID NO: 10.

33. The method of any one of claims 1 to 32, wherein the host cells further comprise a mutagenesis plasmid.

34. The method of any one of claims 1 to 33, wherein the first expression construct and the second expression construct are situated on the same vector.

35. The method of claim 34, wherein the vector is a bacterial plasmid.

36. The method of any one of claims 1 to 35 wherein the first expression construct and the second expression construct are situated on different vectors.

37. The method of claim 36, wherein each vector is a bacterial plasmid.

38. The method of any one of claims 1 to 37, further comprising isolating the first gene product from the population of host cells.

39. A protein evolved by the method of any one of claims 1 to 38.

40. An isolated nucleic acid comprising sequence, or encoding a protein having the sequence, as set forth in any one of SEQ ID NO: 1-33.

41. An apparatus for continuous evolution of a gene of interest, the apparatus comprising
(a) a lagoon comprising
a cell culture vessel comprising population of bacterial host cells in a culture medium with a population of selection phage comprising a gene of interest to be evolved and lacking a functional pIII gene required for the generation of infectious phage particles;
wherein
(1) the phage allow for expression of the gene of interest in the host cells;
(2) the host cells are suitable host cells for phage infection, replication, and packaging, wherein the phage comprises all phage genes required for the generation of phage particles, except a full-length pIII gene; and
(3) the host cells comprise:
(i) a first expression construct encoding a fusion protein comprising a DNA binding protein connected to a periplasmic capture agent; and
(ii) a second expression construct encoding a pIII protein under the control of a conditional promoter, wherein activation of the conditional promoter is dependent on binding of a first gene product of the gene of interest to the periplasmic capture agent;
an inflow connected to a turbidostat;
optionally an inflow, connected to a vessel comprising a mutagen; optionally an inflow, connected to a vessel comprising an inducer;
an outflow;
a controller controlling inflow and outflow rates
(b) a turbidostat comprising
a cell culture vessel comprising a population of fresh bacterial host cells;
an outflow connected to the inflow of the lagoon;
an inflow connected to a vessel comprising liquid media
a turbidity meter measuring the turbidity of the culture of fresh bacterial host cells in the turbidostat;
a controller controlling the inflow of sterile liquid media and the outflow into the waste vessel based on the turbidity of the culture liquid;
(c) optionally, a vessel comprising mutagen; and
(d) optionally, a vessel comprising an inducer.

42. The apparatus of claim 41, wherein the phages are M13 phages.

43. The apparatus of claim 42, wherein the M13 phages do not comprise a full-length pIII gene.

44. The apparatus of any one of claims 41 to 43, wherein the bacterial host cells are amenable to phage infection, replication, and production.

45. The apparatus of any one of claims 41 to 44, wherein the host cells are E. coli cells.

46. The apparatus of any one of claims 41 to 45, wherein the fresh host cells are not infected by the phage.

47. The apparatus of any one of claims 41 to 46, wherein the population of host cells is in suspension culture in liquid media.

48. The apparatus of any one of claims 41 to 47, wherein the rate of inflow of fresh host cells and the rate of outflow are substantially the same.

49. The apparatus of any one of claims 41 to 48, wherein the rate of inflow and/or the rate of outflow is from about 0.1 lagoon volumes per hour to about 25 lagoon volumes per hour.

50. The apparatus of any one of claims 41 to 49, wherein the inflow and outflow rates are controlled based on a quantitative assessment of the population of host cells in the lagoon.

51. The apparatus of claim 50, wherein the quantitative assessment comprises measuring of cell number, cell density, wet biomass weight per volume, turbidity, or growth rate.

52. The apparatus of any one of claims 41 to 51, wherein the inflow and/or outflow rate is controlled to maintain a host cell density of from about 10² cells/ml to about 10¹² cells/ml in the lagoon.

53. The apparatus of claim 52, wherein the inflow and/or outflow rate is controlled to maintain a host cell density of about 10² cells/ml, about 10³ cells/ml, about 10⁴ cells/ml, about 10⁵ cells/ml, about 5·10⁵ cells/ml, about 10⁶ cells/ml, about 5·10⁶ cells/ml, about 10⁷ cells/ml, about 5·10⁷ cells/ml, about 10⁸ cells/ml, about 5·10⁸ cells/ml, about 10⁹ cells/ml, about 5·10⁹ cells/ml, about 10¹⁰ cells/ml, about 5·10¹⁰ cells/ml, or more than 10¹⁰ cells/ml, in the lagoon.

54. The apparatus of claim 41, wherein the inflow and outflow rates are controlled to maintain a substantially constant number of host cells in the lagoon.

55. The apparatus of claim 41, wherein the inflow and outflow rates are controlled to maintain a substantially constant frequency of fresh host cells in the lagoon.

56. The apparatus of claim 41, wherein the population of host cells is continuously replenished with fresh host cells that are not infected by the phage.

57. The apparatus of any one of claims 41 to 56, wherein the lagoon further comprises an inflow connected to a vessel comprising a mutagen, and wherein the inflow of mutagen is controlled to maintain a concentration of the mutagen in the lagoon that is sufficient to induce mutations in the host cells.

58. The apparatus of claim 57, wherein the mutagen is ionizing radiation, ultraviolet radiation, base analogs, deaminating agents (e.g., nitrous acid), intercalating agents (e.g., ethidium bromide), alkylating agents (e.g., ethylnitrosourea), transposons, bromine, azide salts, psoralen, benzene,3-Chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl)acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide (captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-Aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9) or t-butyl hydroperoxide (BHP) (CAS no. 75-91-2).

59. The apparatus of any one of claims 41 to 58, wherein the lagoon comprises an inflow connected to a vessel comprising an inducer.

60. The apparatus of claim 59, wherein the inducer induces expression of mutagenesis-promoting genes into host cells.

61. The apparatus of any one of claims 41 to 60, wherein the host cells comprise an expression cassette encoding a mutagenesis-promoting gene under the control of an inducible promoter.

62. The apparatus of claim 61, wherein the inducible promoter is an arabinose-inducible inducer and wherein the inducer is arabinose.

63. The apparatus of any one of claims 41 to 62, wherein the lagoon volume is from approximately 1 ml to approximately 1001.

64. The apparatus of any one of claims 41 to 63, wherein the lagoon further comprises a heater and a thermostat controlling the temperature in the lagoon.

65. The apparatus of claim 64, wherein the temperature in the lagoon is controlled to be about 37° C.

66. The apparatus of any one of claims 41 to 65, wherein the inflow rate and/or the outflow rate are controlled to allow for the incubation and replenishment of the population of host cells for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive phage life cycles.

67. The apparatus of claim 66, wherein the time sufficient for one phage life cycle is about 10 minutes.

68. A vector system for periplasmic phage-based continuous directed evolution comprising
(a) selection phage comprising a gene of interest to be evolved and lacking a functional pIII gene required for the generation of infectious phage particles;
(b) a first expression construct encoding a fusion protein comprising a DNA binding protein connected to a periplasmic capture agent;
(c) a second expression construct encoding a pIII protein under the control of a conditional promoter, wherein activation of the conditional promoter is dependent on binding of a first gene product of the gene of interest to the periplasmic capture agent.

69. The vector system of claim 68, wherein the selection phage is an M13 phage.

70. The vector system of claim 68, wherein the selection phage comprises all genes required for the generation of phage particles.

71. The vector system of claim 68, wherein the phage genome comprises a pI, pII, pIV, pV, pVI, pVII, pVIII, pIX, and a pX gene, but not a full-length pIII gene.

72. The vector system of claim 68, wherein the phage genome comprises an F1 origin of replication.

73. The vector system of claim 68, wherein the phage genome comprises a 3′-fragment of a pIII gene.

74. The vector system of claim 68, wherein the 3′-fragment of the pIII gene comprises a promoter.

75. The vector system of claim 68, wherein the selection phage comprises a multiple cloning site operably linked to a promoter.

76. The vector system of any one of claims 68 to 75, wherein the gene of interest to be evolved encodes a protein.

77. The vector system of any one of claims 68 to 76, wherein the protein comprises one or more disulfide bonds.

78. The vector system of claim 68 or 77, wherein the protein is an antibody, antibody fragment, or single-chain variable region (scFv), single-domain antibody, extracellular receptor, extracellular protease, monobody, adnectin, or nanobody.

79. The vector system of any one of claims 68 to 78, wherein the protein further comprises a capture tag.

80. The vector system of claim 79, wherein the capture tag comprises a peptide.

81. The vector system of claim 79 or 80, wherein the capture tag comprises a SH2 domain or a GCN4 leucine zipper domain.

82. The vector system of any one of claims 68 to 81, wherein the DNA binding protein is a bacterial DNA binding protein.

83. The vector system of claim 82, wherein the bacterial DNA binding protein comprises a CadC protein (SEQ ID NO: 33) or a fragment thereof.

84. The vector system of claim 82 or 83, wherein the DNA binding protein lacks a periplasmic sensor domain.

85. The vector system of any one of claims 82 to 84, wherein the DNA binding protein is encoded by the nucleic acid sequence set forth in SEQ ID NO: 11.

86. The vector system of any one of claims 68 to 85, wherein the periplasmic capture agent comprises a cognate binding partner of the first gene product.

87. The vector system of any one of claims 68 to 86, wherein the periplasmic capture agent comprises an antigen that binds the first gene product.

88. The vector system of any one of claims 68 to 87, wherein the periplasmic capture agent comprises an antibody or fragment thereof that binds to the first gene product.

89. The vector system of any one of claims 68 to 88, wherein the periplasmic capture agent comprises a monobody that binds to the first gene product.

90. The vector system of any one of claims 68 to 89, wherein the first expression construct further comprises a nucleic acid sequence encoding a portion of a split-intein.

91. The vector system of claim 90, wherein the portion of the split-intein is connected to a portion of a periplasmic signal peptide sequence.

92. The vector system of claim 90 or 91, wherein the portion of the periplasmic signal peptide sequence encodes amino acids 1-8 of SEQ ID NO: 32.

93. The vector system of any one of claims 90 to 92, wherein the split-intein comprises a Nostoc punctiforme (Npu) trans-splicing DnaE intein N-terminal portion or C-terminal portion.

94. The vector system of any one of claims 90 to 93, wherein the split-intein is encoded by the nucleic acid sequence set forth in SEQ ID NO: 19.

95. The vector system of any one of claims 90 to 94, wherein the selection phage further comprises a nucleic acid sequence encoding a portion of a split-intein connected to the gene of interest to be evolved.

96. The vector system of claim 95, wherein the portion of the split-intein is connected to a portion of a periplasmic signal peptide sequence.

97. The vector system of claim 96, wherein the portion of the periplasmic signal peptide sequence encodes amino acids 9-20 of SEQ ID NO: 32.

98. The vector system of any one of claims 95 to 97, wherein the split-intein comprises a Nostoc punctiforme (Npu) trans-splicing DnaE intein N-terminal portion or C-terminal portion.

99. The vector system of any one of claims 95 to 98, wherein the split-intein is encoded by the nucleic acid sequence set forth in SEQ ID NO: 20.

100. The vector system of any one of claims 68 to 99, wherein the conditional promoter comprises two or more DNA binding protein binding sites.

101. The vector system of claim 100, wherein the two or more binding sites comprise a Cad1 binding site and a Cad2 binding site.

102. The vector system of claim 100 or 101, wherein the conditional promoter comprises a P_{cadBA} promoter.

103. The vector system of any one of claims 100 to 102, wherein the conditional promoter comprises the sequence set forth in SEQ ID NO: 10.

104. The vector system of any one of claims 68 to 103, wherein the vector system further comprises a mutagenesis plasmid.

105. The vector system of claim 104, wherein the mutagenesis plasmid comprises a gene expression cassette encoding a mutagenesis-promoting gene product.

106. The vector system of claim 105, wherein the expression cassette comprises a conditional promoter, the activity of which depends on the presence of an inducer.

107. The vector system of claim 106, wherein the conditional promoter is an arabinose-inducible promoter and the inducer is arabinose.


SEQUENCE LISTING (43 sequences)

SEQ ID NO: 1 - DNA, 32 bp, synthetic construct
  source 1..32: mol_type = other DNA
taatggaaacttcctcatgaaaaagtctttag

SEQ ID NO: 2 - DNA, 30 bp, synthetic construct
  source 1..30: mol_type = other DNA
acagagagaataacataaaaacagggaagc

SEQ ID NO: 3 - DNA, 70 bp, synthetic construct
  source 1..70: mol_type = other DNA
tgtggcaattatcattgcatcattcccttttcgaatgagtttctattatgtgtaggctggagctgcttcg

SEQ ID NO: 4 - DNA, 70 bp, synthetic construct
  source 1..70: mol_type = other DNA
tggcaagccacttcccttgtacgagctaattattttttgctttcttctttattccggggatccgtcgacc

SEQ ID NO: 5 - DNA, 22 bp, synthetic construct
  source 1..22: mol_type = other DNA
aaaataacgtcttgcattcacc

SEQ ID NO: 6 - DNA, 22 bp, synthetic construct
  source 1..22: mol_type = other DNA
ttcatgtgttctccttatgagc

SEQ ID NO: 7 - protein, 17 aa, synthetic construct
GGGGSLLGPYELWELSH

SEQ ID NO: 8 - DNA, 60 bp, synthetic construct
  source 1..60: mol_type = other DNA
aaacaaagcactattgcactggcactcttaccgttactgtttacccctgtgacaaaagcc

SEQ ID NO: 9 - DNA, 63 bp, synthetic construct
  source 1..63: mol_type = other DNA
aaatacctgctgccgaccgctgctgctggtctgctgctcctcgctgcccaaccggcaatggcc

SEQ ID NO: 10 - DNA, 601 bp, synthetic construct
  source 1..601: mol_type = other DNA
tgccggaattgaacaacctgtccattatatatcaaataaaagcggtcagtgctctggtaaaaggtaaaacagatgagtcttaccaggcgataaatactggcattgatcttgaaatgtcctggctaaattatgtgttgcttggcaaggtttatgaaatgaaggggatgaaccgggaagcagctgatgcatatctcaccgcctttaatttacgcccaggggcaaacaccctttactggattgaaaatggtatattccagacttctgttccttatgttgtaccttatctcgacaaatttcttgcttcagaataagtaactccgggttgatttatgctcggaaatatttgttgttgagtttttgtatgttcctgttggtataatatgttgcggcaatttatttgccgcataatttttattacataaatttaaccagagaatgtcacgcaatccattgtaaacattaaatgtttatcttttcatgatatcaacttgcgatcctgatgtgttaataaaaaacctcaagttctcacttacagaaacttttgtgttatttcacctaatctttaggattaatccttttttcgtgagtaatcttatcgcca

SEQ ID NO: 11 - DNA, 465 bp, synthetic construct
  source 1..465: mol_type = other DNA
atgcaacaacctgtagttcgcgttggcgaatggcttgttactccgtccataaaccaaattagccgcaatgggcgtcaacttacccttgagccgagattaatcgatcttctggttttctttgctcaacacagtggcgaagtacttagcagggatgaacttatcgataatgtctggaagagaagtattgtcaccaatcacgttgtgacgcagagtatctcagaactacgtaagtcattaaaagataatgatgaagatagtcctgtctatatcgctactgtaccaaagcgcggctataaattaatggtgccggttatctggtacagcgaagaagagggagaggaaataatgctatcttcgcctccccctataccagaggcggttcctgccacagattctccctcccacagtcttaacattcaaaacaccgcaacgccacctgaacaatccccagttaaaagcaaacga

SEQ ID NO: 12 - DNA, 72 bp, synthetic construct
  source 1..72: mol_type = other DNA
ggcggcccagggttactgctgttactgctacttcttttattgttattactgttgttattgggtccaggtggc

SEQ ID NO: 13 - DNA, 303 bp, synthetic construct
  source 1..303: mol_type = other DNA
ggcagctctgtgagtagcgttccgaccaaactggaagtggttgcagcaaccccgacgagcctgctgatttcttgggatgccccgatgtctagtagctctgtgtattactatcgtatcacctacggtgaaacgggcggtaacagcccggtgcaggaatttacggttccgtatagtagctctaccgcgacgattagtggcctgagcccgggtgtggattacaccatcacggtttatgcatggggcgaagatagcgcgggttacatgttcatgtattctccgattagtatcaattatcgtacctgc

SEQ ID NO: 14 - DNA, 300 bp, synthetic construct
  source 1..300: mol_type = other DNA
agtctggaaaaacacagctggtatcatggccctgtgagccgtaacgcggccgaatacctgctgagctctggcattaatggttcttttctggttcgtgaaagtgaaagtagcccgggccagcgcagcatttctctgcgttatgaaggtcgcgtgtatcactaccgtatcaacaccgccagcgatggcaaactgtacgtttctagtgaatctcgcttcaataccctggcagaactggtgcatcaccatagcacggttgcggatggtctgatcaccacgctgcattatccggcgccgaaacgc

SEQ ID NO: 15 - DNA, 480 bp, synthetic construct
  source 1..480: mol_type = other DNA
atgctggacattgtcttgtacgaacctgaaattccgcagaacacgggcaacatcattcgtttgtgtgcaaacacaggatttcgtcttcacttaatcgagccgctggggttcacttgggatgacaaacgccttcgccgttccgggttggattaccacgagttcgccgaaattaaacgccacaaaacctttgaggcttttctggagagcgagaaacctaaacgtttgtttgcccttaccaccaagggatgccccgctcattcgcaagtaaagtttaaattaggggattacctgatgttcggcccagagacacgcggaattcccatgtcgattcttaatgaaatgccgatggaacagaagatccgcattccgatgaccgcgaactcgcgttccatgaaccttagcaattctgtcgccgtgacagtctatgaggcttggcgtcaattaggatataagggggcagttaatctgcccgaggtgaaa

SEQ ID NO: 16 - DNA, 480 bp, synthetic construct
  source 1..480: mol_type = other DNA
atgctggacattgtcttgtacgaacctgaaattccgcagaacacgggcaacatcattcgtttgtgtgcaaacacaggatttcgtcttcacttaatcgagccgctggggttcacttgggatgacaaacgccttcgccgttccgggttggattaccacgagttcgccgaaattaaacgccacaaaacctttgaggcttttctggagagcgagaaacctaaacgtttgtttgcccttaccaccaagggatgccccgctcattcgcaagtaaagtttaaattaggggattatctgatgttcggcccagagacacgcggaattcccatgtcgattcttaatgaaatgccgatggaacagaagatccgcattccgatgaccgcgaactcgcgttccatgaaccttagcaattctgtcgataggacagtctatgaggattggtgtcaattaggatataagggggcagttaatctgcccgaggtgaaa

SEQ ID NO: 17 - DNA, 105 bp, synthetic construct
  source 1..105: mol_type = other DNA
ttgcaaagaatgaaacaacttgaagacaaggttgaagaattgctttcgaaaaattatcacttggaaaatgaggttgccagattaaagaaattagttggcgaacgc

SEQ ID NO: 18 - DNA, 105 bp, synthetic construct
  source 1..105: mol_type = other DNA
ttgcaaagaatgaaacaacttgaaccgaaggttgaagaattgcttccgaaaaattatcacttggaaaatgaggttgccagattaaagaaattagttggcgaacgc

SEQ ID NO: 19 - DNA, 327 bp, synthetic construct
  source 1..327: mol_type = other DNA
aaacaaagcactattgcactgtgtctcagctacgaaaccgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaat

SEQ ID NO: 20 - DNA, 156 bp, synthetic construct
  source 1..156: mol_type = other DNA
atgatcaaaattgccacgcgtaaatatttaggcaaacagaatgtttatgatatcggtgtcgagcgcgatcataatttcgcgctgaaaaacggctttatcgccagcaattgttttaatgcactcttaccgttactgtttacccctgtgactaaagcc

SEQ ID NO: 21 - DNA, 735 bp, synthetic construct
  source 1..735: mol_type = other DNA
cgcgacattgttatgacgcagtcgccatcaagcttatcagcgtcagtgggagatcgcgttacaattacatgccgttcgagcactggggcagtcacaaccagtaattacgcttcgtgggtccaggaaaaaccgggtaagttgttcaagggtttgattggcggtactaataaccgcgcaccgggcgtccctagccgtttttcggggagtttgattggtgacaaggccacacttactatcagcagtctgcaaccagaggatttcgctacatacttttgtgcattgtggtactccaaccattgggtcttcggtcagggcacgaaggttgaacttaaacgcgggggtggtggctccggaggtgggggttcaggcggcggagggtcttcgggtggagggagtgaggttaagcttcttgaaagtggtggtggtcttgtgcagcctggaggctcgttaaagctgagctgcgctgtgagtggtttctcgttgacggattatggggtcaattgggtacgccaggcaccggggcgtggcttggagtggattggcgtcatctggggcgacggaatcactgattataacagtgccttgaaggatcgctttatcatcagcaaagacgattgcgaaaacactgtctatttgcaaatgagcaaagttcgctcggatgatacggcgttatactactgtgtcaccggactttttgactactgggggcagggcactcttgtcacggtctccagc

SEQ ID NO: 22 - DNA, 735 bp, synthetic construct
  source 1..735: mol_type = other DNA
tgcgacattgttatgacgcagtcgccatcaagcttatcagcgtcagtgggagatcgcgttacaattacatgccgttcgagcactggggcagtcacaaccagtaattacgcttcgtgggtccaggaaaaaccgggtaagttgttcaagggtttgattggcggtactaataaccgcgcaccgggcgtccctagccgtttttcggggagtttgattggtgacaaggccacacttactatcagcagtctgcaaccagaggatttcgctacatacttttgtgcattgtggtactccaaccattgggtcttcggtcagggcacgaaggttgaacttaaacgcgggggtggtggctccggaggtgggggttcaggcggcggagggtcttcgggtggagggagtgaggttaagcttcttgaaagtggtggtggtcttgtgcagcctggaggctcgttaaagctgagctgcgctgtgagtggtttctcgttgacggattatggggtcaattgggtacgccaggcaccggggcgtggcttggagtggattggcgtcatctggggcgacggaatcactgattataatagtgccttgaaggatcgctttatcatcagcaaagacgattgcgaaaacactgtctatttgcaaatgagcaaagttcgctcggatgatacggcgttatactactgtgtcaccggactcgctgactactgggggcagggcactcttgtcacggtctccagc

SEQ ID NO: 23 - DNA, 735 bp, synthetic construct
  source 1..735: mol_type = other DNA
tgcgacattgttatgacgcagtcgccatcaagcttatcagcgtcagtgggagatcgcgttacaattacatgccgttcgagcactggggcagtcacaaccagtaattacgcttcgtgggtccaggaaaaaccgggtaagttgttcaagggtttgattggcggtactaataaccgcgcaccgggcgtccctagccgtttttcggggagtttgattggtgacaaggccacacttactatcagcagtctgcaaccagaggatttcgctacatacttttgtgcattgtggtactccaaccattgggtcttcggtcagggcacgaaggttgaacttaaacgcgggggtggtggctccggaggtgggggttcaggcggcggagggtcttcgggtggagggagtgaggttaagcttcttgaaagtggtggtggtcttgtgcagcctggaggctcgttaaagctgagctgcgctgtgagtggtttctcgttgacggattatggggtcaattgggtacgccaggcaccggggcgtggcttggagtggattggcgtcatctggggcgacggaatcactgattataacagtgccttgaaggatcgctttatcatcagcaaagacgattgcgaaaacactgtctatttgcaaatgagcaaagttcgctcggatgatacggcgttatactactgtgtcaccggattagctgactactgggggcagggcactcttgtcacggtctccagc

SEQ ID NO: 24 - DNA, 735 bp, synthetic construct
  source 1..735: mol_type = other DNA
tgcgacaatgttatgacgcagtcgccatcaagcttatcagcgtcagtgggagatcgcgttacaattacatgccgttcgagcactggggcagtcacaaccagtaattacgcttcgtgggtccaggaaaaaccgggtaaggtgttcaagggtttgattggcggtactaataaccgcgcaccgggcgtccctagccgtttttcggggagtttgattggtgacaaggccacacttactatcagcagtctgcaaccagaggatttcgctacatacttttgtgcattgtggtactccaaccattgggtcttcggtcagggcacgaaggttgaacttaaacgcgggggtggtggctccggaggtgggggttcaggcggcggagggtcttcgggtggagggagtgaggttaagcttcttgaaagtggtggtggtcttgtgcagcctggaggctcgttaaagctgagctgcgctgtgagtggtttctcgttgacggattatggggtcaattgggtacgccaggcaccggggcgtggcttggagtggattggcgtcatctggggcgacggaatcactgattataacagtgccttgaaggatcgctttatcatcagcaaagacgattgcgaaaacactgtctatttgcaaatgagcaaagttcgctcggatgatacggcgtcatactactgtgtcaccggattcgctgactactgggggcagggcactcttgtcacggtctccagc

SEQ ID NO: 25 - DNA, 735 bp, synthetic construct
  source 1..735: mol_type = other DNA
tgcgacattgttatgacgcagtcgccatcaagcttatcagcgtcagtgggagatcgcgttacaattacatgccgttcgagcactggggcagtcacaaccagtaattacgcttcgtgggtccaggaaaaaccgggtaagttgttcaagggtttgattggcggtactaataaccgcgcaccgggcgtccctagccgtttttcggggagtttgattggtgacaaggccacacttactatcagcagtctgcaaccagaggatttcgctacatacttttgtgcattgtggtactccaaccattgggtcttcggtcagggcacgaaggttgaacttaaacgcgggggtggtggctccggaggtgggggttcaggcggcggagggtcttcgggtggagggagtgaggttaagcttcttgaaagtggtggtggtcttgtgcagcctggaggctcgttaaagctgagctgcgctgtgagtggtttctcgttgacggattatggggtcaattgggtacgccaggcaccggggcgtggcttggagtggattggcgtcatctggggcgacggaatcactgattataacagtgccttgaaggatcgctttatcatcagcaaagacgattgcgaaaacactgtctatttgcaaatgagcaaagttcgctcggatgatacggcgtcatactactgtgtcaccggattagctgactactgggggcagggcactcttgtcacggtctccagc

SEQ ID NO: 26 - DNA, 744 bp, synthetic construct
  source 1..744: mol_type = other DNA
gacattcagatgacgcagtcgccatcaagcttaagcgccagtgtgggtgatcgcgtcacaatcacatgccgtgcttcccaagatgtaaataccgcggtggcctggtatcagcaaaaaccgggaaaagctccgaagcttttaatttacagtgcatcgttcctttatagcggggtcccaagccgcttttcgggttcgcgctccgggaccgacttcacgcttacgatttcaagcctgcaaccggaagatttcgccacatactattgccaacagcattacacgacgccgcctaccttcgggcaaggcacgaaggtggaaatcaaacgcgggggaggtggctccggaggtgggggttcaggcggcggagggtcttcgggtggagggagtgaggttcagcttgtggaatcaggtggaggtttagtgcaacctggtggtagtttacgcctgtcctgcgcagctagtggattcaatatcaaagacacttatatccattgggtacgtcaagcccctgggaaaggactggaatgggtcgcccgtatttaccccactaacggttatactcgttacgccgactctgttaagggacgcttcaccattagtgcggacacatctaaaaacacagcttacttgcagatgaactcccttcgtgcagaggacaccgccgtctactactgtagccgttggggaggggatggattttatgcgatggactactgggggcagggcactcttgtcacggtctccagc

SEQ ID NO: 27 - DNA, 810 bp, synthetic construct
  source 1..810: mol_type = other DNA
aaacaaagcactattgcactggcactcttaccgttactgtttatccctgtgactaaagccatgcgggacattcagatgacgcagtcgccatcaagcttaagcgccagtgtgggtgatcgcgtcacaatcacatgccgtgcttcccaagatgtaaataccgcggtggcctggtatcagcaaaaaccgggaaaagctccgaagcttttaatttacagtgcatcgttcctttatagcggggtcccaagccgtttttcgggttcgcgctccgggaccgacttcacgcttacgatttcaagcctgcaaccggaagatttcgccacatactattgccaacagtattacacgacgccgcctaccttcgggcaaggcacgaaggtggaaatcaaacgcgggggaggtggctccggaggtgggggttcaggcggcggagggtcttcgggtggagggagtgaggttcagcttgtggaatcaggtggaggtttagtgcaacctggtggtagtttacgcctgtcctgcgcagctagtggattcaatatcaaagacacttatatccattgggtacgtcaagcccctgggaaaggactggaatgggtcgcccgtatttaccccactaacggttatactcgttacgccgactctgttaagggacgcttcaccattagtgcggacacatctaaaaacacagcttacttgcagatgaactcccttcgtgcagaggacaccgccgtctactactgtagccgttggggaggggatggattttatgcgatggactactgggggcagggcactcttgtcacggtctccagc

SEQ ID NO: 28 - DNA, 1056 bp, synthetic construct
  source 1..1056: mol_type = other DNA
atgatcaaaattgccacgcgtaaatatttaggcaaacagaatgtttatgatatcggtgtcgagcgcgatcataatttcgcgctgaaaaacggctttatcgccagcaattgttttaatgcactcttaccgttactgtttacccctgtgactaaagacatgcgggacattcagatgacgcagtcgccatcaagcttaagcgccagtgtgggtgatcgcgtcacaatcacatgccgtgcttcccaagatgtaaataccgcggtggactggtatcagcaaaaaccgggaaaagctccgaagcttttaatttccagtgcatcgttcctttatagcggggtcccaagccgcttttcgggttcgcgctccgggaccgacttcacgcttacgatttcaagcctgcaaccggaagatttcgccacatactattgccaacagcattacacgacgccgcctaccttcgggcaaggcacgaaggtggaaatcaaacgcgggggaggtggctccggaggtgggggttcaggcggcggagggtcttcgggtggagggagtgaggttcagcttgtggaatcaggtggaggtttagtgcaacctggtggtagtttacgcctgtcctgcgcagctagtggattcaatatcaaagacacttatatccattgggtacgtcaagcccctgggaaaggactggaatgggtcgcccgtatttaccccactaacggttatactcgttacgccgactctgttaagggacgcttcaccattagtgcggacacatctaaaaacacagcttacttgcagatgaactcccttcgtgcagaggacaccgccgtctactactgtagccgttggggaggggatggattttatgcgatggactactgggggcagggcactcttgtcacggtctccagcggtggatcaggcggaagtggcggttcaggtgggagtggtggcagcttgcaaagaatgaaacaacttgaagacaaggttgaagaattgctttcgaaaaattatcacttggaaaatgaggttgccagattaaagaaattagttggcgaacgc

SEQ ID NO: 29 - DNA, 744 bp, synthetic construct
  source 1..744: mol_type = other DNA
gacattcagatgacgcagtcgccatcaagcttaagcgccagtgtgggtgatcgcgtcacaatcacatcccgtgcttcccaagatgtaaataccgcggtggcctggtatcagcaaaaaccgggaaaagctccgaagcttttaatttacagtgcatcgttcctttatagcggggtcccaagccgcttttcgggttcgcgctccgggaccgacttcacgcttacgatttcaagcctgcaaccggaagatttcgccacatactattcccaacagcattacacgacgccgcctaccttcgggcaaggcacgaaggtggaaatcaaacgcgggggaggtggctccggaggtgggggttcaggcggcggagggtcttcgggtggagggagtgaggttcagcttgtggaatcaggtggaggtttagtgcaacctggtggtagtttacgcctgtcctcagcagctagtggattcaatatcaaagacacttatatccattgggtacgtcaagcccctgggaaaggactggaatgggtcgcccgtatttaccccactaacggttatactcgttacgccgactctgttaagggacgcttcaccattagtgcggacacatctaaaaacacagcttacttgcagatgaactcccttcgtgcagaggacaccgccgtctactactctagccgttggggaggggatggattttatgcgatggactactgggggcagggcactcttgtcacggtctccagc

SEQ ID NO: 30 - DNA, 36 bp, synthetic construct
  source 1..36: mol_type = other DNA
cttctggggccatacgaattatgggaattaagtcac

SEQ ID NO: 31 - DNA, 45 bp, synthetic construct
  source 1..45: mol_type = other DNA
ggtggatcaggcggaagtggcggttcaggtgggagtggtggcagc

SEQ ID NO: 32 - protein, 20 aa, synthetic construct
KQSTIALALLPLLFTPVTKA

SEQ ID NO: 33 - protein, 512 aa, synthetic construct
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRFTTFWVWFFFLLSLGICVALVAFSSLDTRLPMSKSRILLNPRDIDINMVNKSCNSWSSPYQLSYAIGVGDLVATSLNTFSTFMVHDKINYNIDEPSSSGKTLSIAFVNQRQYRAQQCFMSIKLVDNADGSTMLDKRYVITNGNQLAIQNDLLESLSKALNQPWPQRMQETLQKILPHRGALLTNFYQAHDYLLHGDDKSLNRASELLGEIVQSSPEFTYARAEKALVDIVRHSQHPLDEKQLAALNTEIDNIVTLPELNNLSIIYQIKAVSALVKGKTDESYQAINTGIDLEMSWLNYVLLGKVYEMKGMNREAADAYLTAFNLRPGANTLYWIENGIFQTSVPYVVPYLDKFLASE

SEQ ID NO: 34 - DNA, 24 bp, synthetic construct
  source 1..24: mol_type = other DNA
cgaaccagaaaagaaccattaatg

SEQ ID NO: 35 - protein, 333 aa, synthetic construct
SLDTRLPMSKSRILLNPRDIDINMVNKSCNSWSSPYQLSYAIGVGDLVATSLNTFSTFMVHDKINYNIDEPSSSGKTLSIAFVNQRQYRAQQCFMSIKLVDNADGSTMLDKRYVITNGNQLAIQNDLLESLSKALNQPWPQRMQETLQKILPHRGALLTNFYQAHDYLLHGDDKSLNRASELLGEIVQSSPEFTYARAEKALVDIVRHSQHPLDEKQLAALNTEIDNIVTLPELNNLSIIYQIKAVSALVKGKTDESYQAINTGIDLEMSWLNYVLLGKVYEMKGMNREAADAYLTAFNLRPGANTLYWIENGIFQTSVPYVVPYLDKFLASE

SEQ ID NO: 36 - DNA, 39 bp, synthetic construct
  source 1..39: mol_type = other DNA
aaacattaaatgtttatcttttcatgatatcaacttgcg

SEQ ID NO: 37 - DNA, 30 bp, synthetic construct
  source 1..30: mol_type = other DNA
cctcaagttctcacttacagaaacttttgt

SEQ ID NO: 38 - DNA, 25 bp, synthetic construct
  source 1..25: mol_type = other DNA
  modified_base 19: mod_base = OTHER; note = Uracil
atgttgaaaatctccttctagatta

SEQ ID NO: 39 - DNA, 16 bp, synthetic construct
  source 1..16: mol_type = other DNA
  modified_base 11: mod_base = OTHER; note = Uracil
aggtgggggttcaggc

SEQ ID NO: 40 - DNA, 17 bp, synthetic construct
  source 1..17: mol_type = other DNA
  modified_base 11: mod_base = OTHER; note = Uracil
aacccccacctccggag

SEQ ID NO: 41 - DNA, 19 bp, synthetic construct
  source 1..19: mol_type = other DNA
gactccctgcaagcctcag

SEQ ID NO: 42 - protein, 179 aa, synthetic construct
MQQPVVRVGEWLVTPSINQISRNGRQLTLEPRLIDLLVFFAQHSGEVLSRDELIDNVWKRSIVTNHVVTQSISELRKSLKDNDEDSPVYIATVPKRGYKLMVPVIWYSEEEGEEIMLSSPPPIPEAVPATDSPSHSLNIQNTATPPEQSPVKSKRGGPGLLLLLLLLLLLLLLLLGPGG

SEQ ID NO: 43 - protein, 4 aa, synthetic construct
GGGS